(19) United States
(12) Patent Application Publication
(10) Pub. No.: US 2023/0121103 A1
(43) Pub. Date: Apr. 20, 2023

(54) SYSTEMS AND METHODS FOR CANCER WHOLE GENOME AND TRANSCRIPTOME SEQUENCING (CWGTS)

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventors: Max Levine, New York, NY (US); Gunes Gundem, New York, NY (US); Yangyu Zhou, Boston, MA (US); Juan Esteban Arango Ossa, New York, NY (US); Juan Santiago Medina-Martinez, New York, NY (US); Jesus Gutierrez-Abril, New York, NY (US); Elli Papaemmanuil, New York, NY (US); Andrew Kung, New York, NY (US); Neerav Shukla, New York, NY (US)

(21) Appl. No.: 17/857,821

(22) Filed: Jul. 5, 2022

Related U.S. Application Data
(60) Provisional application No. 63/257,910, filed on Oct. 20, 2021.

(51) Int. Cl. G16H 20/40; G16B 20/10; G16B 20/20; G16B 25/10; G16H 15/00

(52) U.S. Cl. CPC G16H 20/40; G16B 20/10; G16B 20/20; G16B 25/10; G16H 15/00

(57) ABSTRACT

Described embodiments provide systems and methods for performing cancer whole genome and transcriptome sequencing (cWGTS). A plurality of datasets can be generated based on sequencing of a tumor sample and a healthy control germline sample. A plurality of databases, comprising a first, second and third database, can be accessed. An RNA gene expression analysis can be performed to generate a first plurality of outputs. A DNA ploidy and allelic imbalance analysis can be performed to generate a second plurality of outputs. A variant calling analysis can be performed to generate a third plurality of outputs. A workflow may be implemented. Cohort classification scores and disease-specific classification scores for each individual level output in each of the first, second, and third pluralities of outputs can be generated. A report can be generated and provided to one or more users.

### CROSS REFERENCE TO RELATED APPLICATION

The present application claims the benefit of priority to U.S. Pat. Provisional Application No. 63/257,910, titled “SYSTEMS AND METHODS FOR CANCER WHOLE GENOME AND TRANSCRIPTOME SEQUENCING (CWGTS),” filed Oct. 20, 2021, which is incorporated herein in its entirety by reference.

### TECHNICAL FIELD

The present technology relates generally to whole genome sequencing (WGS) and whole transcriptome sequencing for determination of anti-cancer therapy.

### BACKGROUND

The following description of the background of the present technology is provided simply as an aid in understanding the present technology and is not admitted to describe or constitute prior art to the present technology.

Cancer is caused by the accumulation of somatic variants including point mutations, small insertion/deletions, structural variants (SVs) and copy number alterations (CNAs) that drive oncogenesis, disease progression, and in some cases define therapeutic vulnerabilities. The introduction of next-generation sequencing (NGS)-based targeted gene panel assays has aided disease diagnosis, guided care, and improved patient outcomes through refinement of treatment options. However, targeted panels are optimized to assess clinical biomarkers in common cancers. In contrast, for patients with pediatric or rare cancers that have low mutation burden and are primarily driven by structural variants and fusion genes, panel tests fail to identify a clinical biomarker in most cases. This underscores an unmet need for enhanced workflows to guide clinical management.

### SUMMARY

This Summary is provided to introduce a selection of concepts in a simplified form that are further described below in the Detailed Description. This Summary is not intended to identify key features or essential features, nor is it intended to limit the scope of the claims included herewith.

The present disclosure is directed towards systems and methods for determining anti-cancer therapy and/or treatment, for example, using cancer whole genome and transcriptome sequencing (cWGTS). Cancer whole genome and transcriptome sequencing can be used and/or applied in certain medical areas, such as oncology. However, several challenges can be considered when implementing and/or developing cWGTS technology, such as the need to deliver results within clinically relevant timeframes, concerns about assay sensitivity, interpretation, reporting and/or prioritization of cWGTS findings. The systems and methods described herein include an approach for reporting, providing, and/or indicating comprehensive cWGTS results in a matter of days (e.g., 9 or fewer days, or 3 or fewer days for the analysis), thereby delivering results within clinically relevant timeframes. Embodiments of said systems and methods can demonstrate a comparable sensitivity to panel assays for the detection of clinically relevant mutations. Benchmarking can identify an optimal depth of at least 80x for clinical whole genome sequencing (WGS) sequencing. Integration of germline, somatic DNA and RNA-seq data into said approach can enable data-driven variant prioritization and reporting, with oncogenic findings reported in 54% (or other percentage values) more patients than standard of care. In certain embodiments, cell-free DNA (cfDNA) can be used as an alternative source of tumor DNA for WGS. The systems and methods presented herein provide key technical considerations and contributions for implementing and/or including cWGTS technology as an integrated test in clinical oncology.

In one aspect, the present disclosure is directed to a computer-implemented method for determining anti-cancer therapy and/or treatment using cancer whole genome and transcriptome sequencing (cWGTS). The method may include (A) generating, based on sequencing of a tumor sample and a healthy control germline sample, a plurality of datasets. The plurality of datasets may comprise (1) a first dataset based on whole transcriptome sequencing of RNA in the tumor sample obtained from a patient, (2) a second dataset based on a whole genome sequencing (WGS) of DNA derived from the tumor sample obtained from the patient, and (3) a third dataset based on WGS of DNA in the healthy control germline sample. The method may include (B) accessing a plurality of databases. The plurality of databases may include (1) a first reference database comprising, for a reference cohort of tumor samples, a plurality of individual sample gene expression transcripts per million (TPM) values, (2) a second reference database comprising, for a reference cohort of tumor samples, at an individual sample level, annotations for at least one of (i) RNA fusions, (ii) somatic structural variants, (iii) somatic substitutions, (iv) somatic insertions and deletions (indels), (v) microsatellite instability and/or mutational burden scores for each variant class, (vi) germline variants, (vii) somatic mutation patterns or signatures in each sample, or (vii) allelic imbalances, and (3) a third database comprising a plurality of gene identifiers corresponding to a plurality of known cancer genes. The method may include (C) performing an RNA gene expression analysis using the first dataset, the first reference database, and the third database, to generate, for the tumor sample, a first plurality of outputs. The first plurality of outputs can be generated based on: (1) detection of established cancer genes having aberrant gene expression in the tumor sample relative to that observed in normal control subjects, and (2) prioritization of the detected aberrantly-expressed cancer genes in the tumor sample.

The method may include (D) performing a DNA ploidy and allelic imbalance analysis using the second dataset, the third dataset, and the third database, to generate, for the tumor sample, a second plurality of outputs. The second plurality of outputs can be generated based on (1) detection of high-confidence aberrant copy number segments in the tumor sample by applying one or more allelic imbalance identification techniques, and (2) prioritization of allelic imbalances in the tumor sample based on a set of criteria comprising an overlap of the high-confidence aberrant copy number segments in the tumor sample with the known cancer genes in the third database. The method may include (E) performing, based on the RNA gene expression analysis of Step (C) and the DNA ploidy and allelic imbalance analysis of Step (D), a variant calling analysis to generate, for the tumor sample, a third plurality of outputs. The third plurality of outputs may be generated based on: (1) detection of RNA fusions, (2) detection of somatic structural variants, (3) detection of somatic substitutions, (4) detection of somatic insertions and deletions (indels), (5) assessment of microsatellite instability and/or mutational burden across variant classes, (6) detection of germline variants, (7) clonality analysis, (8) determination of a number of structural variants and gene fusions in the DNA of the tumor sample, and/or (9) determination of somatic mutation patterns or signatures in the tumor sample. The method may include (F) implementing a workflow. The workflow may comprise (1) identifying orthogonal supportive indicators based on consistency of two or more outputs in at least two of the first, second, and third pluralities of outputs generated in Step (C), Step (D), and Step (E), respectively, (2) prioritizing genetic alterations based on the orthogonal supportive indicators, (3) generating global classifications based on the orthogonal supportive indicators, and (4) classifying at least one somatic mutation in an established cancer gene that is detected in both the first dataset and the second dataset as being orthogonally validated.

The method may include (G) generating cohort classification scores for each individual level output in each of the first, second, and third pluralities of outputs for the tumor sample based on the reference cohort of tumor samples in at least one of the first reference database or the second reference database. The method may include (H) generating disease-specific classification scores for each individual level output in each of the first, second, and third pluralities of outputs for the tumor sample based on a subset of the reference cohort of tumor samples in at least one of the first reference database or the second reference database, wherein the subset of the reference cohort of tumor samples is of a same cancer type. The method may include (I) generating a report comprising, for the tumor sample, the prioritized allelic imbalances (see, e.g., FIG. 50), the microsatellite instability and/or mutational burden across variant classes (see, e.g., FIGS. 22A and 46A), the germline variants (see, e.g., FIGS. 31 and 43B), outputs of the clonality analysis (see, e.g., FIG. 48C), the number of structural variants and gene fusions in the DNA (see, e.g., FIGS. 22A and 32), the somatic mutation patterns or signatures (see, e.g., FIGS. 43A and 46B), the at least one orthogonally-validated somatic mutation (see, e.g., FIG. 18), the cohort classification scores (see, e.g., FIG. 50), and the disease specific classification scores (see, e.g., FIG. 41). The method may include (J) providing the report to one or more users for determination of an anti-cancer therapy, wherein providing the report comprises at least one of (1) transmitting the report to a computing device, (2) displaying the report on a display screen, or (3) storing the report in a non-volatile computer-readable storage medium.

In certain embodiments, generating the cohort classification scores may further comprise: (1) interrogating the at least one orthogonally-validated somatic mutation against a fourth database that associates a plurality of somatic mutations with a plurality of specific cancer types or pan-cancer markers; and (2) identifying the at least one orthogonally-validated somatic mutation as associated with a specific cancer type or pan-cancer hotspot when there is a match. In some embodiments, generating disease-specific classification scores may further comprise performing a t-distributed stochastic neighbor embedding (tSNE) analysis. In certain embodiments, the method may further comprise classifying germline mutation pathogenicity by integration of data derived in Step 1(E) relating to acquired somatic mutation patterns or signatures. In some embodiments, the method may further comprise determining the anti-cancer therapy based on values used for the report, and providing the anti-cancer therapy in the report (e.g., Poly (ADP-ribose) polymerase (PARP) inhibitors (PARPi) for a germline BReast CAncer gene (BRCA), immunotherapy for high TMB, etc.). In certain embodiments, the anti-cancer therapy can be determined based on interrogation of an external therapy database to identify a therapy that aligns with the outputs in the report. In some embodiments, the tumor sample may be a clinical sample selected from the group consisting of fresh tissue, frozen tissue, formalin-fixed paraffin-embedded tissue, blood, cfDNA, plasma, and serum. In certain embodiments, the method may further comprise determining that the second and third datasets have at least one of (1) a quality score satisfying a quality threshold, (2) a coverage metric satisfying a coverage threshold, or (3) a tumor cell content satisfying a tumor purity threshold. In some embodiments, the quality score may indicate genome mapping quality, and the quality threshold is at least 20 Phred. In certain embodiments, the coverage score can indicate genome coverage, and the coverage threshold is at least about 70% genome coverage. In some embodiments, the tumor cell content may indicate tumor purity corresponding to the DNA in the tumor sample, and the tumor purity threshold is at least about 20% tumor purity.

In certain embodiments, the method may further comprise determining that the second and third datasets have: (1) a quality score satisfying a quality threshold; (2) a coverage metric satisfying a coverage threshold; and (3) a tumor cell content satisfying a tumor purity threshold. In some embodiments, performing the RNA gene expression analysis may further comprise detecting over-expressed or under-expressed genes based on the TPM values satisfying a percentile threshold relative to the first reference database. In certain embodiments, the set of criteria for prioritizing allelic imbalances in the tumor sample may further include whole-genome duplication (WGD). In some embodiments, the set of criteria for prioritizing allelic imbalances in the tumor sample may further include an aberrant copy number segment having a direction that is consistent with cancer gene function. In certain embodiments, the variant calling analysis may comprise detection of RNA fusions, wherein detection of RNA fusions comprises employing a plurality of independent fusion gene callers on raw data. In some embodiments, the variant calling analysis may comprise detection of RNA fusions, wherein detection of RNA fusions comprises detection of high-confidence fusion genes, and employing a rescue process to recover detected high-confidence fusion genes that were not detected by at least two independent variant callers as a reference for known cancer genes, wherein rescued fusions are required to have at least one spanning read. In some embodiments, the variant calling analysis may comprise detection of somatic structural variants, wherein detection of somatic structural variants comprises deploying a plurality of independent structural variant callers on raw data.

In certain embodiments, the variant calling analysis may comprise detection of somatic structural variants, wherein detection of somatic structural variants comprises selection of high-confidence structural variants by merging all calls having more than a first predetermined number of base pairs (bp) by a window that includes a breakpoint, the window having a size that is a second predetermined number of bps. In some embodiments, the variant calling analysis may comprise detection of somatic substitutions, wherein detection of somatic substitutions comprises employing a plurality of independent substitution callers on raw data. In certain embodiments, the variant calling analysis may comprise detection of somatic indels, wherein detection of somatic indels comprises generating one or more indel signatures and using the one or more indel signatures to determine if a somatic indel is a repeat-mediated deletion, a microhomology association, or an insertion. In some embodiments, the variant calling analysis may comprise assessment of microsatellite instability and/or mutational burden across variant classes. In certain embodiments, the method may further comprise determining structural variant (SV) burden by collapsing complex structural variants into unique structural variant clusters to avoid over estimation of structural variant burden. In some embodiments, the variant calling analysis may comprise detection of germline variants, wherein detection of germline variants comprises deploying a plurality of independent germline callers on raw data. In some embodiments, the clonality analysis may comprise using purity and local copy numbers to scale variant allele frequency (VAF) of single nucleotide variants (SNVs) and indels to cancer cell fraction (CCF) for one or more tumor samples from the patient. In certain embodiments, (1) candidate driver mutations, (2) aberrant copy number segments, and (3) structural variants can be assigned to each clone to generate clone-specific mutation profiles.

In another aspect, the present disclosure is directed to a computing system for determining anti-cancer therapy and/or treatment using cancer whole genome and transcriptome sequencing (cWGTS). The computing system may comprise one or more processors and a computer-readable memory having instructions stored thereon. The one or more processors can execute the instructions stored on the computer-readable memory. Upon execution of the instructions by the one or more processors, the instructions may cause the computing system to (A) generate, based on sequencing of a tumor sample and a healthy control germline sample, a plurality of datasets. The plurality of datasets may comprise (1) a first dataset based on whole transcriptome sequencing of RNA in the tumor sample obtained from a patient, (2) a second dataset based on a whole genome sequencing (WGS) of DNA derived from the tumor sample obtained from the patient, and (3) a third dataset based on WGS of DNA in the healthy control germline sample. The instructions may cause the one or more processors to (B) access a plurality of databases. The plurality of databases may comprise (1) a first reference database comprising, for a reference cohort of tumor samples, a plurality of individual sample gene expression transcripts per million (TPM) values, (2) a second reference database comprising, for a reference cohort of tumor samples, at an individual sample level, annotations for at least one of (i) RNA fusions, (ii) somatic structural variants, (iii) somatic substitutions, (iv) somatic insertions and deletions (indels), (v) microsatellite instability and/or mutational burden scores for each variant class, (vi) germline variants, (vii) somatic mutation patterns or signatures in each sample, or (vii) allelic imbalances, and (3) a third database comprising a plurality of gene identifiers corresponding to a plurality of known cancer genes. The instructions may cause the one or more processors to (C) perform an RNA gene expression analysis using the first dataset, the first reference database, and the third database, to generate, for the tumor sample, a first plurality of outputs. The first plurality of outputs can be generated based on: (1) detection of established cancer genes having aberrant gene expression in the tumor sample relative to that observed in normal control subjects, and (2) prioritization of the detected aberrantly-expressed cancer genes in the tumor sample.

The instructions may cause the one or more processors to (D) perform a DNA ploidy and allelic imbalance analysis using the second dataset, the third dataset, and the third database, to generate, for the tumor sample, a second plurality of outputs. The second plurality of outputs can be generated based on (1) detection of high-confidence aberrant copy number segments in the tumor sample by applying one or more allelic imbalance identification techniques, and (2) prioritization of allelic imbalances in the tumor sample based on a set of criteria comprising an overlap of the high-confidence aberrant copy number segments in the tumor sample with the known cancer genes in the third database. The instructions may cause the one or more processors to (E) perform, based on the RNA gene expression analysis of Step (C) and the DNA ploidy and allelic imbalance analysis of Step (D), a variant calling analysis to generate, for the tumor sample, a third plurality of outputs. The third plurality of outputs can be generated based on (1) detection of RNA fusions, (2) detection of somatic structural variants, (3) detection of somatic substitutions, (4) detection of somatic insertions and deletions (indels), (5) assessment of microsatellite instability and/or mutational burden across variant classes, (6) detection of germline variants, (7) clonality analysis, (8) determination of a number of structural variants and gene fusions in the DNA of the tumor sample, and/or (9) determination of somatic mutation patterns or signatures in the tumor sample.

The instructions may cause the one or more processors to (F) implement a workflow. The workflow may comprise (1) identifying orthogonal supportive indicators based on consistency of two or more outputs in at least two of the first, second, and third pluralities of outputs generated in Step (C), Step (D), and Step (E), respectively, (2) prioritizing genetic alterations based on the orthogonal supportive indicators, (3) generating global classifications based on the orthogonal supportive indicators, and (4) classifying at least one somatic mutation in an established cancer gene that is detected in both the first dataset and the second dataset as being orthogonally validated. The instructions may cause the one or more processors to (G) generate cohort classification scores for each individual level output in each of the first, second, and third pluralities of outputs for the tumor sample based on the reference cohort of tumor samples in at least one of the first reference database or the second reference database. The instructions may cause the one or more processors to (H) generate disease-specific classification scores for each individual level output in each of the first, second, and third pluralities of outputs for the tumor sample based on a subset of the reference cohort of tumor samples in at least one of the first reference database or the second reference database, wherein the subset of the reference cohort of tumor samples is of a same cancer type. The instructions may cause the one or more processors to (I) generate a report comprising, for the tumor sample, the prioritized allelic imbalances, the microsatellite instability and/or mutational burden across variant classes, the germline variants, outputs of the clonality analysis, the number of structural variants and gene fusions in the DNA, the somatic mutation patterns or signatures, the at least one orthogonally-validated somatic mutation, the cohort classification scores, and the disease specific classification scores. The instructions may cause the one or more processors to (J) provide the report to one or more users for determination of an anti-cancer therapy for the patient by at least one of (1) transmitting the report to a computing device, (2) displaying the report on a display screen, or (3) storing the report in a non-volatile computer-readable storage medium.

In certain embodiments, the instructions can be further configured to cause the one or more processors to generate the cohort classification scores by: (1) interrogating the at least one orthogonally-validated somatic mutation against a fourth database that associates a plurality of somatic mutations with a plurality of specific cancer types or pan-cancer markers, and (2) identifying the at least one orthogonally-validated somatic mutation as associated with a specific cancer type or pan-cancer hotspot when there is a match. In some embodiments, the instructions can be further configured to cause the one or more processors to generate disease-specific classification scores by performing a t-distributed stochastic neighbor embedding (tSNE) analysis. In certain embodiments, the instructions can be further configured to cause the one or more processors to classify germline mutation pathogenicity by integration of data derived in Step 1(E) relating to acquired somatic mutation patterns or signatures. In some embodiments, the instructions can be further configured to cause the one or more processors to determine the anti-cancer therapy based on values used for the report, and provide the anti-cancer therapy in the report. In certain embodiments, the instructions can be further configured to cause the one or more processors to determine the anti-cancer therapy by interrogating an external therapy database to identify a therapy that aligns with the outputs in the report. In some embodiments, the tumor sample may be a clinical sample selected from the group consisting of fresh tissue, frozen tissue, formalin-fixed paraffin-embedded tissue, blood, cfDNA, plasma, and serum.

In certain embodiments, the instructions can be further configured to cause the one or more processors to determine that the second and third datasets have at least one of (1) a quality score satisfying a quality threshold, (2) a coverage metric satisfying a coverage threshold, or (3) a tumor cell content satisfying a tumor purity threshold. In some embodiments, the quality score may indicate genome mapping quality, and the quality threshold is at least 20 Phred. In certain embodiments, the coverage score may indicate genome coverage, and the coverage threshold is at least about 70% genome coverage. In some embodiments, the tumor cell content may indicate tumor purity corresponding to the DNA in the tumor sample, and the tumor purity threshold is at least about 20% tumor purity. In certain embodiments, the instructions can be further configured to cause the one or more processors to determine that the second and third datasets have: (1) a quality score satisfying a quality threshold; (2) a coverage metric satisfying a coverage threshold; and (3) a tumor cell content satisfying a tumor purity threshold. In some embodiments, the instructions can be further configured to cause the one or more processors to perform the RNA gene expression analysis by detecting over-expressed or under-expressed genes based on the TPM values satisfying a percentile threshold relative to the first reference database. In certain embodiments, the set of criteria for prioritizing allelic imbalances in the tumor sample may further include whole-genome duplication (WGD).

In some embodiments, the set of criteria for prioritizing allelic imbalances in the tumor sample may further include an aberrant copy number segment having a direction that is consistent with cancer gene function. In certain embodiments, the instructions can be further configured to cause the one or more processors to perform the variant calling analysis by detecting RNA fusions, wherein detecting RNA fusions comprises employing a plurality of independent fusion gene callers on raw data. In some embodiments, the instructions can be further configured to cause the one or more processors to perform the variant calling analysis by detecting RNA fusions, wherein detecting RNA fusions comprises detecting high-confidence fusion genes, and employing a rescue process to recover detected high-confidence fusion genes that were not detected by at least two independent variant callers as a reference for known cancer genes, wherein rescued fusions are required to have at least one spanning read. In certain embodiments, the instructions can be further configured to cause the one or more processors to perform the variant calling analysis by detecting somatic structural variants, wherein detecting somatic structural variants comprises deploying a plurality of independent structural variant callers on raw data. In some embodiments, the instructions can be further configured to cause the one or more processors to perform the variant calling analysis by detecting somatic structural variants, wherein detecting somatic structural variants comprises selecting high-confidence structural variants by merging all calls having more than a first predetermined number of base pairs (bp) by a window that includes a breakpoint, the window having a size that is a second predetermined number of bps. In certain embodiments, the instructions can be further configured to cause the one or more processors to perform the variant calling analysis by detecting somatic substitutions, wherein detecting somatic substitutions comprises employing a plurality of independent substitution callers on raw data.

In some embodiments, the instructions can be further configured to cause the one or more processors to perform the variant calling analysis by detecting somatic indels, wherein detecting somatic indels comprises generating one or more indel signatures and using the one or more indel signatures to determine if a somatic indel is a repeat-mediated deletion, a microhomology association, or an insertion. In certain embodiments, the instructions can be further configured to cause the one or more processors to perform the variant calling analysis by assessing microsatellite instability and/or mutational burden across variant classes. In some embodiments, the instructions can be further configured to cause the one or more processors to determine structural variant (SV) burden by collapsing complex structural variants into unique structural variant clusters to avoid over estimation of structural variant burden. In certain embodiments, the instructions can be further configured to cause the one or more processors to perform the variant calling analysis by detecting germline variants, wherein detecting germline variants comprises deploying a plurality of independent germline callers on raw data. In some embodiments, the instructions can be further configured to cause the one or more processors to perform the clonality analysis by using purity and local copy numbers to scale variant allele frequency (VAF) of single nucleotide variants (SNVs) and indels to cancer cell fraction (CCF) for one or more tumor samples from the patient. In certain embodiments, the instructions can be further configured to cause the one or more processors to generate clone-specific mutation profiles by assigning (1) candidate driver mutations, (2) aberrant copy number segments, and (3) structural variants to each clone.

In another aspect, the present disclosure is directed to a computer-implemented method for determining anti-cancer therapy and/or treatment using cancer whole genome and transcriptome sequencing (cWGTS). The computer-implemented method may include (A) generating, by one or more processors of a computing system, based on sequencing of a tumor sample and a healthy control germline sample, a plurality of datasets. The plurality of datasets may comprise (1) a first dataset based on whole transcriptome sequencing of RNA in the tumor sample obtained from a patient, (2) a second dataset based on a whole genome sequencing (WGS) of DNA derived from the tumor sample obtained from the patient, and (3) a third dataset based on WGS of DNA in the healthy control germline sample. The computer-implemented method may include (B) performing, by the one or more processors, an RNA gene expression analysis using the first dataset to generate, for the tumor sample, a first plurality of outputs. The first plurality of outputs can be generated based on detection of established cancer genes having aberrant gene expression in the tumor sample relative to that observed in normal control subjects. The computer-implemented method may include (C) performing, by the one or more processors, a DNA ploidy and allelic imbalance analysis using the second dataset to generate, for the tumor sample, a second plurality of outputs. The second plurality of outputs can be generated based on detection of high-confidence aberrant copy number segments in the tumor sample by applying one or more allelic imbalance identification techniques.

The computer-implemented method may include (D) performing, by the one or more processors, based on the RNA gene expression analysis of Step (B) and the DNA ploidy and allelic imbalance analysis of Step (C), a variant calling analysis to generate, for the tumor sample, a third plurality of outputs. The third plurality of outputs can be generated based on a plurality of: (1) detection of RNA fusions; (2) detection of somatic structural variants; (3) detection of somatic substitutions; (4) detection of somatic insertions and deletions (indels); (5) assessment of microsatellite instability and/or mutational burden across variant classes; (6) detection of germline variants; (7) clonality analysis; (8) determination of a number of structural variants and gene fusions in the DNA of the tumor sample; and/or (9) determination of somatic mutation patterns or signatures in the tumor sample. The computer-implemented method may include (E) implementing, by the one or more processors, a workflow. The workflow may comprise (1) identifying, by the one or more processors, orthogonal supportive indicators based on consistency of two or more outputs in at least two of the first, second, and third pluralities of outputs generated in Steps (B), (C), and (D), respectively, and (2) classifying, by the one or more processors, at least one somatic mutation in an established cancer gene that is detected in both the first dataset and the second dataset as being orthogonally validated. The computer-implemented method may include (F) generating, by the one or more processors, cohort classification scores for each individual level output in each of the first, second, and third pluralities of outputs for the tumor sample based on a reference cohort of tumor samples.

The computer-implemented method may include (G) generating, by the one or more processors, disease-specific classification scores for each individual level output in each of the first, second, and third pluralities of outputs for the tumor sample based on a subset of the reference cohort of tumor samples, wherein the subset of the reference cohort of tumor samples is of a same cancer type. The computer-implemented method may include (H) generating, by the one or more processors, a report, for the tumor sample, based on Steps (A)-(G). The report may comprise information corresponding to a plurality of allelic imbalances, microsatellite instability and/or mutational burden across variant classes, germline variants, clonality analysis, structural variants and gene fusions in the DNA, somatic mutation patterns or signatures, orthogonally-validated somatic mutations, cohort classification scores, and disease specific classification scores. The computer-implemented method may include (I) providing, by the one or more processors, the report to one or more users for determination of an anti-cancer therapy, wherein providing the report comprises at least one of (1) transmitting, by the one or more processors, the report to a computing device, (2) displaying, by the one or more processors, the report on a display screen, or (3) storing the report in a non-volatile computer-readable storage medium of the computing system.

The computer-implemented method may further comprise determining the anti-cancer therapy based on values used for the report, and providing the anti-cancer therapy in the report. In certain embodiments, the anti-cancer therapy may be determined based on interrogation of an internal or external therapy database to identify a therapy that aligns with the outputs in the report.

In various embodiments, the present disclosure is directed to a computing system (comprising one or more computing devices) comprising one or more processors, the computing system configured to implement any of the above methods. In various embodiments, the computing system may comprise a computer-readable memory comprising instructions configured to cause the one or more processors to implement any of the above methods. In various embodiments, the present disclosure is directed to a non-transitory computer-readable storage medium comprising instructions configured to cause one or more processors of a computing system (comprising one or more computing devices) to implement any of the above methods.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1 depicts a block diagram of a server system and a client computer system according to an illustrative embodiment.

FIG. 2 depicts a diagram of an example process as described in Step I, according to potential embodiments.

FIG. 3 depicts a diagram of an example process as described in Step II, in which a workflow summarizing a process for deriving gene expression-based metrics is discussed, according to potential embodiments.

FIG. 4 depicts a diagram of an example process as described in Step III, according to potential embodiments.

FIG. 5 depicts a diagram of an example process as described in Step IV, according to potential embodiments.

FIG. 6 depicts a diagram of an example process as described in Step IV.A, according to potential embodiments.

FIG. 7 depicts a diagram of an example process as described in Step IV.B, according to potential embodiments.

FIG. 8 depicts a plurality of high-confidence structural variants affecting the TP53 locus, according to potential embodiments.

FIG. 9 depicts a diagram of an example process as described in Step IV.C, according to potential embodiments.

FIG. 10 depicts a diagram of an example process as described in Step IV.D, according to potential embodiments.

FIG. 11 depicts a diagram of an example process as described in Step VI, according to potential embodiments.

FIG. 12 depicts a diagram of an example process as described in Step VII, according to potential embodiments.

FIG. 13 depicts a diagram of an example process as described in Step IX, according to potential embodiments.

FIGS. 14-22B depict the genomic findings of the subjects of Case Studies 1 to 12, according to potential embodiments.

FIG. 23 depicts characteristics of at least a portion of a study cohort, according to potential embodiments.

FIGS. 24A-24C depict an overview of the study cohort, according to potential embodiments, a) Bar chart displaying distribution of patients across main oncotree disease code and color-coded by gender. b) Bar chart showing the breakdown of treatment status by disease stage (Diagnosis, Primary refractory and Relapse). c) Tile plot summarizing molecular profiling assays run on each of the patient samples. Clinical MSK-IMPACT is used when available, otherwise Research MSK-IMPACT was used. d) Swarm style plot showing median coverage assessed by Mosdepth for the WGS sequencing data derived from tumor and normal samples in study.

FIGS. 25A-25B depict an end-to-end overview of a cWGTS workflow, according to potential embodiments, a) Schematic representation of the end-to-end cWGTS workflow, with information on median time duration (in hours) for each step, as determined by a time trial over four consecutive batches and representation of dedicated resources necessary to execute the workflow. b) Comparison of best reported turnaround times, from sample collection to results ready for tumor board review, of our cWGTS workflow relative to literature. Orange bar shows median time for the 16 samples with minimum and maximum times denoted with the error bar.

FIG. 26 depicts an overview of a cWGTS biomarker, according to potential embodiments, a) Heatmap representing the presence of genomic markers by cWGTS, with columns as samples and rows as markers, to include: 1. Somatic copy number aberrations (CNA). 2. Somatic SNVs/indels affecting cancer genes (Cancer Gene). 3. Somatic structural variants targeting cancer genes (SV). 4. Somatic oncogenic gene fusions (Fusion). 5. Clinically relevant SNVs and indels in established germline predisposition genes (Germline). 6. Coding tumor mutation burden (TMB). 7. Microsatellite instability (MSI). 8. Informative signatures (e.g., mutation signatures associated with mutations in DNA repair genes) (Inform. Sig.). 9. Whole genome duplication (WGD). 10. Chromothripsis (Chromo.). 11. Aberrant telomere length between germline and tumor samples (Telomere). 12. Evidence of viral integration sequences in RNA-seq (Viral). 13. Treatment-related signatures (e.g., mutation signatures associated with platinum or temozolomide exposure in tumor) (Treat. Sig.) Bar plot on the right shows the proportion of patients whose tumor harbors each genomic marker. The single sample in our cohort for which an oncogenic driver was not found, was an Epstein-Barr virus (EBV) driven leiomyosarcoma tumor for which RNA-seq was not available for assessment of EBV-derived sequences.

FIG. 27 depicts at least a portion of findings by cWGTS with annotation on clinical relevance, according to potential embodiments.

FIGS. 28A-28B depict an analytical validity of cWGTS for clinical biomarkers, according to potential embodiments, a) The left bar plot depicts the proportion of patients with therapy-informing, oncogenic or no relevant findings reported by MSK-IMPACT as defined by OncoKb (Levels 1-4). The right barplot shows the breakdown (0,1,2) of the highest level of OncoKb level in the study cohort, b) Bar plot demonstrating breakdown of highest OncoKb level by number of informative biomarkers in study cohort, c) Barplot demonstrating breakdown of highest OncoKb level by disease class. d) Variant Allele Frequency (VAF) of MSK-IMPACT variants as reported by MSK-IMPACT (x-axis) vs absolute VAF estimates by pileup in WGS data (y-axis). Discrepant mutations are observed along the x-axis. Mutations are color-coded by call status, where Both is called in both assays and ITH is mutations that were not called in higher depth re-sequencing and/or had proportion test p-value<0.05. e) Bar plot demonstrating breakdown of MSK-IMPACT mutations, observed in both WGS and MSK-IMPACT or only MSK-IMPACT (ITH). f) Validation of oncogenic fusions reported by MSK-IMPACT/MSK-Fusion in cWGTS. The asterisk indicates that the SS18-SSX1 that was reported by MSK-Fusion was reported as SS18-SSX2 by RNA-seq and supported by spanning reads in WGS.

FIG. 29 depicts at least a portion of sequencing metrics for mutations (substitutions and indels) reported by MSK-IMPACT and corresponding data in WGS sequencing and validation assays, according to potential embodiments.

FIGS. 30A-30B depict an analytical validity of cWGTS extended, according to potential embodiments, a) Bar plot displaying proportion of patients with OncoKb findings broken down by disease category for the extended pediatric and young adult patient cohort (n=985, median age 10.75, range: 0-39.3) at MSKCC. b) Bar plot displaying overall proportion of patients with OncoKb findings in the extended cohort (n=985). c) Bar plot displaying clonality status (clonal or subclonal), and oncogenic relevance of mutations reported by MSK-IMPACT broken down by whether the mutation was also called by WGS. d) Boxplot displaying WGS effective coverage (derived as purity multiplied by coverage) for samples in study cohort broken down by whether all MSK-IMPACT mutations were called or not. e) Scatter plot showing VAF for mutations called by clinical and/or re-sequenced MSK-IMPACT.

FIG. 31 depicts at least a portion of a summary of germline mutations identified in a cohort, according to potential embodiments.

FIG. 32 depicts at least a portion of fusion events identified by MSK-IMPACT/MSK-FUSION/cWGTS, according to potential embodiments. A value of 0 indicates not assessed, 1: Not called, 2: Supporting evidence.

FIGS. 33A-33B depict a subsampling benchmarking analysis, according to potential embodiments, a) Distribution of median coverages for BAM files at each sub-sampling level. (32 to 100x, 72 to 80x, 97 to 60x and below) b) TMB across coding mutations per sample at each subsampled level. c) Recapitulated, clinically relevant findings at each subsampling level (n=220) color coded by mutation class and labeled with individual ID. Rearrangements and Fusions are ordered by decreasing read support and MSK-IMPACT mutations are ordered by decreasing Variant Allele Fraction.

FIG. 34 depicts at least a portion of median coverages for each down-sampled specimen at each level, according to potential embodiments.

FIGS. 35A-35B depict an assessment of optimal coverage for cWGS, according to potential embodiments, a) Bar plots demonstrating sensitivity of variant detection and 95% confidence intervals by coverage depth (100x, 80x, 60x, 30-40x) from left to right for: 1. Clinically relevant events detected by MSK-IMPACT and cWGTS; 2. Genome-wide SNVs; 3. Genome-wide Indels; and 4. Genome wide SVs. Only data from samples with original median coverage >100x (n=32) are shown. Red dots indicate overall sensitivity of all mutations, b) Histograms of variant allele frequencies for each subsampling level for a representative sample in the study cohort (H135973), showing loss in sensitivity to detect subclonal mutations at lower sequencing depth of coverage c) Scatterplot of effective local coverage vs VAF in sub-sampled BAMs for the clinically relevant calls from MSK-IMPACT. Variants called in sub-sampled BAMs are shown with circles while the missed variants are denoted with X’s. Trendline shows the cumulative binomial distribution for obtaining at least 2 variant reads given the effective coverage and variant allele fraction.

FIGS. 36A-36B depict additional relevant findings detected by cWGTS as compared to standard of care, according to potential embodiments, a) Heatmap of additional relevant findings by cWGTS colored by what technology (WES, WGS, RNA-seq) may detect each event. Columns represent patients, while rows are clinical event types. The asterisks for Germline indicate pathogenicity supported by mutational signatures. b) (top) Stacked bar breakdown of patients with clinically relevant findings by assay. The blue areas (solid or meshed) represent patients with relevant findings from targeted sequencing (RNA and DNA), while the orange areas (solid or meshed) are for patients with findings from cWGTS. The blue/orange mesh indicates patients that had relevant findings from both targeted sequencing and WGTS. (bottom) Stacked bar breakdown of new findings by cWGTS from the patients in the orange section (solid or meshed) from top. The relevant findings are colored by event type.

FIG. 37 depicts at least a portion of somatic SNV/lndel driver mutations in cancer genes identified by WGS, according to potential embodiments.

FIG. 38 depicts fusions identified by cWGTS, according to potential embodiments. Heatmap demonstrating 8 additional fusion genes detected by cWGTS combined (WGS and RNA-seq). Of these, 2 (NUTM1-MGA and SLMAP-NTRK3) were subsequently validated by MSK-Fusion.

FIGS. 39A-39C depict integration of DNA and RNA findings for variant annotation, according to potential embodiments, a) Top panel demonstrates structural variants detected by WGS that result in PAX3-FOXO3 fusion in patient H 134768. Lower panel displays RNA fusion product created by the corresponding genomic SVs displayed validating the event as a functional PAX3-FOXO3 fusion event b) t-SNE clustering of methylation data from rhabdomyosarcoma samples color-coded by disease subtype (ARMS, alveolar; ERMS, embryonal; SCRMS, spindle-cell; and SRMS, sclerosing), supporting the cWGTS finding. The patient harboring the PAX3-FOXO3 fusion clusters with the ARMS samples. c) Top panel demonstrates structural variants resulting in a complex genomic rearrangement targeting chromosomes 6, 8 and 18 resulting in the localization of NFIB enhancer to the MYB locus in patient H133676. Lower panel displays H3K27me3 chromatin marks from Drier et al Nature Genetics 2016. d) Transcripts per million (TPM) expression of MYB across the cohort. The patient with MYB-NFIB event (H133676) is highlighted in orange, demonstrating that the SV event in panels c associates with overexpression of MYB, validating the SV as an enhancer hijacking event. e) Diagram of SV events targeting TP53 gene body in osteosarcoma patients (n=12, the 13th patient’s event breakpoints fall outside of the gene body). SVs are shown as arrows with absolute copy number on y-axis (gray dots) overlayed over the exonic structure of TP53. f) Comparison of TP53 expression in RNA between TP53 rearranged samples and those without any rearrangement demonstrate statistically significant loss (Mann-Whitney U test, p=1.645e-03) in expression in the rearranged samples validating the non-coding TP53 SVs as functional.

FIG. 40 depicts at least a portion of somatic strutural variant driver mutations identified by WGS, according to potential embodiments.

FIG. 41 depicts an example of RNA clustering, according to potential embodiments, t-distributed Stochastic Neighbor Embedding (TSNE) map of RNA expression data from study and extended in-house reference cohort colored by selected disease groups (n=244).

FIG. 42 depicts at least a portion of expression biomarkers identified by RNA using methodology from Horak, P. et al. Comprehensive Genomic and Transcriptomic Analysis for Guiding Therapeutic Decisions in Patients with Rare Cancers. Cancer Discov. (2021) doi:10.1158/2159-8290.CD-21-0126., according to potential embodiments.

FIGS. 43A-43D depict a genome-wide distribution and patterns of somatic mutations for four different patients, according to potential embodiments, a) Neuroblastoma patient (H135421) harboring a pathogenic germline MUTYH variant (p.X297_splice). b) Immature teratoma patient (H135466) with a pathogenic germline PMS2 mutation (p.X180_splice) c) Malignant peripheral nerve sheath tumor patient (H135073) harboring a germline PMS2 variant of unknown significance (VUS) mutation (p.W841*). For each patient, the top panel is a Circos plot showing the different types of somatic mutations along the genome. The outermost ring shows the inter-mutation distance for all SNVs color-coded by the pyrimidine partner of the mutated base. The middle ring shows small insertions (green) and deletions (red). The innermost ring shows copy number changes, and the arcs show SVs. Middle panel is a bar plot showing the absolute number of mutations attributed to the ten mutational signatures with the highest exposure in the tumor. Bottom panel is a bar plot showing the 96 tri-nucleotide contexts of SNVs. d) Genome-wide distribution and patterns of somatic mutations identified in the patient outside the cohort with recurrent osteosarcoma (H201472). WGS results show the sample is hypermutated, with enrichment in SBS26, T>C mutations, repeat mediated deletions, and MSI unstable. Patient was found to be harboring a pathogenic PMS2 variant (p.D699H).

FIGS. 44A-44C depict a telomere length analysis, according to potential embodiments, a) Difference in telomere length between the tumor and matching normal sample as assessed by WGS. Increase in telomere length is shown with green and decrease with red. The individuals are colored by oncogenic events (ATRX: ATRX mutation or SV, MYCN: MYCN amplification, TERT: TERT promoter mutation or SV). b) Telomere length ratio (tumor divided by normal) shown in Neuroblastoma tumor with different oncogenic events that result in aberrant telomere length (n=15). c) Telomere length ratio for Osteosarcoma patients with and without ATRX mutations (n=29). d) Distribution of chromothripsis across disease groups. e) Genes affected by chromothripsis events colored by effect f) Distribution of whole genome duplication across disease groups.

FIGS. 45A-45C depict a genome-wide mutational burden in the context of Immunotherapy, according to potential embodiments, a) Distribution of coding tumor mutational burden (TMB) as assessed by WGS across the cohort (n=114), colored by treatment status of the patient at time of sampling. Dotted line indicates median coding TMB (SNVs and Indels) as previously reported by Grobner et al. Patients are grouped by disease category (NB: Neuroblastoma, CNS: Central nervous system, C: Carcinoma, WT: Wilms tumor, Germ: Germ cell tumor, H: Hepatoblastoma, O: Other). Carcinoma patients C1 and C2 who responded to immunotherapy are labeled. b) Distribution of structural variant (SV) (right) and gene fusion (left) burden across the samples with both WGS and RNA-seq available (n=101). Patient C2 had a poor-quality RNA sample so clonal fusions from another time point from the same patient are shown. c) (top) Genome-wide distribution and patterns of somatic mutations for tumor C1 (H135022), patient with metastatic adrenocortical carcinoma, depicting high SV burden. Circos plots are shown as described in FIGS. 43A-43D. PET imaging shows resolution of a large pulmonary metastatic lesion (red arrow) following treatment with nivolumab and ipilimumab. d) Genome-wide distribution and patterns of somatic mutations for H 135462, a 14 year old with relapsed refractory poorly differentiated clear cell carcinoma with high TMB and SV burden. Circos plots are shown as described in FIGS. 39A-C. PET imaging shows resolution of multiple metastatic lesions (red arrows) following treatment with pembrolizumab.

FIGS. 46A-46B depict genome-wide signals, according to potential embodiments, a) Tumor mutation burden (TMB) assessed by IMPACT (coding mutations), WGS (coding SNVs and Indels divided by exome), WGS genome wide (all SNVs and Indels divided by whole genome). b) Mutational signature contributions per sample. Signatures associated with Platinum and Temozolomide were merged respectively. Treatment status is annotated at the top.

FIG. 47 depicts at least a portion of summary findings of matched cfDNA and FF specimens, according to potential embodiments. An additional genome column (not shown) can have a value of 2 to represent full recapitulation, 1: some recapitulation, 0: no recapitulation.

FIGS. 48A-48C depict a comparison of WGS data from matched fresh frozen tumor tissue and cfDNA, according to potential embodiments, a) Coverage values ordered by estimated tumor context in cfDNA. b) Estimates of tumor content. c) Bar plots showing the proportion of de novo mutation calls in cfDNA that are present in the matched fresh frozen tumor broken down by variant type. cfDNA samples with no high confidence SVs denoted with an asterisk. d) Genome-wide distribution and mutation patterns of matched fresh frozen (left) and cfDNA (right) samples for H158182. Circos plots are shown as described in FIGS. 43A-43D. e) Individual level clonality analysis for H158182. (left) Scatter plot of cancer cell fraction (CCF) values for all substitutions color-coded by the estimated cluster. (middle) Phylogenetic tree representation of clusters annotated with clinically relevant variants. (right) Clone-level mutational signature analysis showing the proportion of mutations attributed to each mutational signature with total numbers of mutations in each cluster shown on the right. Whereas novel drivers associated with these clones could not be determined, cfDNA-specific SNV calls recapitulated mutation signatures in the FF sample, and were enriched for platinum-associated mutational signatures pointing to the existence of therapy-exposed tumor subclones in circulation. Similar to the FIGS. 48A-48C, additional embodiments depicting cfDNA analysis can exist. Such figures could show, for example: Individual level summary comparisons for matched cfDNA and tissue samples in order of purity are used to generate visuals relating to i) Circos plots as described in FIGS. 39A-C, ii) LogR and BAF tumor profiles from cgpBattenberg, iii) individual level phylogenetic tree or tissue phylogenetic tree if no cfDNA subclones (H156407, H136631) with clinically relevant variants annotated, iv) bar plot of clusters estimated by substitutions with percentage of variants with pileup support from cfDNA shaded and annotated, v) mutational signature exposures per same clusters, vi) 96 mutation contexts for each cluster.

FIG. 49 provides an example of prioritized allelic imbalances for a patient with loss of MTAP and homozygous loss of CDKN2A/B, according to various potential embodiments. Total copy number is shown in the cells on the left while fold change in RNA expression is shown on the right.

FIG. 50 depicts an example of a cohort comparison for small mutations, according to various potential embodiments. The selected patient is shown with circles and is overlaid on a violin distribution for a selected cohort. The patient has a relatively high number of mutations compared to the cohort.

FIG. 51 illustrates a flow diagram of an example process for performing a cWGTS analysis, according to potential embodiments.

### DETAILED DESCRIPTION

The disclosed technology provides an unconventional approach that integrates particular RNA analyses with particular DNA analyses in a computationally-efficient manner that improves the detection, and correct classification, of diseases and their characteristics within a clinically-relevant timeframe (e.g., days instead of weeks). As examples, the disclosed approach has been demonstrated to: provide more accurate detection of indeterminate tumors (e.g., Case Study 1 and Case Study 8 discussed herein); identify a difficult-to-interpret complex genomic rearrangement resulting in a particular fusion (e.g., Case Study 2); identify a new partner gene, thereby clarifying a diagnosis (e.g., Case Study 3); better evaluate the aggressiveness or prognosis of tumors (e.g., Case Study 4 and Case Study 5); elucidate oncogenic nature of a tumor (e.g., Case Study 6); detect rare cancer genes not covered by panel based tests (e.g., Case Study 9); and detect germline events in rare hereditary genes that may not be sequenced by targeted panels (e.g., Case Study 10).

It is to be appreciated that certain aspects, modes, embodiments, variations and features of the present methods are described below in various levels of detail in order to provide a substantial understanding of the present technology. It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, the terms “subject”, “patient”, or “individual” can be an individual organism, a vertebrate, a mammal, or a human. In some embodiments, the subject, patient or individual is a human.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

Embodiments discussed in the present disclosure are directed towards systems and methods for determining anti-cancer therapy and/or treatment, for example, using cancer whole genome and transcriptome sequencing (cWGTS). Cancer can be caused by the accumulation of somatic variants (e.g., point mutations, structural variants (SVs) and copy number alterations (CNAs)) that drive oncogenesis, disease progression, and/or define therapeutic vulnerabilities. The introduction of next-generation sequencing (NGS)-based targeted gene panel assays can aid in disease diagnosis and guided care, as well as improve patient outcomes through refinement of treatment options. However, targeted panels are optimized to assess clinical biomarkers in common cancers. For patients with pediatric or rare cancers that have low mutation burden and are primarily driven by structural variants and fusion genes, for example, panel tests may fail to identify a clinical biomarker (in most cases). Said failure to identify a clinical biomarker can underscore an unmet need for better diagnostic workflows to guide clinical management.

Embodiments of the systems and methods described herein use cWGTS to enable the assessment of the full spectrum of germline and somatically acquired mutations, SVs, and/or CNAs, along with quantification of tumor mutation burden (TMB) and genome-wide mutational patterns. The likely clinical utility of cWGTS in pediatric and rare cancers, for instance, is increasingly evidenced. However, clinical implementations of WGS can be challenged by cost of sequencing, complexity of laboratory and analytical workflows to process large scale data within clinically relevant timeframes, concerns about the sensitivity of low coverage WGS in detecting actionable mutations captured by high-depth panel assays, and/or the interpretability of cWGTS findings with regards to clinical utility. The systems and methods presented herein demonstrate the feasibility, analytical validity, and/or critical technical considerations for the implementation of cWGTS in cancer care.

### Systems for Performing a cWGTS Analysis

### i. Computing and Network Environment

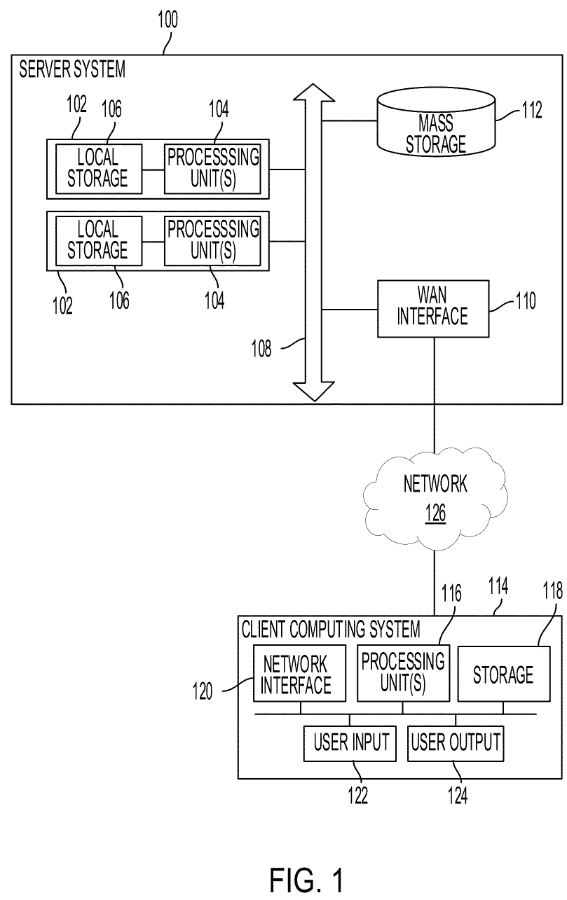

Various operations described herein can be implemented on computer systems. FIG. 1 shows a simplified block diagram of a representative server system 100, client computer system 114, and network 126 usable to implement certain embodiments of the present disclosure. In various embodiments, server system 100 or similar systems can implement services or servers described herein or portions thereof. Client computer system 114 or similar systems can implement clients described herein. The system 300 described herein can be similar to the server system 100. Server system 100 can have a modular design that incorporates a number of modules 102 (e.g., blades in a blade server embodiment); while two modules 102 are shown, any number can be provided. Each module 102 can include processing unit(s) 104 and local storage 106.

Processing unit(s) 104 can include a single processor, which can have one or more cores, or multiple processors. In some embodiments, processing unit(s) 104 can include a general-purpose primary processor as well as one or more special-purpose coprocessors such as graphics processors, digital signal processors, or the like. In some embodiments, some or all processing units 104 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 104 can execute instructions stored in local storage 106. Any type of processors in any combination can be included in processing unit(s) 104.

Local storage 106 can include volatile storage media (e.g., DRAM, SRAM, SDRAM, or the like) and/or non-volatile storage media (e.g., magnetic or optical disk, flash memory, or the like). Storage media incorporated in local storage 106 can be fixed, removable or upgradeable as desired. Local storage 106 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 104 need at runtime. The ROM can store static data and instructions that are needed by processing unit(s) 104. The permanent storage device can be a non-volatile read-and-write memory device that can store instructions and data even when module 102 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 106 can store one or more software programs to be executed by processing unit(s) 104, such as an operating system and/or programs implementing various server functions such as functions of any system described herein, or any other server(s) associated therewith.

“Software” refers generally to sequences of instructions that, when executed by processing unit(s) 104 cause server system 100 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 104. Software can be implemented as a single program or a collection of separate programs or program modules that interact as desired. From local storage 106 (or non-local storage described below), processing unit(s) 104 can retrieve program instructions to execute and data to process in order to execute various operations described above.

In some server systems 100, multiple modules 102 can be interconnected via a bus or other interconnect 108, forming a local area network that supports communication between modules 102 and other components of server system 100. Interconnect 108 can be implemented using various technologies including server racks, hubs, routers, etc.

A wide area network (WAN) interface 110 can provide data communication capability between the local area network (interconnect 108) and the network 126, such as the Internet. Technologies can be used, including wired (e.g., Ethernet, IEEE 802.3 standards) and/or wireless technologies (e.g., Wi-Fi, IEEE 802.11 standards).

In some embodiments, local storage 106 is intended to provide working memory for processing unit(s) 104, providing fast access to programs and/or data to be processed while reducing traffic on interconnect 108. Storage for larger quantities of data can be provided on the local area network by one or more mass storage subsystems 112 that can be connected to interconnect 108. Mass storage subsystem 112 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 112. In some embodiments, additional data storage resources may be accessible via WAN interface 110 (potentially with increased latency).

Server system 100 can operate in response to requests received via WAN interface 110. For example, one of modules 102 can implement a supervisory function and assign discrete tasks to other modules 102 in response to received requests. Work allocation techniques can be used. As requests are processed, results can be returned to the requester via WAN interface 110. Such operation can generally be automated. Further, in some embodiments, WAN interface 110 can connect multiple server systems 100 to each other, providing scalable systems capable of managing high volumes of activity. Other techniques for managing server systems and server farms (collections of server systems that cooperate) can be used, including dynamic resource allocation and reallocation.

Server system 100 can interact with various user-owned or user-operated devices via a wide-area network such as the Internet. An example of a user-operated device is shown in FIG. 1 as client computing system 114. Client computing system 114 can be implemented, for example, as a consumer device such as a smartphone, other mobile phone, tablet computer, wearable computing device (e.g., smart watch, eyeglasses), desktop computer, laptop computer, and so on.

For example, client computing system 114 can communicate via WAN interface 110. Client computing system 114 can include computer components such as processing unit(s) 116, storage device 118, network interface 120, user input device 122, and user output device 124. Client computing system 114 can be a computing device implemented in a variety of form factors, such as a desktop computer, laptop computer, tablet computer, smartphone, other mobile computing device, wearable computing device, or the like.

Processor 116 and storage device 118 can be similar to processing unit(s) 104 and local storage 106 described above. Suitable devices can be selected based on the demands to be placed on client computing system 114; for example, client computing system 114 can be implemented as a “thin” client with limited processing capability or as a high-powered computing device. Client computing system 114 can be provisioned with program code executable by processing unit(s) 116 to enable various interactions with server system 100.

Network interface 120 can provide a connection to the network 126, such as a wide area network (e.g., the Internet) to which WAN interface 110 of server system 100 is also connected. In various embodiments, network interface 120 can include a wired interface (e.g., Ethernet) and/or a wireless interface implementing various RF data communication standards such as Wi-Fi, Bluetooth, or cellular data network standards (e.g., 3G, 4G, LTE, etc.).

User input device 122 can include any device (or devices) via which a user can provide signals to client computing system 114; client computing system 114 can interpret the signals as indicative of particular user requests or information. In various embodiments, user input device 122 can include any or all of a keyboard, touch pad, touch screen, mouse or other pointing device, scroll wheel, click wheel, dial, button, switch, keypad, microphone, and so on.

User output device 124 can include any device via which client computing system 114 can provide information to a user. For example, user output device 124 can include a display to display images generated by or delivered to client computing system 114. The display can incorporate various image generation technologies, e.g., a liquid crystal display (LCD), light-emitting diode (LED) including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that function as both input and output device. In some embodiments, other user output devices 124 can be provided in addition to or instead of a display. Examples include indicator lights, speakers, tactile “display” devices, printers, and so on.

Some embodiments include electronic components, such as microprocessors, storage and memory that store computer program instructions in a computer readable storage medium. Many of the features described in this specification can be implemented as processes that are specified as a set of program instructions encoded on a computer readable storage medium. When these program instructions are executed by one or more processing units, they cause the processing unit(s) to perform various operation indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 104 and 116 can provide various functionality for server system 100 and client computing system 114, including any of the functionality described herein as being performed by a server or client, or other functionality.

It will be appreciated that server system 100 and client computing system 114 are illustrative and that variations and modifications are possible. Computer systems used in connection with embodiments of the present disclosure can have other capabilities not specifically described here. Further, while server system 100 and client computing system 114 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

Aspects of the operating environments and components described above will become apparent in the context of the systems and methods disclosed herein.

### cWGTS Analysis from Tumor Biopsies and Normal Control

In certain embodiments, a cWGTS analysis of tumor samples and healthy samples can be performed based on a plurality of steps (e.g., Step I, Step II, Step III, and/or other steps), according to the systems and methods described herein. One or more inputs to the cWGTS analysis can include or correspond to a WGS and/or RNA-seq data generated from tumor biopsies/samples, such as fresh frozen DNA, formalin-fixed paraffin-embedded tissue (FFPE), circulating free DNA, and/or cerebral spinal fluid. In certain embodiments, WGS data of normal control biopsies (e.g., healthy samples) can be from peripheral blood, nails, a skin biopsy, and/or adjacent normal tissue.

### ii. Step I: Sample and Data Quality Control

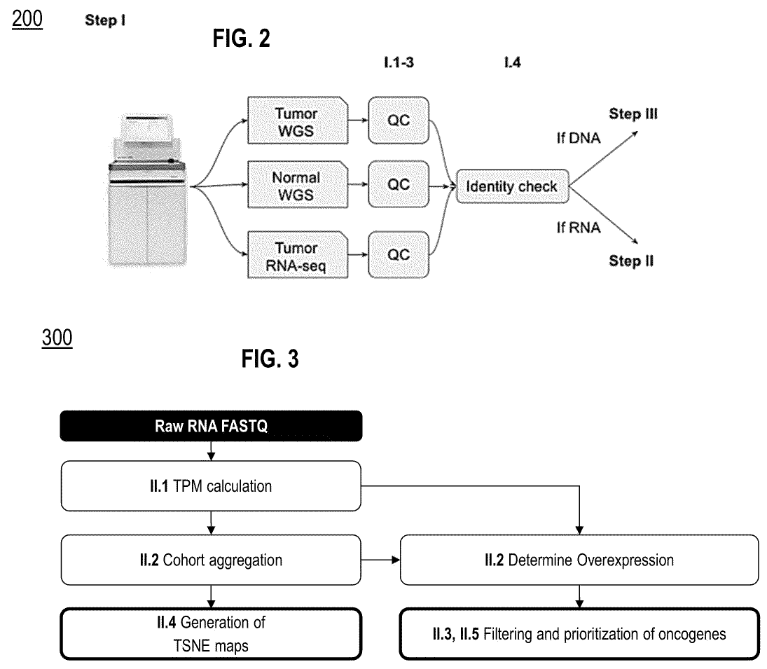

Upon completing a sequencing, data can be imported and aligned against the human reference genome to produce a plurality of input files. The plurality of input files may include a WGS of the tumor-derived DNA (e.g., at 80x to 110x coverage, such as at 100x coverage), a WGS of normal tissue-derived DNA (e.g., at 40 x to 70x coverage, such as at 60x coverage), and/or a whole transcriptome sequencing (WTS) of tumor-derived RNA (e.g., 40 to 80 million reads, such as 60 million reads). An output of Step I can include an indication of an attainment of pass criteria in steps I.1 to I.4. Attainment of said pass criteria can initiate the next stage of the process (e.g., variant calling). FIG. 2 depicts a diagram of an example process as described in Step I.

### Step I.1

Step 1.1 can include performing quality controls for data quality and median coverage (e.g., more than about 70% of genome covered, such as 70% to 80% of genome covered). Certain approaches for calculating genome-wide sequencing coverage, such as Mosdepth, can be used with a mapping quality threshold of, for example, zero to 20 phred (e.g., 20 phred or other thresholds) to calculate a high-quality depth for DNA bam files (or other types of DNA files). A median coverage in the normal and tumor may be 20x -30x and 50x to 60x, respectively, such as at least 30x and 60x, respectively. Certain approaches for detecting potential problems in a high-throughput sequencing dataset, such as Fastqc (https://github.com/s-andrews/FastQC), may be used for both DNA and RNA fastqs. Approaches for post-sequencing quality control of RNA, such as RNA-SeQC (https://github.com/getzlab/rnaseqc), can be used just for RNA bam files.

### Step I.2

Step I.2 can include a concordance genotyping of individually derived data (e.g., tumor WGS, tumor RNA and normal/healthy WGS) across, for example, at least 5000 SNP markers, or at least 7000 SNP markers, with a population minor allele frequency of 40% to 60%, or greater than 50%, using Conpair (or other approaches, such as Somalier (https://github.com/brentp/somalier), for detection of sample swaps and cross-individual contamination in whole-genome and whole-exome tumor-normal sequencing experiments), to ensure that the data files are associated with the same subject. Unmatched samples may be flagged and/or removed from the process.

### Step I.3

Step I.3 can include an estimation of inter-sample contamination (e.g., cross-individual contamination from another individual getting mixed in) in the data. Contaminated samples may be flagged and/or removed from subsequent analysis.

### Step I.4

Step I.4 can include an estimation of tumor cell content (e.g., tumor purity) in the DNA derived using the Battenberg algorithm (or other algorithms for whole genome sequencing subclonal copy number, such as Hatchet (https://github.com/raphael-group/hatchet)). A 15% to 30% tumor purity (e.g., a 20% tumor purity or other tumor purities) may be required for successful execution of the analytics workflow.

### iii. Step II: Gene Expression Analysis

One or more inputs to Step II can include or correspond to a raw whole transcriptome sequencing of tumor derived RNA (e.g., FASTQ), a proprietary cohort level database (feather.db) containing TPM values for clinically annotated tumor derived RNA-seq files (e.g., Appendix 3), and/or a BED file (or other files such as GTF format files) containing genomic coordinates of cancer genes (e.g., cancer genes from OncoKb, as seen in Appendix 4). One or more outputs of Step II can include a list of priority aberrantly expressed genes (based on clinical or biological relevance), alongside gene expression metrics (TPM) and log fold change relative to reference cohort. In certain embodiments, the one or more outputs can include a tSNE map of an index case, alongside cohort.db. FIG. 3 depicts a diagram of an example process as described in Step II, in which a workflow summarizing a process for deriving gene expression-based metrics is discussed.

### Step II.1

Step II.1 can include or correspond to a deployment and/or application of certain tools, such as SALMON (v0.10.0, https://github.com/COMBINE-lab/salmon), to estimate transcript per million (TPM) values for each cancer gene. For all genes in the human transcriptome, gene-level TPM can be calculated by summing TPM values for each gene.

### Step II.2

Step II.2 can include or correspond to a detection and/or identification of over-expressed and/or under-expressed genes. Over-expressed and/or under-expressed genes can be flagged when a patient’s gene TPM is outside the 95% (or other percentage values) confidence interval of the cohort mean/std, and/or the fold change from the cohort mean is greater or lower than 2.

### Step II.3

In Step II.3, gene identifiers can be intersected with known cancer genes (e.g., from OncoKb) to identify established cancer genes with aberrant gene expression that target established cancer genes.

### Step II.4

Step II.4 can include a generation of t-distributed stochastic neighbor embedding (tSNE) maps of index cases alongside data in reference db (see FIG. 3). The Cohort Feather DB (or other databases) can be used to generate tSNE maps using Log(TPM +1) values.

### Step II.5

Step II.5 can include a prioritization (e.g., hierarchy-based ranking) of aberrantly expressed genes on the basis of an intersection of aberrantly expressed genes with established cancer genes (e.g., listed in decreasing expression fold change) and/or potential targeted treatments (e.g. GPC3). Potential targeted treatments can be highlighted using a combination of manually curated literature and/or available clinical trials.

### Application Embodiment 1

A tumor type profile can be derived by clustering against a sample tumor gene expression profile alongside a reference cohort of known clinical entities. Said clustering can enable resolution in tumors that are difficult to diagnose (e.g., according to a standard-of-care assessment). An example of this application is the subject of Case Study 1, who had an indeterminate diagnosis of neuroblastoma metastasis. However, WGS analysis identified a pathognomonic schwannoma fusion gene, suggesting a different diagnosis in said subject. A tSNE analysis of RNA shows that the tumor is closest to another schwannoma (e.g., instead of to neuroblastomas) in the extended cohort, thereby confirming the schwannoma diagnosis.

### Application Embodiment 2

Non-canonical fusion genes can be accessed to see if they cause similar expression patterns to their canonical counterparts and can be treated as such (e.g., see Case Study 2 and Case Study 3). One example of this application is Case Study 2, in which a WGS analysis identified a difficult-to-interpret complex genomic rearrangement resulting in an SH3PXD2A-HTRA1 fusion. In a tSNE analysis, the tumor clustered with another tumor harboring a simpler genomic event leading to the same fusion gene, thereby confirming the oncogenic nature of the complex fusion. Another example of this application is Case Study 3, in which a new partner gene (e.g., FOXO3) is identified for PAX3, thereby clarifying the diagnosis of alveolar rhabdomyosarcoma. In a tSNE analysis, the tumor clusters with other tumors with the canonical fusion (e.g., PAX3-FOXO1 and PAX7-FOXO1).

### iv. Step III: Estimation of Ploidy and Detection of Allelic Imbalances

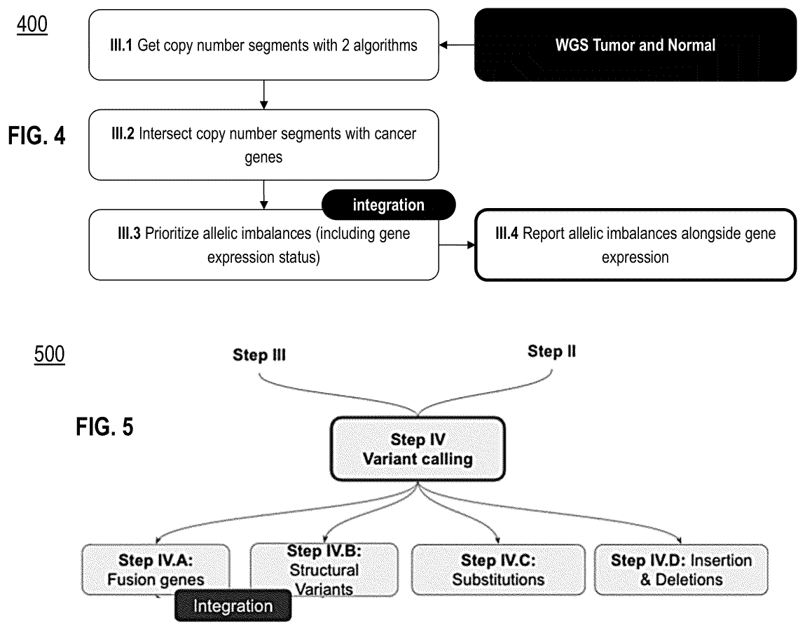

One or more inputs to Step III can include or correspond to an aligned tumor and/or normal BAM files (or other files) that pass (e.g., fulfill) the QC criteria of Step I. One or more inputs to Step III can include a BED file (or other files) containing genomic coordinates of cancer genes from OncoKb, for example (see Appendix 3). One or more outputs of Step III can include a file containing prioritized allelic imbalances for reporting by the analytics workflow. FIG. 4 depicts a diagram of an example process as described in Step III.

### Step III.1

Step III.1 can include using two algorithms (e.g., Battenberg (https://github.com/cancerit/cgpBattenberg) and/or BRASS (v4.0.5 with GRASS v1.1.6; https://github.com/cancerit/BRASS)) for the detection of allelic imbalances (e.g., deletions, loss of heterozygosity, and/or amplifications) to estimate LogR and aberrant B-allele fractions, in order to detect high confidence aberrant copy number segments from the input data (for a particular application).

### Step III.2

Step III.2 can include intersecting aberrant copy number segments with genomic coordinates of established cancer genes. Aberrant copy number segments are annotated to include a number of copies in the segment. For example, 0 or 1 copies may indicate a deletion, 2+0 can indicate a region of copy neutral loss of heterozygosity, and/or segments with >2 copies can indicate an amplification. Losses may be reported by one total copy if the gene is a predicted haploinsufficient gene (e.g., Appendix 6) or zero total copies otherwise. Genomic coordinates for allelic imbalances can be intersected with the known cancer gene BED file to identify regions that target established cancer genes. Aberrant segments may be annotated with the cancer genes that are affected within the segment or segment boundary.

### Step III.3

Step III.3 can include conducting and/or performing a prioritization of allelic imbalances using a set of criteria (e.g., hierarchical ranking). The set of criteria may include a genome wide ploidy status of diagnostic importance, such as whole genome duplication (WGD). The set of criteria may include that the aberrant segment overlaps with known cancer genes. The set of criteria may include that the aberrant segment direction (e.g., deletion, loss of heterozygosity, and/or amplification) is consistent with the established function of a cancer gene. For example, the established function of the cancer gene can include deletions or loss of heterozygosity in tumor suppressors (e.g., TP53) or amplification in oncogenes (e.g., MYC). For aberrant copy number segments that target known cancer genes (e.g., per reference list), integration of corresponding gene expression metrics for the genes can be evaluated to provide further supporting evidence on the event. For example, gene amplifications associated with an increase in gene expression and/or losses of tumor suppressor genes may be associated with a decreased gene expression.

### Step III.4

Step III.4 can include reporting and/or providing prioritized allelic imbalances (e.g., from Step III.3) and/or the associated gene expression metrics for the affected genes.

### Application Embodiment 1

In one example, RNA and DNA can be integrated. Integration of RNA and DNA may enable cross validation of cryptic rearrangements affecting oncogenic loci. In one example (e.g., Case Study 4) a subject is diagnosed with desmoid-type fibromatosis, a benign tumor that is rarely malignant. The cWTGS analysis can identify a complex TERT event resulting in over-expression. Certain mutations, such as TERT promoter mutations, and/or TERT over-expression may correlate with poor prognosis in a plurality of tumor types. Genomic rearrangements of TERT, although comparatively rare, may lead to TERT over-expression. Said complex TERT event may explain the aggressive nature of said tumor.

### v. Step IV: Somatic Variant Calling

Step IV can include detecting, prioritizing, and/or reporting the presence of clinically relevant somatic variants across mutation classes to include RNA fusion genes (Step IV.A), structural variants (Step IV.B), insertions and deletions (indels) (Step IV.C) and/or single nucleotide variations (SNVs) (Step IV.D). FIG. 5 depicts a diagram of an example process as described in Step IV.

### Step IV.A: Detection of RNA Fusions

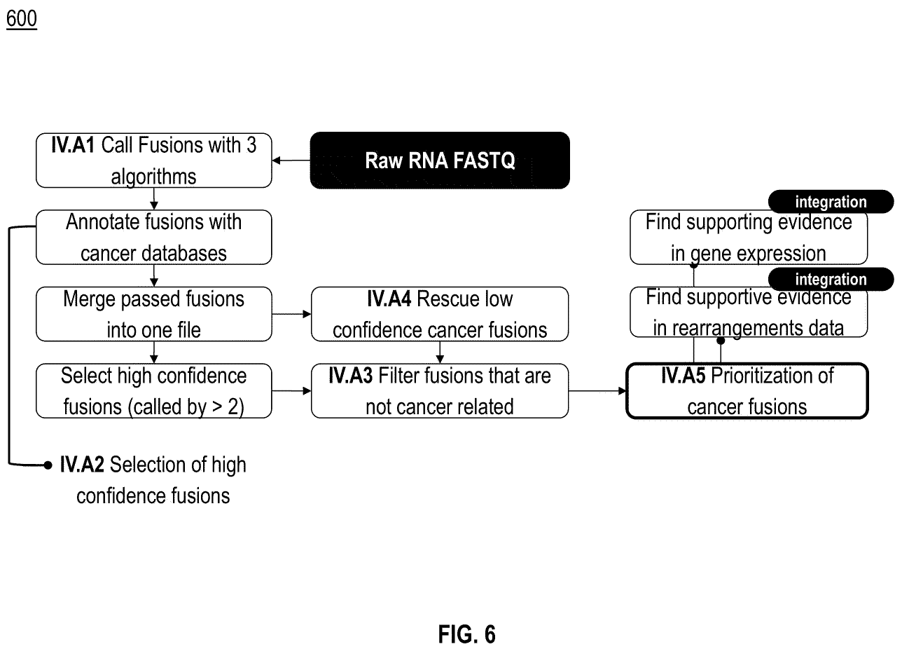

One or more inputs to Step IV.A can include an Illumina tumor paired end RNA seq. and/or one or more databases of candidate fusion genes (such as databases of FusionCatcher (https://github.com/ndaniel/fusioncatcher/blob/master/doc/manual.md#62---output-data-output-data)). FIG. 6 depicts a diagram of an example process as described in Step IV.A (e.g., high confidence fusions).

### (i) Step IV.A1

Step IV.A1 may include deploying and/or applying at least three independent fusion gene callers on raw data. Each independent fusion gene caller can be executed post processing to retrieve high-confidence passed Variant Call Files (VCF) and/or other types of files. The at least three independent fusion gene callers may include FusionCatcher (v1.0.0; https://github.com/ndaniel/fusioncatcher), STAR-Fusion (v1.3.1; https://github.com/STAR-Fusion/STAR-Fusion), and Fu-Seq (v1.1.1; https://github.com/nghiavtr/FuSeq).

### (ii) Step IV.A2

Step IV.A2 may include selecting high-confidence fusion genes (based on, for example, orthogonal algorithm detection and read support). In some embodiments, high-confidence passed VCF files may be annotated. For an output of each fusion gene caller, and for each passed call, Step IV.A2 may include annotating the genes and gene pairs implicated in each call genewise (e.g., are either of the genes in the Cancer Gene Census or lincRNA) and/or gene pair-wise (e.g., are the genes next to each other or implicated in a known fusion together), using multiple reference databases (e.g., >30 reference databases) of previously reported fusion genes (e.g., provided by FusionCatcher, see Appendix 2). In some embodiments, Step IV.A2 may include merging and/or combining passed calls (e.g., based on gene pairs and/or other information) into a single file, and/or filtering merged calls for high confidence variants (regardless of reported breakpoints, e.g., entries for the same pair of genes from one or more callers are grouped together). Step IV.A2 may include selecting and/or identifying passed high confidence calls detected by at least two independent callers (i.e., calls that are detected by two independent callers may be deemed high confidence calls).

### (iii) Step IV.A3

Step IV.A3 may include filtering fusion genes according to one or more criteria. The one or more criteria may include that a fusion is not present in FusionCatcher databases (or other databases), which provides aggregates from many external databases, listing fusions in normal healthy tissues (e.g., gtex, 1000 genomes, non-cancer tissues). The FusionCatcher databases may include at least one of: “healthy” (e.g., healthy tissues), “conjoing” (e.g., known conjoined genes), “hpa” (e.g., healthy tissues), “banned”, (e.g., known artifacts), “gtex” (e.g., healthy tissues), “paralogs” (e.g., paralog genes), “1000 genomes” (e.g., healthy tissues), “readthrough” (e.g., readthrough genes), “mt” (e.g., mitochondria), “cacg” (e.g., known conjoined genes), “ensembl_partially_overlapping” (e.g., overlapping genes), “non_cancer_tissues” (e.g., healthy tissues), or “ensembl_fully_overlapping” (e.g., overlapping genes). In certain embodiments, the one or more criteria may include that a fusion is absent from recurrent artifacts databases (e.g., FusionCatcher). In some embodiments, the one or more criteria may include that a fusion does not represent known paralogue genes, read through, conjoined, and/or overlapping genes per established databases (e.g., Ensembl).

### (iv) Step IV.A4

Step IV.A4 may include a rescue process. The rescue process can recover fusion genes that were detected in Step IV.A2 but were not passed by at least two independent variant callers (e.g., using FusionCatcher DB as a reference for known cancer genes). The rescue process may be used to detect lowly expressed fusions in established cancer genes. In some embodiments, the rescued fusions can be subsequently filtered through the same process as in Step IV.A3, wherein the rescued fusions can have at least 1 spanning read.

### (v) Step IV.A5

Step IV.A5 may include prioritization of high confidence calls for reporting. Said prioritization of high confidence calls may use the following criteria (e.g., in a predetermined sequence, such as from A5.1 to A5.6):
A5.1: Known oncogenic fusions from FusionCatcher db (or other databases).
A5.2: The fusion call can be predicted as oncogenic by OncoKb db (or other databases).
A5.3: Supporting evidence of a call in DNA rearrangement calls (e.g., Step IV.B) to include, aberrant read pairs, spanning reads (e.g., PASSED calls or at least 5 total reads, spanning or split, between the two genes).
A5.4: Supporting evidence of aberrant gene expression for either gene participating in the fusion call (e.g., Step II).
A5.5: Viral integration as a part of Fusioncatcher can be used to identify virus-associated cancers, such as EBV in Leiomyosarcoma.
A5.6: Total read support (e.g., a sum of spanning and split reads), in descending order

### Step IV.B: Detection of Somatic Structural Variants

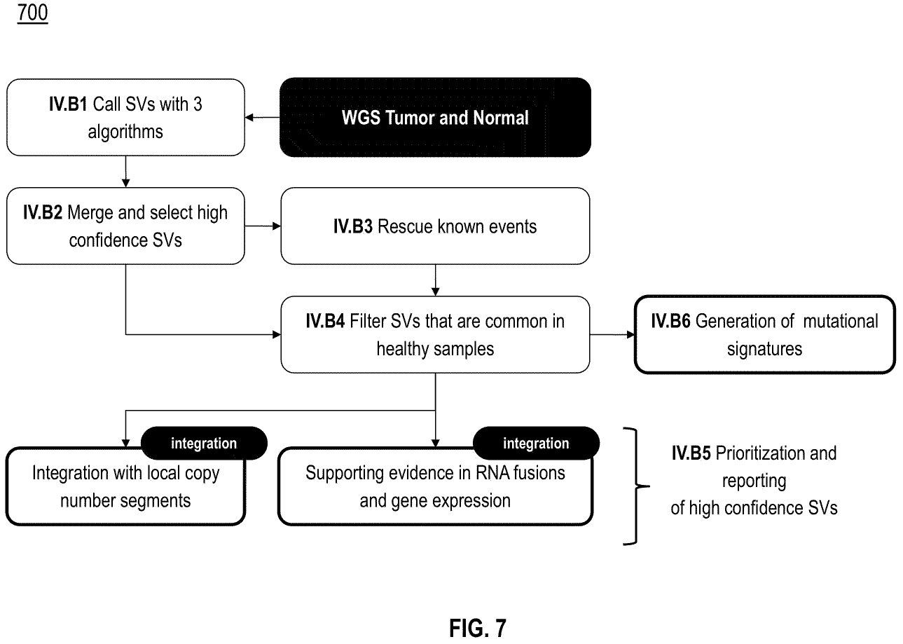

One or more inputs to Step IV.B can include an aligned matched tumor and/or normal BAM files (or other files). In some embodiments, the one or more inputs to Step IV.B can include structural variant calls in a reference panel of >98 normal tissue controls (or other numbers of normal tissue controls). In certain embodiments, the one or more inputs to Step IV.B can include structural variant calls in reference databases (e.g., 1000 genomes project). FIG. 7 depicts a diagram of an example process as described in Step IV.B (e.g., high confidence structural variants (SVs)).

### (i) Step IV.B1

Step IV.B1 may include deploying and/or applying one or more independent structural variant callers on raw data. Each independent structural variant caller can be executed post processing to retrieve high-confidence passed Variant Call Files (VCF) and/or other types of files. The one or more independent structural variant callers may include SvABA (~v1.0.0 commit 47c7a88; https://github.com/walaj/svaba), BRASS (v4.0.5 with GRASS v1.1.6; https://github.com/cancerit/BRASS), and/or GRIDSS (v2.2.2; https://github.com/PapenfussLab/gridss).

### (ii) Step IV.B2

Step IV.B2 may include selecting high-confidence structural variant calls. In some embodiments, calls greater than 600 bp may be merged/combined by, for example, a 50 bp to 100 bp window, such as a 50 bp window, a 75 bp window, or a 100 bp window (or other windows) around breakpoints and/or a same orientation keeping the median breakpoint. Certain approaches for annotating structural variants and/or the genes hit by them, such as ANNOTSV (https://lbgi.fr/AnnotSV), can be used to annotate merged calls. Structural variant calls that have passed filtering criteria from at least two independent structural variant callers can be selected. For example, final high confidence may be calls after merging which are >600 bp and called by at least 2 callers.

### (iii) Step IV.B3

Step IV.B3 may include rescuing oncogenic fusion genes that are difficult and/or challenging to map (e.g., for being highly repetitive or multimapping). For example, a list of fusion genes, known to localize within regions that are challenging-to-map (e.g., high GC content, IGH, CIC, and/or DUX4), may be retrieved using a customized workflow. Said customized workflow may search and/or analyze raw VCF calls from each fusion caller (e.g., SvABA) for reads supporting the fusion. Events supported by at least three aberrant read pairs, targeting both genes in a pair associated with an established (e.g., reference database) fusion gene, can be flagged and/or embedded in a rescue list (e.g., flagged for manual curation). Aberrant read pairs targeting established non-coding events (e.g., TERT) can be retrieved by performing a single-region search with a large bp window from the confident calls list corresponding to known event lists (e.g., section H3).

### (iv) Step IV.B4

Step IV.B4 may include filtering high-confidence structural variant calls. The high-confidence structural variant calls may be filtered according to (or based on) at least one or more criteria. The at least one or more criteria may include that structural variant calls are not present in the control panel of 98 normal samples (or other number of samples) analyzed by the analytics workflow. In certain embodiments, the at least one or more criteria may include that structural variant calls are not present in normal control reference databases (e.g., 1000 genomes and gnomAD databases).

### (v) Step IV.B5

Step IV.B5 may include prioritization of high confidence calls for reporting. Said prioritization of high confidence calls may use the following criteria (e.g., in a predetermined sequence, such as from B5.1 to B5.5 – this may be a hierarchical sorting, and may be reordered if relative importance are changed after consideration):
B5.1: Structural variant can target known cancer genes per OncoKb db (or other databases).
B5.2: Structural variant calls may align to an aberrant copy number segment as determined by Step III.
B5.3: Structural variant may have supporting evidence in RNA fusion calls (e.g., Step IV.A).
B5.4: Supporting evidence of aberrant gene expression of genes participating in structural variant calls (e.g., Step II).
B5.5: Chromothripsis can be detected using approaches to detect chromothripsis events from NGS data, such as ShatterSeek (https://github.com/parklab/ShatterSeek). Chromothripsis can be detected with copy number segments from Step III annotated with gene information.

### (vi) Step IV.B6

Step IV.B6 can include ascertaining SV signatures during post-processing. Certain clustering approaches, such as ClusterSV (https://github.com/cancerit/ClusterSV), can be used to identify first structural variant clusters. Clusters may be split and/or divided if they are not within, for example, 20 kbps (or other numbers of kbps) of each other. If ClusterSV, for example, fails to provide clusters, one or more clusters can be created if the events are within, for example, 20 kbps (or other numbers of kbps). The clusters can be classified according to the following logic/criteria:
Singletons can be tandem duplication, simple deletion, inversion and/or unbalanced translocation.
If there are 2 events, verify whether said events are a reciprocal inversion or a reciprocal translocation.
If there are more than 2 events, call and/or use the cluster complex.

### (vii) Application Embodiment 1

One example embodiment may include or correspond to a detection and/or validation of diagnostic fusion genes. Case studies 1-3 and 5 illustrate such example embodiments, in which oncogenic fusion is identified and cross-validated in both RNAseq and WGS.

### (viii) Application Embodiment 2

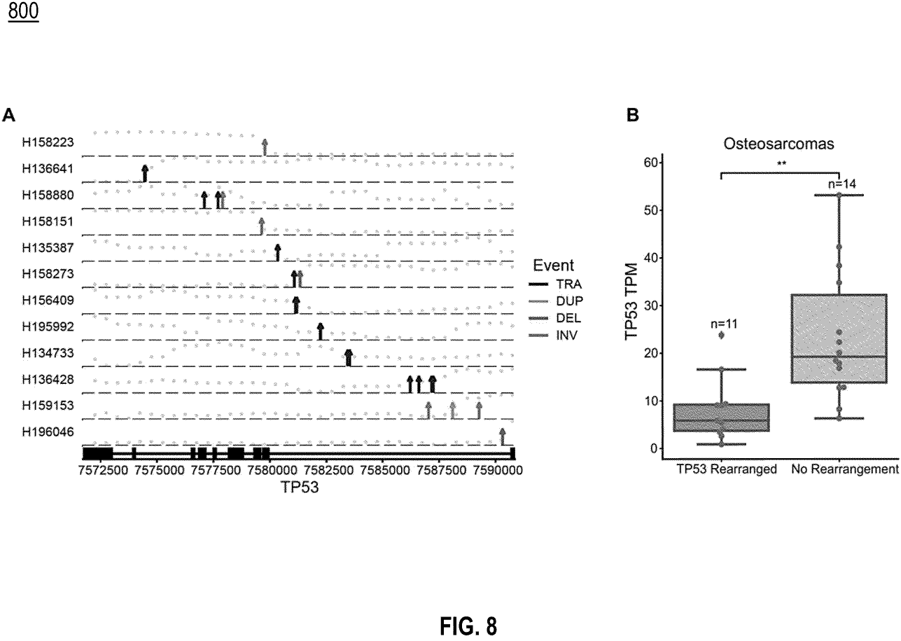

One example embodiment may include or correspond to a validation of functional structural variants targeting intronic regions of established cancer genes. In certain embodiments, an example application may include or correspond to rearrangements affecting a TP53 locus, which can result in down-regulation of the gene compared to tumors without any such rearrangements. For instance, FIG. 8 depicts a plurality of high-confidence structural variants affecting the TP53 locus. In panel A of FIG. 8, each lane identifies an independent sample. The arrows demonstrate localization of SV’s (e.g., TRA: Translocation, DUP: Duplication, DEL: Deletion, INV: Inversion) targeting non-coding regions of the TP53 locus (e.g., x-axis). Panel B of FIG. 8 depicts a significantly lower relative gene expression for TP53 (TPM) for patients with SV’s (e.g., “rearranged”), as compared to patient samples with no TP53 mutations (e.g., “no rearrangement”). As such, SV calls can be integrated to gene level expression to validate and/or prioritize putative cancer gene mutations for reporting.

### (ix) Application Embodiment 3

One example embodiment may include a validation of functional structural variants targeting regulatory regions of established cancer genes. One example application may include or correspond to Case Study 6, in which integration of RNAseq and WGS clarifies the oncogenic nature of the complex genomic rearrangement affect MYB and NFIB.

### Step IV.C: Detection of Somatic Substitutions

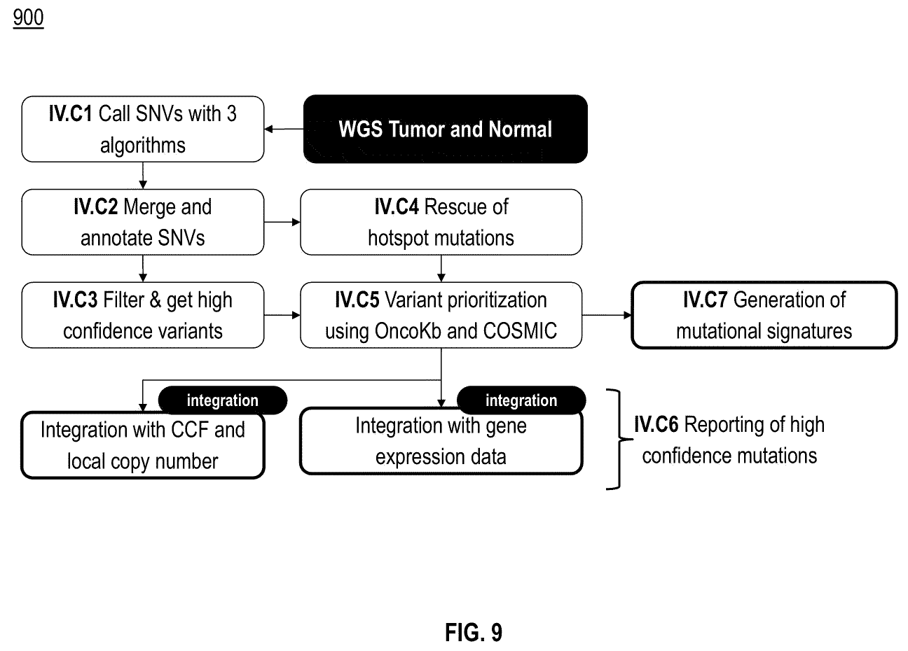

One or more inputs to Step IV.C can include an aligned matched tumor and/or normal BAM files (or other types of files). In some embodiments, the one or more inputs can include ploidy and segmentation for allelic imbalances from Step III, and/or a BED file of known cancer genes and hotspot mutations from OncoKB. In some embodiments, the one or more inputs can include a BED file of germline indel calls generated by cgpPindel and/or other calling algorithms. In some embodiments, the one or more inputs can include substitution calls in a reference panel of >98 normal tissue controls (or other numbers of normal tissue controls) analyzed by the analytics workflow. In some embodiments, the one or more inputs can include a utilization of reference data from public databases. The public databases may comprise the Ensembl database Variant Effect Predictor (v92, https://github.com/Ensembl/ensembl-vep), the Variant annotation Generator VAGrENT (v3.3.0, https://github.com/cancerit/VAGrENT), the genome aggregator database gnomAD, the Catalogue of Somatic mutations in cancer COSMIC, and/or OncoKb. FIG. 9 depicts a diagram of an example process as described in Step IV.C (e.g., high confidence single nucleotide variants (SNVs)).

### (i) Step IV.C1

Step IV.C1 may include deploying three (or other numbers) independent substitution callers on raw data. Each independent substitution caller can be executed post processing to retrieve high-confidence passed Variant Call Files (VCF) and/or other types of files. The independent substitution callers may include CaVEMan (v1.7.5; https://github.com/cancerit/cgpCaVEManWrapper), MuTect2 (gatk:v4.0.1.2; https://github.com/broadinstitute/gatk), and/or Strelka2 (v2.9.1 with manta v1.3.1; https://github.com/lllumina/strelka).

### (ii) Step IV.C2

Step IV.C2 may include merging the passed calls according to (or based on) chromosome, position, REF and/or ALT alleles. A flagging step of one of the independent substitution callers (e.g., CaVEMan (cgpCavemanPostprocessing, v1.5.2)) can be applied to the merged calls. The variants may be annotated using one or more approaches, such as VEP (v92, https://github.com/Ensembl/ensembl-vep), VAGrENT (v3.3.0, https://github.com/cancerit/VAGrENT), gnomAD, COSMIC, and/or PON. In some embodiments, CaVEMan may consider a local purity and/or a copy number segmentation to optimize the detection of subclonal gene mutations in regions with aberrant copy number segmentation.

### (iii) Step IV.C3

Step IV.C3 may include filtering the merged calls to generate a list of high-confident substitutions. Said filtering may be performed according to one or more criteria. The one or more criteria may include that the called variants must pass all filters by at least 2 callers. The one or more criteria may include that the variants must not be present in the normal control file. The one or more criteria may include that the variants must not represent a common population polymorphism.

### (iv) Step IV.C4

Step IV.C4 may include rescuing hotspot mutations. In one example, a rescue process can be implemented to retrieve hotspot mutations in regions that are difficult to map. The hotspot mutations may satisfy one or more criteria. The one or more criteria may include an exact mutation match with an OncoKb hotspot mutation (or other mutations). The one or more criteria may include passing all filters in at least one variant caller. The one or more criteria may include being absent in the panel of normal controls. The one or more criteria may include a sufficient local depth (e.g., at least 20 reads or at least 25 reads) and/or support from at least 5 bidirectional reads (e.g., forward and reverse).

### (v) Step IV.C5

Step IV.C5 may include performing a variant prioritization. Said variant prioritization can be determined based on the level of evidence in the literature and/or public databases, and/or can include one or more criteria. The one or more criteria may include that a particular variant is annotated as oncogenic by the OncoKb database (or other databases). The one or more criteria may include that a variant class (e.g., truncation) is predicted as oncogenic by OncoKb (or other databases). The one or more criteria may include that the variant targets a tumor-suppressor gene (TSG) and/or oncogene, and is recurrent (e.g., at least 2 entries) in the COSMIC database (or other databases). The one or more criteria may include a prioritization by functional consequence of a mutation (e.g., hotspot). The one or more criteria may include that variants are sorted by decreasing VAF, wherein mutations present in the majority of the cells in the tumor (e.g., high VAF) are prioritized.

### (vi) Step IV.C6

Step IV.C6 may include reporting and/or providing high confidence mutations. The high confidence mutations can be reported in descending order of level of evidence (e.g., oncogenic > likely oncogenic > predicted oncogenic). The reporting may be confined to somatically acquired variants with aberrant read representation of at least 5% (or other percentage values that could be as low as the error rate of the sequencing technology used), and/or evidence in both forward and reverse NGS reads (e.g., bi-directional representation). For recurrent hotspot mutations, reporting thresholds can be limited to 2% (or other percentage values) variant allele reads with bi-directional representation. An integration with Cancer Cell Fraction (CCF) and/or local copy number estimates from Step III may enable reporting of the precise number of mutated alleles in a tumor (e.g., if a copy number is 5 and 2 alleles are mutated). An integration of gene mutations and gene expression may provide and/or specify further evidence on the functional consequences of the mutation (e.g., loss of function and/or down regulation of gene expression, activating mutations and over expression).

### (vii) Step IV.C7

Step IV.C7 may include performing a detailed mutation signature analysis (e.g., using as a reference the Alexandrov et al Nature 2020 mutation signatures and/or other signatures) to compute (e.g., using a signature fitting package such as Mutational Patterns or SigProfiler) the presence of clinically relevant mutation signatures associated with mutations in DNA repair genes (e.g., PMS2, MUTYH, and/or BRCA1/2).

### (viii) Application Embodiment 1

One example embodiment may include integrating SNV mutation signatures into putative germline findings, to further assess and/or validate pathogenicity of germline events (e.g., PMS2, MUTYH). Case study 7 illustrates such an example, in which a mutational burden and patterns gleaned from a cWGTS analysis resulted in attainment of a germline consent that subsequently identified a pathogenic PMS2 mutation.

### (ix) Application Embodiment 2

Certain types of tumors may have distinctive mutational signatures (e.g., known sets of specific mutational signatures, which can be used to rule out a differential diagnosis (e.g., Case Study 1 and Case Study 8). For example, in Case Study 1, the subject has an indeterminate diagnosis of neuroblastoma metastasis. However, a WGS analysis identified a pathognomonic schwannoma fusion gene, suggesting a different diagnosis. A tSNE analysis of RNA shows that the fusion gene clusters with another schwannoma, instead of neuroblastomas in the extended cohort, thereby confirming a schwannoma diagnosis. Moreover, a mutational signature analysis failed to identify the mutational signature seen in all neuroblastomas, ruling out the possibility of a neuroblastoma. In Case Study 8, for example, a differential diagnosis included metastatic retinoblastoma or neuroblastoma. However, the mutational pattern of the tumor precluded the possibility of neuroblastoma.

### Step IV.D: Detection of Somatic Insertions and Deletions

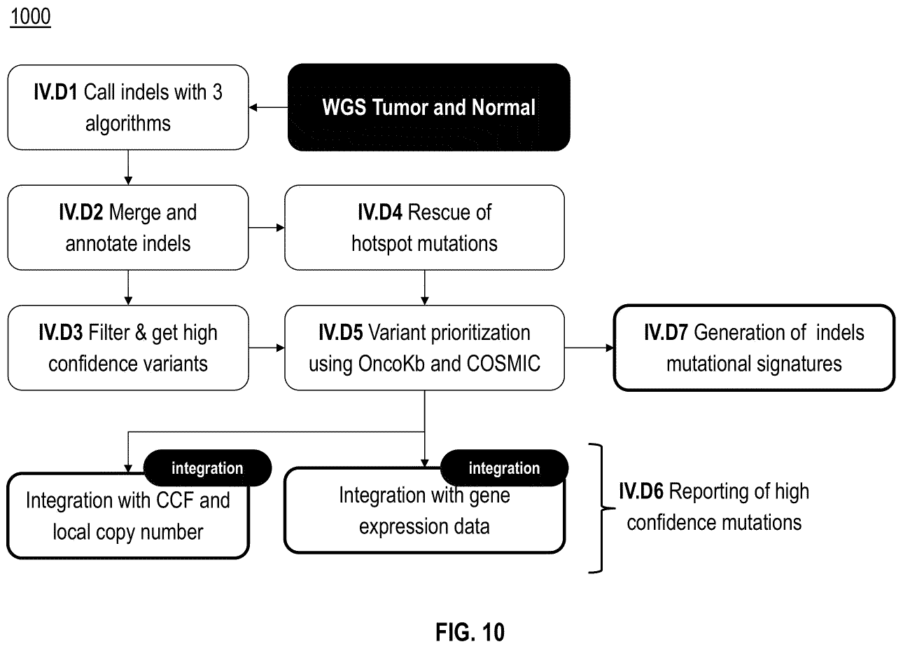

One or more inputs to Step IV.D can include an aligned matched tumor and/or normal BAM files (or other types of files). In some embodiments, the one or more inputs can include a BED file (or other types of files) of known cancer genes and hotspot mutations from OncoKB (or other databases). In some embodiments, the one or more inputs can include insertion and deletion calls in a reference panel of >98 normal tissue controls (or other numbers of normal tissue controls) analyzed by the analytics workflow. FIG. 10 depicts a diagram of an example process as described in Step IV.D (e.g., high confidence single nucleotide variants (SNVs)).

### (i) Step IV.D1

Step IV.D1 may include deploying, for example, at least two, such as three (or other numbers) independent variant callers on raw data. Each independent variant caller can be executed post processing to retrieve high-confidence passed Variant Call Files (VCF) and/or other types of files. The independent variant callers may include Pindel (cgpPindel v1.5.4; https://github.com/cancerit/cgpPindel), MuTect2 (gatk:v4.0.1.2; https://github.com/broadinstitute/gatk) and/or Strelka2 (v2.9.1 with manta v1.3.1; https://github.com/lllumina/strelka).

### (ii) Step IV.D2

Step IV.D2 may include normalizing (e.g., based on parsimony and left-alignment) and/or merging the passed calls according to (or based on) chromosome, position, REF and/or ALT alleles. The variants may be annotated using one or more approaches, such as VEP (v92, https://github.com/Ensembl/ensembl-vep), VAGrENT (v3.3.0, https://github.com/cancerit/VAGrENT), gnomAD, COSMIC, and/or PON.

### (iii) Step IV.D3

Step IV.D3 may include filtering the merged calls to generate a list of high-confident variant sets. Said filtering may be performed according to one or more criteria. The one or more criteria may include that the called variants must pass all quality control filters by at least 2 callers. The one or more criteria may include a Local Context Complexity (LCC) score > 0.5 for 10 bp (or some other score in some other number of bps) in 3′ and 5′ to minimize artifacts in regions of homopolymers (e.g., multiple stretches of the same base AAAAAAAAAAA). The one or more criteria may include that the variant is not detected in a panel of normal controls, analyzed by the analytics workflow. The one or more criteria may include that the variant does not represent polymorphisms in reference databases (1000 genomes).

### (iv) Step IV.D4

Step IV.D4 may include rescuing hotspot mutations. In one example, a rescue process can be implemented to retrieve hotspot mutations in regions that are difficult to map. The hotspot mutations may satisfy one or more criteria. The one or more criteria may include an exact mutation match with an OncoKb hotspot mutation (or other mutations). The one or more criteria may include passing all filters in at least one variant caller. The one or more criteria may include being absent in the panel of normal controls. The one or more criteria may include a sufficient local depth (e.g., at least 20 reads or at least 25 reads) and/or support from at least 5 bidirectional reads (e.g., forward and reverse).

### (v) Step IV.D5

Step IV.D5 may include performing a variant prioritization (e.g., hierarchy based on any number of ranked criteria). Said variant prioritization can be determined based on the level of evidence in the literature and/or public databases, and/or can include one or more criteria. The one or more criteria may include that a particular variant is annotated as oncogenic by the OncoKb database (or other databases). The one or more criteria may include that a variant class (e.g., truncation) is predicted as oncogenic by OncoKb (or other databases). The one or more criteria may include that the variant targets a tumor-suppressor gene (TSG) and/or oncogene, and is recurrent (e.g., at least 2 entries) in the COSMIC database (or other databases). The one or more criteria may include a prioritization by functional consequence of a mutation (e.g., hotspot).

### (vi) Step IV.D6

Step IV.D6 may include reporting and/or providing high confidence mutations. The high confidence mutations can be reported in descending order of level of evidence (e.g., oncogenic > likely oncogenic > predicted oncogenic). The reporting may be confined to somatically acquired variants with aberrant read representation of at least 5% (or other percentage values, which could be as low as the error rate of the sequencing technology used), and/or evidence in both forward and reverse NGS reads (e.g., bi-directional representation). For recurrent hotspot mutations, reporting thresholds can be limited to 2% (or other percentage values) variant allele reads with bi-directional representation. An integration with Cancer Cell Fraction (CCF) and/or local copy number estimates from Step III may enable reporting of the precise number of mutated alleles in a tumor (e.g., if a copy number is 5 and 2 alleles are mutated). An integration of gene mutations and gene expression may provide and/or specify further evidence on the functional consequences of the mutation (e.g., loss of function and/or down regulation of gene expression, activating mutations and over expression).

### (vii) Step IV.D7

In step IV.D7, indel signatures may be computed (e.g., the events are classified into categories) and/or used to determine if the event is a repeat-mediated deletion, microhomology associated, and/or insertion (e.g., helps better diagnose DNA repair deficiencies).

### (viii) Application Embodiment 1

One example embodiment may include identification of a rare disease defining indel. For example, in Case Study 9, a cWGTS analysis identified an oncogenic indel KBTBD4, a rare cancer gene not covered by a majority of panel based tests.

### vi. Step V: Assessment of Microsatellite Instability and Mutational Burden Across Variant Classes

One or more inputs to Step V can include a merged high-confidence set (e.g., genome-wide calls detected and passed all filters by two or more callers) of acquired RNA-fusions (e.g., Step IV.A). In certain embodiments, the one or more inputs may include a merged high-confidence set (e.g., genome-wide calls detected and passed all filters) of acquired structural variants (e.g., Step IV.B). In some embodiments, the one or more inputs may include a merged high-confidence set (e.g., genome wide calls detected and passed all filters) of acquired substitutions (e.g., Step IV.C). In some embodiments, the one or more inputs may include a merged high-confidence set (e.g., genome wide calls detected and passed all filters) of acquired insertions and deletions (e.g., Step IV.D).

### Step V.1

In Step V.1, certain approaches for assessing microsatellite instability, such as MSISensor, may be deployed/applied, and/or a derivative score (e.g., MSI Score) can be presented. The samples with an MSI Score of, for example, > 10 (or other values) can be classified as MSI High, while samples with an MSI Score, for example, between 5 and 10 (or other values) may be classified as MSI Medium. In certain embodiments, samples with an MSI Score, for example, < 5 may be classified as MSI Low.

### Step V.2

Step V.2 can include calculating a tumor burden. The tumor burden may be calculated using (or according to) one or more criteria/considerations. The one or more criteria may include that complex structural variants are collapsed into unique structural variant clusters to avoid over estimation of structural variant burden. The one or more criteria may include that substitutions, insertions and/or deletions are merged into one file representing small mutations. The one or more criteria may include that for each variant class (e.g., structural variants, small mutations and/or fusion genes), a mutation burden per Megabase of the human genome can be reported for mutations targeting coding regions of the genome, as well as genome-wide mutation patterns.

### vii. Step VI: Detection of Germline Variants

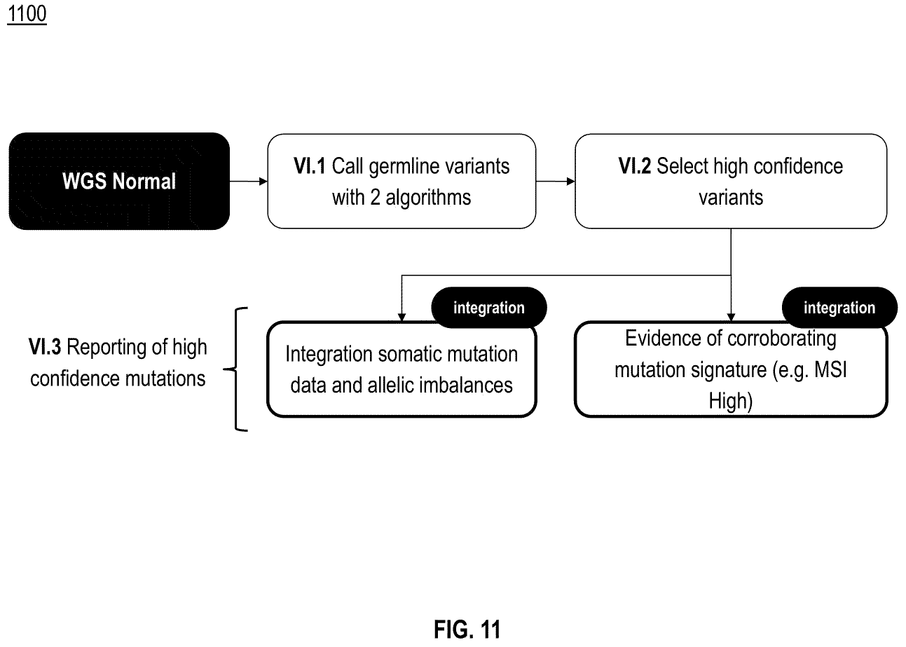

One or more inputs to Step VI can include an aligned bam file from normal sample(s). In certain embodiments, the one or more inputs can include a reference set of calls from 98 normal subjects (or other numbers of subjects). The analytics workflow can be applied to said reference set of calls to filter out platform-specific artifacts and/or common SNPs missed by public databases. In some embodiments, the one or more inputs may include a reference input of known germline mutations associated with cancer (e.g., Appendix 5). FIG. 11 depicts a diagram of an example process as described in Step VI (e.g., germline analysis)

### Step VI.1

Step VI.1 may include performing a germline variant detection. In some embodiments, at least two independent germline callers can be deployed on (or applied to) data. The at least two independent germline callers can include Strelka2 germline and FreeBayes (v1.2.0; https://github.com/ekg/freebayes). The variants may be annotated using one or more approaches, such as VEP (v92, https://github.com/Ensembl/ensembl-vep), VAGrENT (v3.3.0, https://github.com/cancerit/VAGrENT), gnomAD, and/or COSMIC.

### Step VI.2

Step VI.2 may include selecting high-confidence germline variants. The high-confidence germline variants can be selected according to (or based on) one or more criteria. The one or more criteria may include that the variants are detected by, and passed, all filters in both callers. The one or more criteria may include that the variants must occur within the gene body of established cancer predisposition genes (e.g., Appendix 5). The one or more criteria may include that the variants must have a population frequency of less than <5% (or other percentage values) in Gnomad databases (or other databases). The one or more criteria may include that the variants must be absent from the panel of healthy controls analyzed by the analytics workflow. The one or more criteria may include that the mutation cannot be annotated as benign or likely benign by any source in CLINVAR (or other databases/archives).

### Step VI.3

Step VI.3 may include a reporting of germline mutations. Population filtering, database comparison, and/or somatic data integration can be performed using methods in accordance with the American College of Medical Genetics and ClinGen Somatic/Germline Data Integration subcommittee. In some embodiments, variants can be prioritized using one or more criteria. The one or more criteria may include annotation categories of CLINVAR (e.g., in descending priority of pathogenic labels) and/or a frequency in Gnomad (e.g., lower population frequency ranks higher). In certain embodiments, the one or more criteria may include evidence of a second acquired mutation in a same gene in the somatic mutation data (e.g., Step IV) and/or allelic imbalances (e.g., deletion or loss of heterozygosity). In certain embodiments, the one or more criteria may include evidence of a corroborating mutation signature (e.g., MSI High status in individuals with mutations targeting mismatch repair genes).

### Application Embodiment 1

In certain embodiments, germline variants of unknown significance (VUS) can be assessed in conjunction with somatic genome wide signatures (e.g., MUTYH and/or PMS2). For example, in Case Study 7, a mutational burden and/or patterns gleaned from cWGTS analysis resulted in attainment of a germline consent, which subsequently identified a pathogenic PMS2 mutation.

### Application Embodiment 2

In certain embodiments, the WGS germline pipeline may report and/or specify germline events in rare hereditary genes that may not be sequenced by targeted panels (e.g., BARD1, SBDS, EP300, and/or EXT2). In some embodiments, the WGS germline pipeline can be used to assess non-coding events. Case study 10 illustrates such an example, in which there is a pathogenic germline mutation in BARD1.

### viii. Step VII: Clonality Analysis

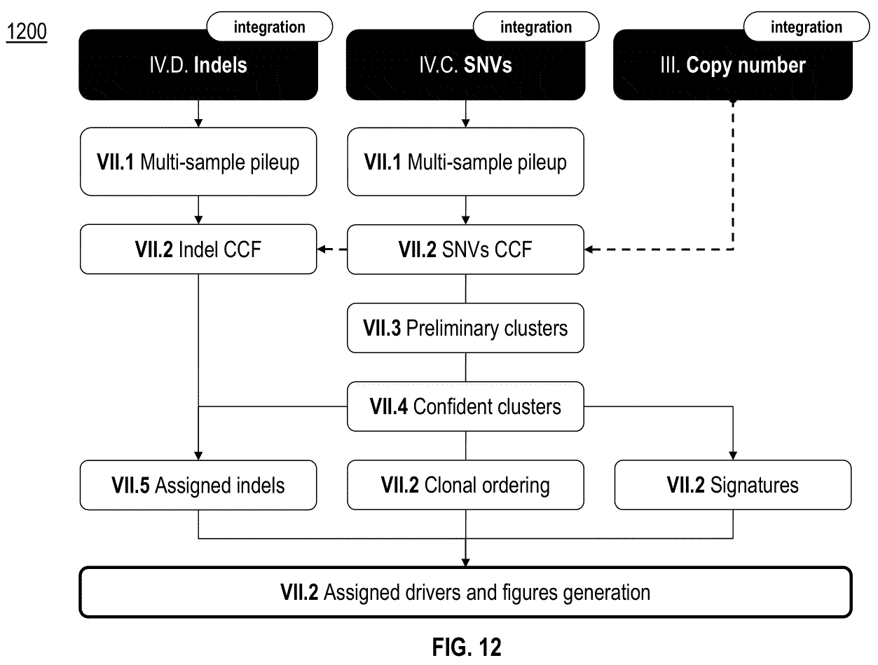

One or more inputs to Step VII can include or correspond to data and/or information from one or more samples of a same subject. For instance, the one or more inputs may include a confident set of SNVs (e.g., Step IV.C), a confident set of indels (e.g., Step IV.D), and/or purity and copy number segments (e.g., Step III). FIG. 12 depicts a diagram of an example process as described in Step VII (e.g., clonality analysis).

### Step VII.1

In embodiments with multiple biopsies, the union of the variants (e.g., confident SNVs/indels) can be piled-up and/or aggregated for support in each individual sample.

### Step VII.2

In step VII.2, the purity and local copy numbers can be used to scale the VAF of the SNVs and/or indels to cancer cell fraction (CCF).

### Step VII.3

Step VII.3 may include clustering, using one or more Dirichlet Processes (or other suitable stochastic processes), the CCF values of the SNVs. The SNVs and the indels may be assigned to the generated clusters.

### Step VII.4

In certain embodiments, clusters associated with artifacts (e.g., artifactual clusters) can be removed, according to one or more criteria (e.g., in a predetermined sequence, such as from 4.1 to 4.4 – it is noted that order may vary, except 4.4 which is not a filter criterion but the final step for returning the confident clones). The one or more criteria may include:
4.1: A cluster can have at least 50 (or other values) assigned mutations (e.g., for a single sample case; in multiple biopsies the number of mutations decreases), or 5% of total mutations.
4.2: Clusters with more than 40% (or other percentage values), such as 60%, of mutations from a single chromosome can be removed (e.g., cluster is likely due to a missed copy number segment).
4.3: Clusters that have a low prevalence (e.g., 0 < prevalence < 20% in multiple samples) in a plurality of samples, without being highly represented in any single sample, can be removed.
4.4: If less than two clusters remain, the process can end, and/or no results are returned (e.g., the sample does not have multiple clones to assess). Clusters can be sorted and/or renamed by prevalence across the samples. As such, cluster 1, for example, can be the trunk (e.g., fully clonal across all samples).

### Step VII.5

In Step VII.5, the indels may be assigned the closest resulting confident clones according to the mean CCF.

### Step VII.6

In Step VII.6, clonal ordering can be deduced by using certain approaches, such as clonevol (https://github.com/hdng/clonevol). The clonal ordering can include slightly adjusting the clone prevalences in descending order, to ensure that the Pigeonhole principle is not violated.

### Step VII.7

In Step VII.7, SNV mutational signatures may be processed for the mutations in each clone individually (e.g., as described in Step IV.C).

### Step VII.8

In Step VII.8, a plurality of figures, showing the clonal representation across the samples, can be generated. Within the report, the predicted SNV and indel drivers may be drawn on the clonality tree.

### Application Embodiment 1

In certain embodiments, knowledge of a clonal structure can be important for selecting targeted treatments. For example, a clinician may select a treatment that targets one or more mutations shared across the samples of the subject.

### Application Embodiment 2

Clonality analysis may be helpful in applications associated with disease monitoring. For instance, clonality analysis can provide insight into the evolution of a subject’s cancer, and can detect the emergence of treatment resistant subclones within the tumors. The presence of chemotherapy-associated mutational signatures in a subclone, for instance, may be associated with resistance to said treatment.

### ix. Step VIII: SVs and Fusions Burden

One or more inputs to Step VIII can include or correspond to high-confidence structural variants (SV) from tumor-derived DNA and/or high-confidence fusions from tumor derived RNA. In some embodiments, the one or more inputs may include proprietary cohort-level SV data derived from the tumors in the cohort as a reference. In certain embodiments, the one or more inputs may include proprietary cohort-level fusion data derived from the tumors in the cohort as a reference.

### Step VIII.1

In some embodiments, Step VIII.1 may include comparing a number of SVs and gene fusions of the current tumor with the entire cohort taking.

### Application Example 1

In certain embodiments, a majority of fusions identified in cancer may result in out-of-frame transcripts that are capable of producing long novel protein sequences and neoantigens, wherein the long novel protein sequences and neoantigens can elicit antitumor immune responses. A simultaneous analysis of SV and fusion gene burden can aid in prioritizing tumors that may benefit from an immune checkpoint inhibition therapy (e.g., Case Study 11 and Case Study 12) and/or other therapies. The subjects of Case Study 11 and Case Study 12, for example, have a high SV and/or fusion gene burden compared to the rest of the cohort. The subjects of Case Study 11 and Case Study 12 received immune checkpoint blockade therapy, and achieved a complete response (e.g., remaining disease-free).

### x. Step IX: Reporting of Summary Findings

Upon performing variant detection, annotation and/or filtering, high priority variants (e.g., Steps III, IV, V, VI) of diagnostic relevance can be automatically embedded and/or included in a report. The report may provide a summary of the analytics workflow findings, where the report can be prototyped according to established reports derived from panel testing. In certain embodiments, the report may include and/or provide one or more of the following:
One or more somatic mutations in established cancer genes (e.g., small mutations, structural variants, copy number alterations, and/or fusion genes).
One or more somatic mutations of relevance in regulatory regions (e.g., small mutations and/or structural variants).
One or more germline mutations in established cancer predisposition genes.
A tumor mutation burden across variant classes (e.g., small mutations, structural variation, and/or fusion genes)
A microsatellite instability score and status (e.g., low, intermediate, and/or high)
One or more OncoKB annotations (e.g., a level of evidence)
A gene expression of the mutated genes.

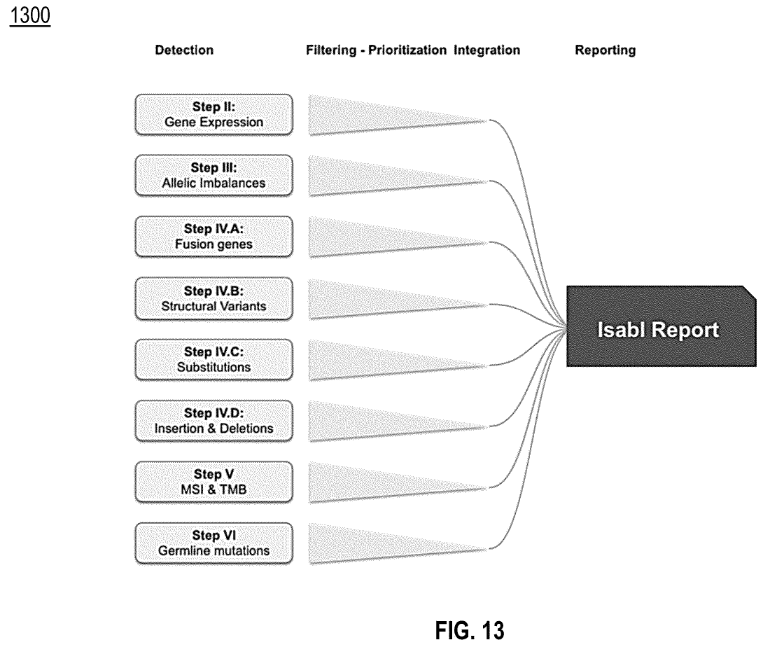

FIG. 13 depicts a diagram of an example process as described in Step IX (e.g., summary reporting).

### xi. Case Studies

### Case Study 1

A subject (e.g., IID_H135066) is a male diagnosed at an age of 5 years with stage IV neuroblastoma, metastatic to the bone and bone marrow. The subject is initially refractory to treatment, and as such, receives investigational treatment with good response. However, the subject experiences metastatic relapse in the right frontal bone, wherein the right frontal bone is surgically removed. After removal of the right frontal bone, the subject remains disease-free for approximately 2 years.

After a certain period, the subject undergoes excisional biopsy due to a slowly increasing right occipital lobe lesion. The tissue extracted from the biopsy is analyzed using a WGS analysis. Given the subject’s history of cancer, it is not clear if the current tumor (e.g., tumor in the right occipital lobe) collected 10 years after the initial neuroblastoma diagnosis is a relapse of the neuroblastoma or a new type of cancer.

The WGS analysis shows that the current tumor lacks a mutational signature seen in neuroblastomas. Moreover, the WGS analysis identifies a SH3PXD2A-HTRA1 fusion. In certain embodiments, the SH3PXD2A-HTRA1 fusion can be a pathognomonic event for schwannoma. The RNAseq detects the same fusion product, supporting the diagnosis of schwannoma. Indeed, a tSNE clustering of RNAseq data shows that the tumor is closest to another case of schwannoma with the same fusion.

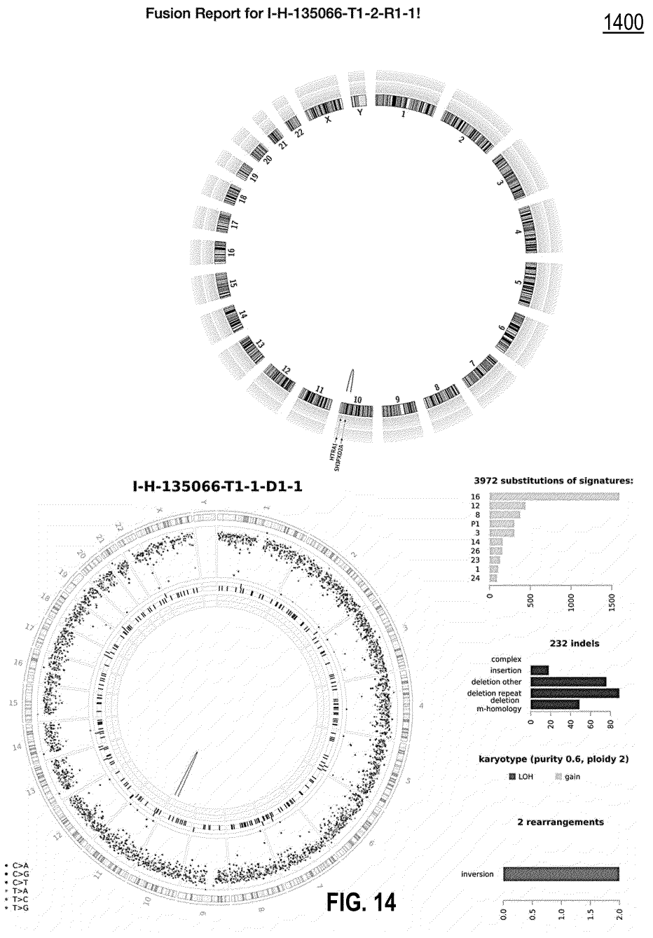

FIG. 14 depicts the genomic findings for the subject of Case Study 1 (e.g., IID_H135066). The upper portion of FIG. 14 illustrates a Circos plot. The Circos plot can illustrate a pathognomonic fusion, thereby clarifying the diagnosis of schwannoma. The lower portion of FIG. 14 illustrates a Circos plot and a signature analysis of the different variant classes. There is no evidence to suggest the tumor is associated with a neuroblastoma signature (e.g., signature-18).

### Case Study 2

A subject (e.g., IID_201628) is a female diagnosed at an age of 11 years with schwannoma. A cWGTS analysis identifies a difficult-to-interpret and complex genomic rearrangement on chromosome-10. A RNAseq analysis shows that this complex event creates a pathognomonic schwannoma fusion (e.g., SH3PXD2A-HTRA1). Moreover, the subject’s tumor clusters with another schwannoma tumor where the same fusion is created by a simple deletion event, thereby confirming the oncogenic nature of the complex genomic event.

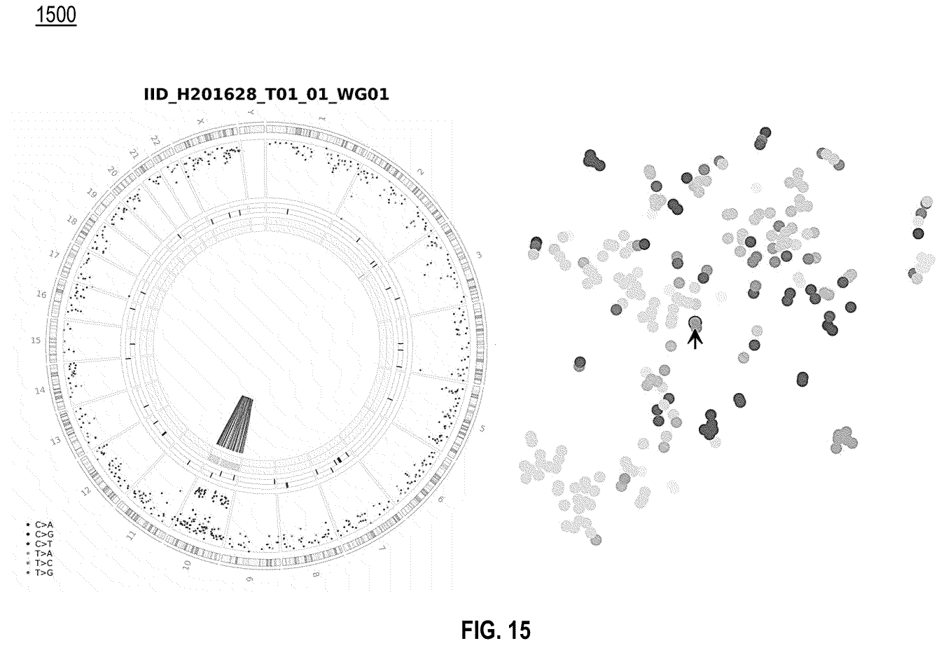

FIG. 15 depicts the genomic findings for the subject of Case Study 2 (e.g., IID_201628). The left portion of FIG. 15 illustrates a Circos plot. Said Circos plot illustrates the complex genomic rearrangement of chromosome 10. The right portion of FIG. 15 illustrates a tSNE map. The tSNE map illustrates that the subject of Case Study 2 (e.g., IID_H201628, indicated by an arrow) is closest to another schwannoma (e.g., associated with the subject of Case Study 1, IID_H 135066).

### Case Study 3

A subject (e.g., H134768) is diagnosed with embryonal rhabdomyosarcoma in the absence of the cardinal alveolar rhabdomyosarcoma fusions (e.g., PAX3-FOXO1 and PAX7-FOXO1). A cWGTS analysis identifies a complex genomic rearrangement resulting in a translocation between PAX3 and a new partner FOXO3. A tSNE clustering indicates that the tumor is closest to other alveolar rhabdomyosarcoma cases with canonical fusions (e.g., PAX3-FOXO3 and PAX7-FOXO3), thereby confirming the diagnosis.

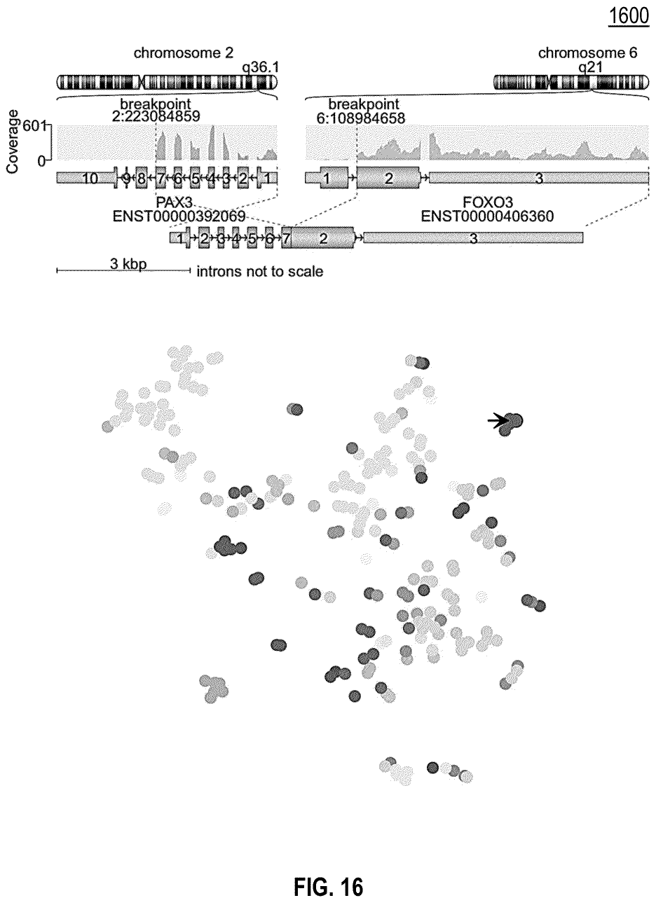

FIG. 16 depicts the genomic findings for the subject of Case Study 3 (e.g., H134768). The upper portion of FIG. 16 illustrates the unreported new fusion product for the PAX3 gene (PAX3-FOXO3). The lower portion of FIG. 16 illustrates a tSNE map. The tSNE map illustrates that the subject of Case Study 3 (e.g., H134768, indicated by an arrow) is closest to other alveolar rhabdomyosarcomas.

### Case Study 4

A subject (e.g., IID_H201633) is a female diagnosed with a desmoid-type fibromatosis in the back. The desmoid-type fibromatosis is a benign growth caused by mutations in the WNT/APC/Beta-catenin pathway. In rare cases, however, the disease is aggressive and cancerous. In Case Study 4, the subject has a local recurrence five years after the removal of the primary tumor, and a multifocal recurrence 7.5 years from diagnosis. A cWGTS analysis fails to detect alterations in the Beta-catenin pathway. However, the cWGTS analysis identifies a complex genomic rearrangement involving five chromosomes, resulting in an over-expression of the TERT gene. Although not-yet targetable, the complex genomic rearrangement can explain the aggressive behavior of the disease.

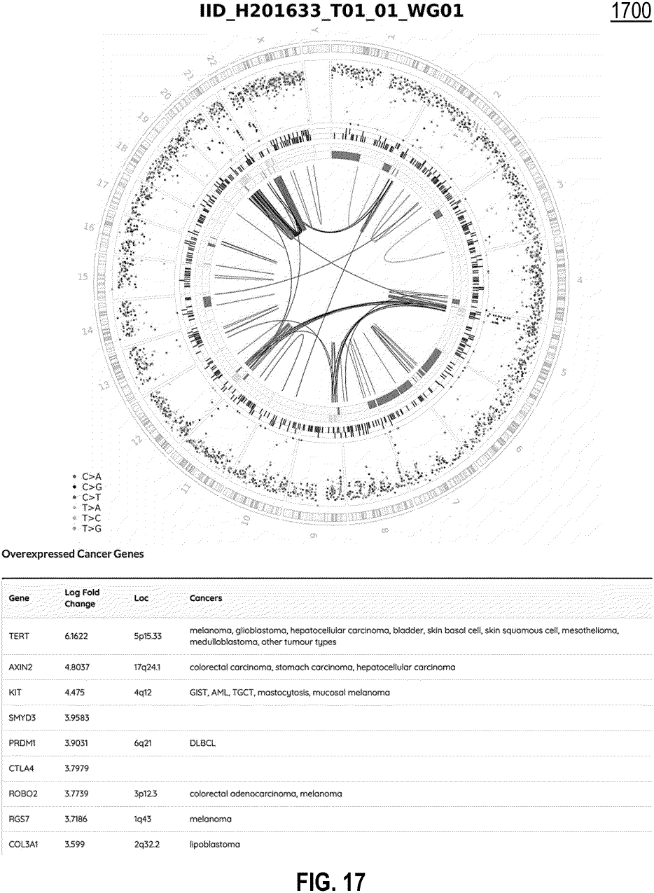

FIG. 17 depicts the genomic findings for the subject of Case Study 4 (e.g., IID_H201633). The upper portion of FIG. 17 illustrates a Circos plot. Said Circos plot illustrates a massive chromothripsis event. The chromothripsis event involves chromosomes 5, 9, 12, 20 and 22, affecting the TERT locus on chromosome 5p. Said complex leads to an over-expression of the TERT gene, as shown in the lower portion of FIG. 17.

### Case Study 5

A subject (e.g., H135393) is diagnosed with a small blue round cell sarcoma. A WGTS analysis identifies a BCOR-CCNB3 fusion. The BCOR-CCNB3 fusion defines a subset of this disease entity associated with better prognosis compared to other subtypes (such as CIC-DUX4 [29300189]).

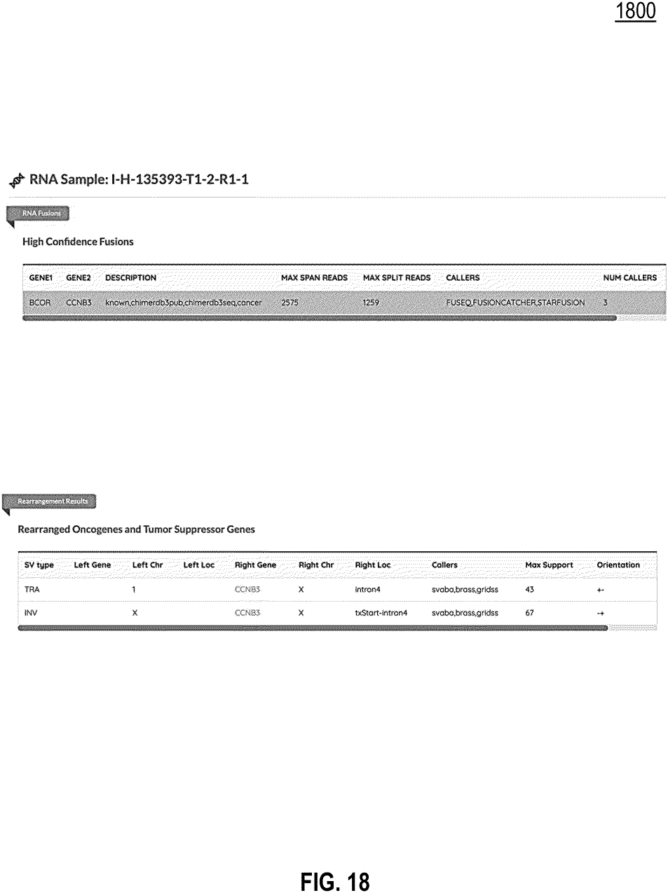

FIG. 18 depicts the diagnostic and/or prognostic genomic findings for the subject of Case Study 5 (e.g., H135393). The upper and lower portions of FIG. 18 illustrate the fusion genes as identified from the RNAseq analysis and the WGS analysis.

### Case Study 6

A subject (e.g., H133676) is diagnosed with adenoid cystic carcinoma. A standard clinical assessment is unable to provide information of the oncogenic events affecting MYB and NFIB (e.g., known drivers of this disease entity). A WGS analysis identifies a silent genome with only four genomic rearrangements. The silent genome is involved in a chain of complex structural variants amongst chromosomes 6, 9 and 18. The associated breakpoints for the genomic event fall at 4 Kb from MYB TSS, and in an enhancer region at 10 Kb from NFIB. Although the RNAseq analysis fails to detect any fusions between the two genes, the analysis shows that the tumor is associated with an outlier gene expression of MYB. Said findings suggest that the overexpression is caused by through “hijacking” of an NFIB enhancer, thereby clarifying the oncogenic nature of this event.

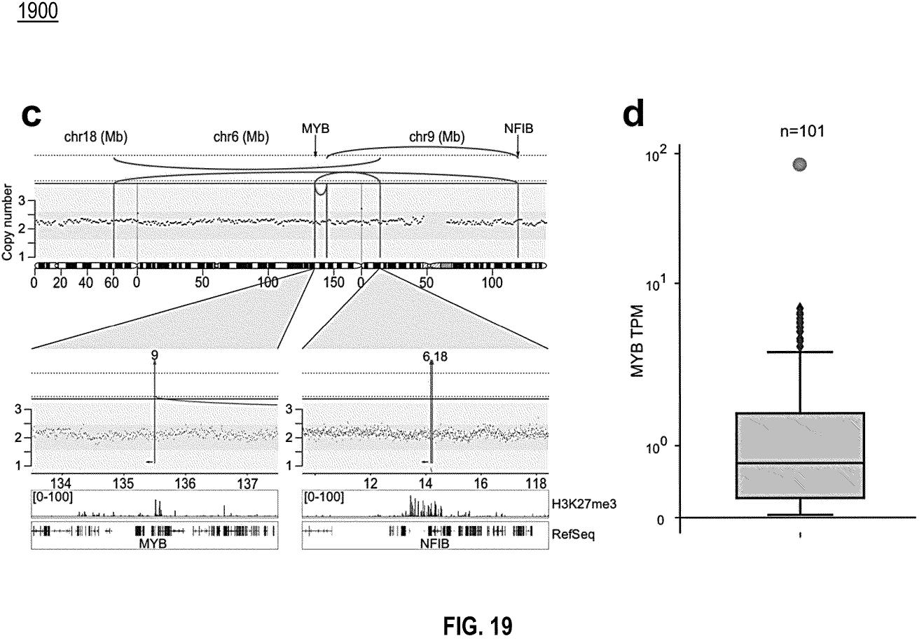

FIG. 19 depicts the genomic findings for the subject of Case Study 6 (e.g., H133676). The left portion of FIG. 19 illustrates the genomic rearrangements that bring the enhancers from NFIB to MYB. The H3K27me3 marks in the bottom panels of FIG. 19 indicate and/or specify the enhancers. The right portion of FIG. 19 illustrates the expression (e.g., in TPM) of MYB across the cohort, wherein the subject of Case Study 6 is associated with the outlier MYB expression (e.g., the dot).

### Case Study 7

A subject (e.g., 201472) is diagnosed at an age of 12 years with osteosarcoma. A cWGTS analysis identifies a hypermutation across variant classes (e.g., TMB=16.7, Indel=89,588, SNVs=31,520, SVs=568) with an enrichment for repeat-mediated deletions, consistent with a microsatellite instable genome. Said observations prompt an attainment of consent to perform germline testing. The germline testing results in identification of a PMS2 mutation (e.g., p.D699H) annotated as a likely pathogenic/VUS, and a somatic loss of the wild type allele. Said results highlight how composite readouts from a cWGTS analysis (e.g., a germline mutation, an allele specific copy number, and/or a genome-wide TMB) can provide corroborating evidence for the functional assessment of predisposed germline mutations.

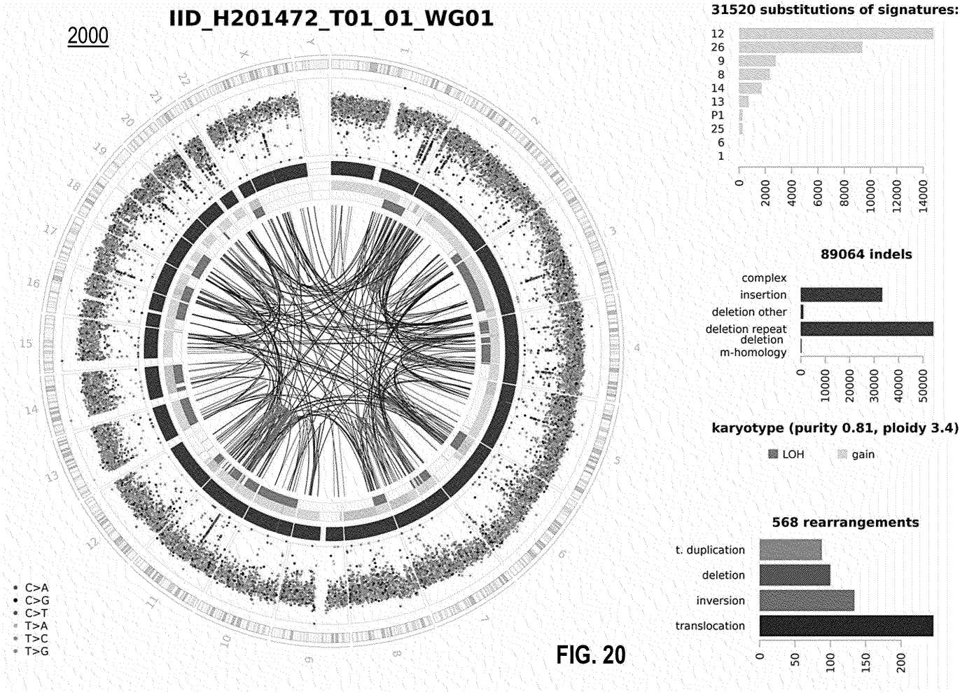

FIG. 20 depicts a genome-wide distribution and patterns of somatic mutations for the subject of Case Study 7 (e.g., H201472). The left portion of FIG. 20 illustrates a Circos plot. The Circos plot illustrates different types of somatic mutations along the genome. The outermost ring of the Circos plot depicts the intermutation distance for the SNVs (e.g., coded by the pyrimidine partner of the mutated base). The middle ring of the Circos plot depicts small insertions and deletions. The innermost ring of the Circos plot depicts the changes of a copy number, wherein the arcs illustrate the SVs. The middle panel of FIG. 20 is a bar plot illustrating the absolute number of mutations attributed to the ten mutational signatures with the highest exposure in the tumor. The right panel of FIG. 20 illustrates the mutation signature analysis for substitution, indels and rearrangements

### Case Study 8

A subject (e.g., H133671) is diagnosed at an age 2.5 years with retinoblastoma. Less than 2 years later, the subject presents a large abdominal mass. Tumor pathology suggests an undifferentiated small blue round cell tumor with neuroendocrine differentiation. A differential diagnosis includes a systemic metastasis of retinoblastoma (e.g. very rare) or a neuroblastoma (e.g., frequently presents as an abdominal mass). A WGTS analysis shows that the abdominal tumor has a high-level amplification of the MYCN gene. Moreover, the abdominal tumor clusters with MYCN-amplified neuroblastomas in the absence of any other retinoblastoma in the extended cohort. However, a mutational signature analysis suggests that the tumor lacks a signature-18 seen in neuroblastomas (e.g., FIG. 18). The results of the mutational signature analysis rule out the neuroblastoma differential diagnosis.

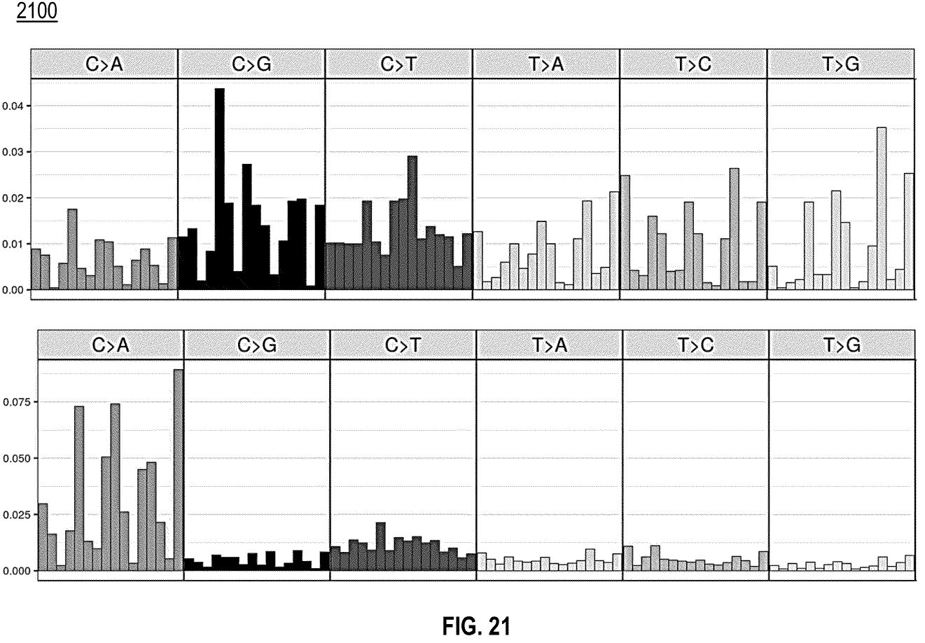

FIG. 21 depicts mutational profiles of the subject of Case Study 8 (e.g., H133671). The top portion of FIG. 21 illustrates the mutational profiles of the subject (e.g., subject with an indeterminate diagnosis). The lower portion of FIG. 21 illustrates the mutational profiles of the closest neighbor in tSNE. The closest neighbor in tSNE is a neuroblastoma tumor (e.g., H136649). There is no preponderance of C>A mutations (first panel from left to right) in the retinoblastoma tumor.

### Case Study 9

A subject (e.g., H197215) is diagnosed with a pineal parenchymal tumor. A cWGTS analysis identifies an indel in KBTBD4 (p.R313_M314insPRR). Said indel is a disease defining mutation in a rare cancer gene not usually covered by targeted gene sequencing panels.

### Case Study 10

A subject (e.g., H156418) is diagnosed with neuroblastoma. A cWGTS analysis identifies a pathogenic mutation in BARD1 (p.E652fs*69).

### Case Studies 11 and 12

In Case Study 11, a subject (e.g., IID_H135022) is diagnosed at an age of 13 years with metastatic adrenocortical carcinoma and on-treatment progression. A previous targeted sequencing assay of the tumor failed to indicate any informative biomarkers. A cWGTS analysis shows that the tumor has a profoundly rearranged genome, scoring the tumor of the patient as the highest in gene fusion burden and the second highest in structural variant burden within the pediatric tumor cohort. Said results raise the possibility of checkpoint blockade therapy for the subject. The subject receives nivolumab/ipilimumab, and shows a complete response after three cycles of therapy. The subject is free of disease 22 months after therapy cessation.

In Case Study 12, a subject (e.g., IID_H135462) is diagnosed at an age of 14 years with relapsed refractory clear cell carcinoma of the vagina, with progressive, on-treatment, metastatic disease. A cWGTS analysis indicates that the tumor has a high-gene fusion and SV burden. As such, the subject receives checkpoint blockade therapy. The subject shows a complete response to therapy in 6 months, remaining free of disease six months after therapy.

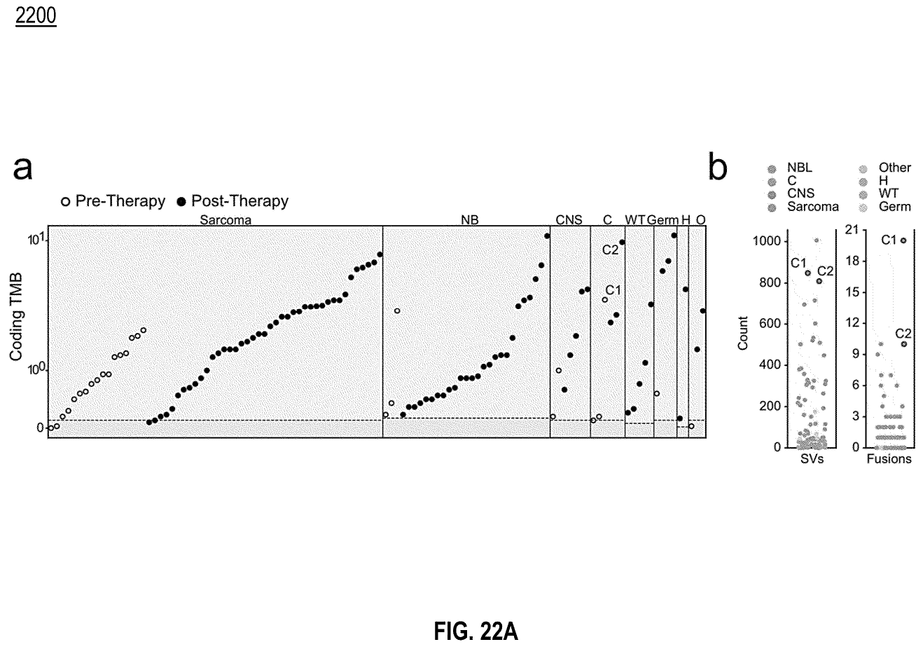

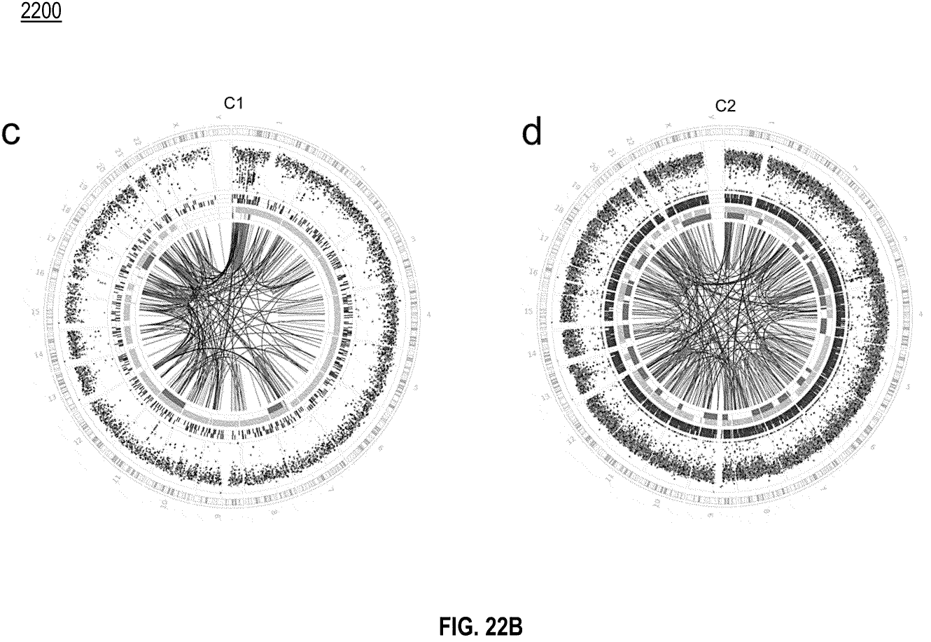

Panel (a) of FIG. 22A depicts a distribution of coding tumor mutational burden (TMB) as assessed by a WGS analysis across the cohort (n=114). The dotted line indicates a median coding TMB (e.g., SNVs and Indels) as previously reported by Grobner et al. The subjects are grouped by disease category (e.g., NB: Neuroblastoma, CNS: Central nervous system, C: Carcinoma, WT: Wilms tumor, Germ: Germ cell tumor, H: Hepatoblastoma, O: Other). The subjects with a carcinoma diagnosis (e.g., subject C1 (H135022) and subject C2 (H135462)) who responded to immunotherapy are labeled. Panel (b) of FIG. 22A depicts a distribution of structural variants (SV) (e.g., right portion of panel (b)) and gene fusion burden (e.g., left portion of panel (b)) across the samples with both WGS and RNAseq available (e.g., n=101). Subject C2 has a RNA sample with poor quality. As such, clonal fusions from another time point from subject C2 are shown. Panel (c) of FIG. 22B depicts a genome-wide distribution and patterns of somatic mutations for the tumor of subject C1 (e.g., H135022), a patient with metastatic adrenocortical carcinoma, depicting a high a SV burden. Panel (d) of FIG. 22B depicts a genome-wide distribution and patterns of somatic mutations for the tumor of subject C2 (H135462), a patient with metastatic adrenocortical carcinoma, depicting a high SV burden.

### Methods for Performing a cWGTS Analysis

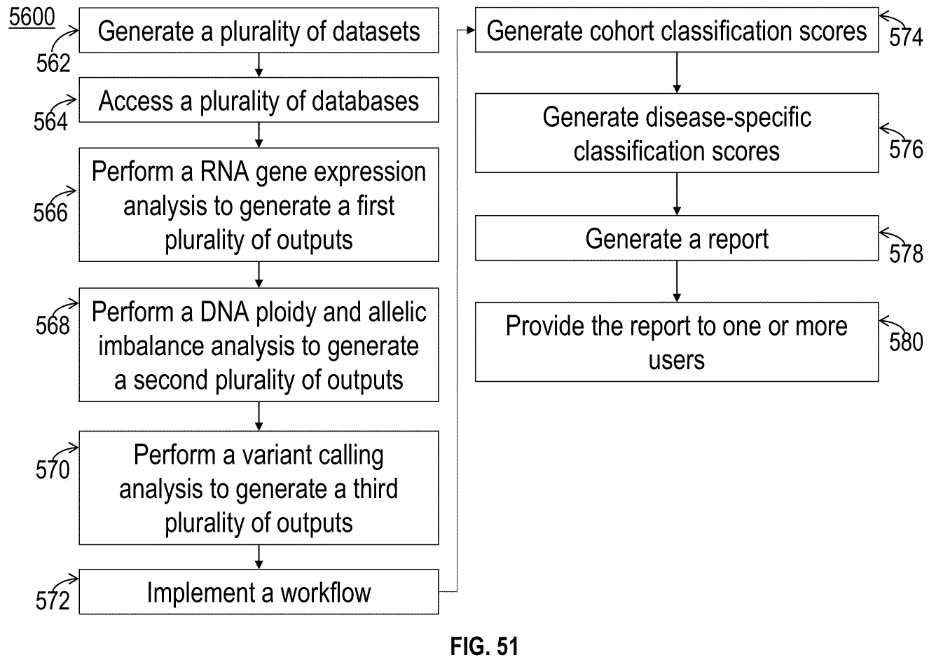

Referring to FIG. 51, depicted is a flow diagram of embodiments of a method for performing a cWGTS Analysis. The functionalities of the method may be implemented using, or performed by, the components detailed herein in connection with FIGS. 1 and 2. In some embodiments, process 5600 can be performed by a client computer system 114. In some embodiments, process 5600 can be performed by other entities, such as a server system 100 (as discussed in FIG. 1). In some embodiments, process 5600 may include more, fewer, or different steps than shown in FIG. 51.

In brief overview, process 5600 can include generating a plurality of datasets (562). The process 5600 may include accessing a plurality of databases (564). The process 5600 may include performing a RNA gene expression analysis to generate a first plurality of outputs (566). The process 5600 may include performing a DNA ploidy and allelic imbalance analysis to generate a second plurality of outputs (568). The process 5600 can include performing a variant calling analysis to generate a third plurality of outputs (570). The process 5600 can include implementing a workflow (572). The process 5600 can include generating cohort classification scores (574). The process 5600 can include generating disease-specific classification scores (576). The process 5600 can include generating a report (578). The process 5600 can include providing the report to one or more users (580).

Referring now to operation (562), and in some embodiments, a plurality of datasets can be generated. For example, the plurality of datasets (e.g., a first dataset, a second dataset, and/or other datasets) can be generated based on (or according to) sequencing of a tumor sample and/or a healthy control germline sample (e.g., from tissue, such as peripheral blood, nails, skin, and/or adjacent normal tissue). In certain embodiments, the tumor sample can be a clinical sample selected from the group consisting of fresh tissue, frozen tissue, formalin-fixed paraffin-embedded tissue (FFPE), blood, cfDNA, plasma, cerebral spinal fluid, and/or serum. The plurality of datasets may comprise a first dataset, a second dataset, a third dataset, and/or other datasets. The first dataset can be based on whole transcriptome sequencing of RNA in the tumor sample obtained from a patient. The second dataset may be based on a whole genome sequencing (WGS) of DNA derived from the tumor sample obtained from the patient. The third dataset can be based on WGS of DNA in the healthy control germline sample.

In some embodiments, the second and/or third datasets can have at least one of (1) a quality score (e.g., calculated according to Mosdepth), (2) a coverage metric, and/or (3) a tumor cell content (e.g., a tumor purity derived using a Battenberg approach). The quality score may satisfy a quality threshold. The coverage metric (e.g., a median coverage) may satisfy a coverage threshold (e.g., median coverage in the normal tissue of at least 30x, and/or a median coverage in the tumor sample of at least 60x). In certain embodiments, the tumor cell content may satisfy a tumor purity threshold. In one example, the quality score may indicate, provide, and/or specify a genome mapping quality. In some embodiments, the quality threshold can be at least 20 (or other values) Phred (e.g., to calculate a high quality depth for certain files, such as DNA bam files). In some embodiments, the coverage score may indicate and/or specify the genome coverage. The coverage threshold can be at least about 70% (or other percentage values) genome coverage. In some embodiments, the tumor cell content may indicate a tumor purity. The tumor purity can correspond to (or be associated with) the DNA in the tumor sample. In certain embodiments, the tumor purity threshold can be at least about 20% (or other percentage values) tumor purity.

Referring now to operation (564), and in some embodiments, a plurality of databases may be accessed and/or used. The plurality of databases may comprise a first reference database, a second reference database, a third reference database, and/or other databases. The first reference database (e.g., proprietary cohort level database, such as feather.db) may include, store, and/or maintain a plurality of individual sample gene expression transcripts per million (TPM) values (e.g., SALMON (or other approaches) can be used to calculate the TPM values). Said plurality of individual sample gene expression TPM values can be for a reference cohort of tumor samples. The second reference database may include, store, and/or maintain annotations for at least one of (i) RNA fusions, (ii) somatic structural variants, (iii) somatic substitutions, (iv) somatic insertions and deletions (indels), (v) microsatellite instability and/or mutational burden scores for each variant class, (vi) germline variants, (vii) somatic mutation patterns or signatures in each sample, and/or (vii) allelic imbalances. Said annotations can be for a reference cohort of tumor samples, at an individual sample level. The third reference database (e.g., OncoKb and/or other databases) may include, store, and/or maintain a plurality of gene identifiers. The plurality of gene identifiers may correspond to (or be associated with) a plurality of known cancer genes.

Referring now to operation (566), and in some embodiments, an RNA gene expression analysis can be performed. The RNA gene expression analysis can be performed using (or according to) the first dataset (and/or other datasets), the first reference database (e.g., feather.db), and/or the third database (e.g., OncoKb). The RNA gene expression analysis can be performed to generate and/or determine a first plurality of outputs for the tumor sample (e.g., list of priority aberrantly expressed genes, gene expression metrics (TPM), log-fold change relative to a reference cohort, and/or a tSNE map of index case). The first plurality of outputs may be generated based on (or according to) a detection of established cancer genes having aberrant gene expression in the tumor sample relative to that observed in normal control subjects (e.g., gene identifiers are intersected with known cancer genes from OncoKb to identify established cancer genes with aberrant gene expression). The first plurality of outputs may be generated based on (or according to) a prioritization of the detected aberrantly-expressed cancer genes in the tumor sample. In some embodiments, performing the RNA gene expression analysis may further comprise detecting over-expressed and/or under-expressed genes. The detection and/or identification of the over-expressed or under-expressed genes may be based on the TPM values satisfying a percentile threshold relative to the first reference database. For example, over-expressed and/or under-expressed genes can be flagged/detected, when a patient’s gene TPM is outside the 95% confidence interval of the cohort mean and/or standard deviation, and a fold change from the cohort mean is greater than or lower than 2 (or other values).

Referring now to operation (568), and in some embodiments, a DNA ploidy and allelic imbalance (e.g., deletions, loss of heterozygosity, and/or amplifications) analysis can be performed. The second dataset, the third dataset, and/or the third database can be used to perform the DNA ploidy and allelic imbalance analysis. In one example, a second plurality of outputs (e.g., a file containing prioritized allelic imbalances for reporting) can be generated (e.g., for the tumor sample) according to the performed DNA ploidy and allelic imbalance analysis. The second plurality of outputs can be generated based on (or according to) a detection of high-confidence aberrant copy number segments in the tumor sample (e.g., aberrant copy number segments are intersected with genomic coordinates of established cancer genes) by applying one or more allelic imbalance identification techniques (e.g., Battenberg and/or BRASS approaches). The second plurality of outputs can be generated based on (or according to) a prioritization of allelic imbalances in the tumor sample based on a set of criteria. The set of criteria may comprise an overlap of the high-confidence aberrant copy number segments in the tumor sample with the known cancer genes in the third database and/or other criteria. In some embodiments, the set of criteria for prioritizing allelic imbalances in the tumor sample can further include whole-genome duplication (WGD). In certain embodiments, the set of criteria for prioritizing allelic imbalances in the tumor sample may further include an aberrant copy number segment having a direction (e.g., aberrant segment direction) that is consistent with cancer gene function. For example, deletions and/or loss of heterozygosity in tumor suppressors (e.g., TP53), and/or amplification in oncogenes (e.g., MYC).

Referring now to operation (570), and in some embodiments, a variant calling analysis can be performed. Said variant calling analysis can be performed based (or by using) the RNA gene expression analysis of step 566 and/or the DNA ploidy and allelic imbalance analysis of step 568. In one example, a third plurality of outputs for the tumor sample can be generated based on (or as a result of) the performed variant calling analysis (e.g., somatic variant calling). The generation of the third plurality of outputs can be based on a detection of RNA fusions (e.g., by deploying independent fusion gene callers, such as FusionCatcher, STAR-Fusion, and/or Fu-Seq), a detection of somatic structural variants (e.g., by deploying independent structural variant callers, such as SvABA, BRASS and/or GRIDSS), a detection of somatic substitutions (e.g., by deploying substitution callers, such as CaVEMan, MuTect2 and/or Strelka2), a detection of somatic insertions and deletions (indels) (e.g., by deploying independent variant callers, such as Pindel, MuTect2, and/or Strelka2), an assessment of microsatellite instability (e.g., by deploying MSISensor) and/or mutational burden across variant classes, a detection of germline variants (e.g., by deploying independent germline callers, such as Strelka2 germline and/or FreeBayes), a clonality analysis, a determination of a number of structural variants and gene fusions in the DNA of the tumor sample (e.g., as compared against a cohort) and/or a determination of somatic mutation patterns or signatures in the tumor sample. The clonality analysis may comprise using purity and local copy numbers to scale a variant allele frequency (VAF) of single nucleotide variants (SNVs) and indels to cancer cell fraction (CCF) for one or more tumor samples from the patient. Candidate driver mutations, aberrant copy number segments, and/or structural variants may be assigned to each clone (e.g., to generate clone-specific mutation profiles).

In certain embodiments, the variant calling analysis may comprise detection of RNA fusions (e.g., see Step IV.A). The detection of RNA fusions may comprise employing a plurality of independent fusion gene callers on raw data (e.g., FusionCatcher, STAR-Fusion, and/or Fu-Seq). In some embodiments, the detection of RNA fusions may comprise a detection of high-confidence fusion genes. In one example, the detection of RNA fusions can include employing a rescue process (e.g., to detect lowly expressed fusions in established cancer genes). The rescue process may recover and/or identify detected high-confidence fusion genes that were not detected by at least two independent variant callers as a reference for known cancer genes (e.g., using FusionCatcher db as a reference for known cancer genes). The rescued fusions may be required to have at least one spanning read. In some embodiments, the variant calling analysis may comprise detection and/or identification of somatic structural variants (e.g., see Step IV.B). The detection of somatic structural variants may include deploying a plurality of independent structural variant callers (e.g., SvABA, BRASS, and/or GRIDSS) on raw data. In certain embodiments, the detection of somatic structural variants may comprise a selection of high-confidence structural variants. The selection of high-confidence structural variants may comprise merging all calls having more than a first predetermined number of base pairs (bp) (e.g., 600 bps) by a window (e.g., 100 bp window) that includes a breakpoint. The window may have a size (e.g., 100 bp) that is a second predetermined number of bps. The merged calls can be annotated a tool for annotating structural variants and the genes hit by them, such as AnnotSV.

In some embodiments, the variant calling analysis may comprise a detection and/or identification of somatic substitutions (e.g., see Step IV.C). The detection of somatic substitutions can include employing a plurality of independent substitution callers (e.g., CaVEMan, MuTect2, and/or Strelka2) on raw data (e.g. to retrieve high-confidence passed VCF). In certain embodiments, the variant calling analysis may comprise a detection of somatic indels (e.g., see Step IV.D). The detection and/or identification of somatic indels may comprise generating one or more indel signatures. In one example, the detection and/or identification of somatic indels may comprise using the one or more indel signatures to determine if a somatic indel is a repeat-mediated deletion, a microhomology association, and/or an insertion (e.g. to help better diagnose DNA repair deficiencies). In some embodiments, the variant calling analysis may comprise an assessment of microsatellite instability and/or mutational burden across variant classes (e.g., see Step V). In one example, the variant calling analysis can include determining a structural variant (SV) burden (e.g., tumor burden). The SV burden can be determined by collapsing complex structural variants into unique structural variant clusters to avoid over estimation of structural variant burden. In some embodiments, the variant calling analysis may comprise a detection of germline variants (e.g., see Step VI). The detection of germline variants can include deploying a plurality of independent germline callers (e.g., Strelka2 and/or FreeBayes) on raw data.

Referring now to operation (572), and in some embodiments, a workflow can be implemented. In one example, the workflow may comprise identifying orthogonal supportive indicators. The orthogonal supportive indicators can be identified based on consistency of two or more outputs in at least two of the first, second, and/or third pluralities of outputs (e.g., generated in step (566), step (568), and/or step (570), respectively). In one example, the workflow may comprise prioritizing genetic alterations based on the orthogonal supportive indicators. In certain embodiments, the workflow may comprise generating global classifications based on the orthogonal supportive indicators. The workflow may include classifying at least one somatic mutation in an established cancer gene. The established cancer gene may be detected and/or identified in both the first dataset and the second dataset as being orthogonally validated.

Referring now to operation (574), and in some embodiments, cohort classification scores may be generated and/or determined. The cohort classification scores may be generated for each individual level output in each of the first, second, and/or third pluralities of outputs for the tumor sample. In one example, the cohort classification scores may be generated based on (or according to) the reference cohort of tumor samples in at least one of the first reference database and/or the second reference database. In certain embodiments, generating the cohort classification scores can further comprise interrogating the at least one orthogonally-validated somatic mutation against a fourth database. The fourth database may associate, map, and/or relate a plurality of somatic mutations with a plurality of specific cancer types or pan-cancer markers. In some embodiments, generating the cohort classification scores can further comprise identifying the at least one orthogonally-validated somatic mutation as associated and/or related with a specific cancer type or pan-cancer hotspot (e.g., when there is a match). In some embodiments, generating and/or determining disease-specific classification scores further comprises performing a t-distributed stochastic neighbor embedding (tSNE) analysis.

Referring now to operation (576), and in some embodiments, disease-specific classification scores can be generated and/or determined. The disease-specific classification scores may be generated for each individual level output in each of the first, second, and/or third pluralities of outputs for the tumor sample. In one example, the disease-specific classification scores may be generated based on a subset of the reference cohort of tumor samples in at least one of the first reference database and/or the second reference database. In certain embodiments, the subset of the reference cohort of tumor samples can be of a same cancer type.

Referring now to operation (578), and in some embodiments, a report can be generated. The report may include the prioritized allelic imbalances, the microsatellite instability and/or mutational burden across variant classes (e.g., microsatellite instability score and status), the germline variants (e.g., germline mutations in established cancer predisposition genes), outputs of the clonality analysis, the number of structural variants and gene fusions in the DNA, the somatic mutation patterns or signatures, the at least one orthogonally-validated somatic mutation, the cohort classification scores, and/or the disease specific classification scores for the tumor sample. The report can be provided to one or more users for determination of an anti-cancer therapy (580). In one example, the report can be provided to the one or more users by transmitting, sending, and/or communicating the report to a computing device. The report can be provided to the user(s) by displaying, depicting, and/or illustrating the report on a display screen. In one example, the report can be provided to the user(s) by storing and/or maintaining the report in a non-volatile computer-readable storage medium. In yet another example, the report can be provided to the user(s) by printing (or sending to a printer for printing) the report onto a suitable printing medium.

In some embodiments, germline mutation pathogenicity can be classified and/or categorized by integration of data derived in step (566) relating to acquired somatic mutation patterns and/or signatures. In certain embodiments, the anti-cancer therapy can be determined based on values used for the report. In one example, the anti-cancer therapy can be provided, indicated, included, and/or specified via the report. In some embodiments, the anti-cancer therapy can be determined based on (or according to) an interrogation of an external therapy database. The interrogation of the external therapy database may identify and/or determine a therapy that aligns with the outputs in the report (e.g., outputs of the clonality analysis, somatic mutation patterns or signatures, and/or other outputs).

### Feasibility of Whole Genome and Transcriptome Profiling in Pediatric and Young Adult Cancers

For patients with pediatric or rare cancers that have low mutation burden and are primarily driven by structural variants and fusion genes, NGC-based panel tests may fail to identify a clinical biomarker (in most cases). Embodiments of the systems and methods described herein use said systems and methods (e.g., cWGTS) to enable the assessment of the full spectrum of germline and somatically acquired mutations, SVs, and/or CNAs, along with quantification of tumor mutation burden (TMB) and genome-wide mutational patterns in the context of pediatric, adolescent and young adult solid tumor patients with rare cancers.

### i. Sample Processing

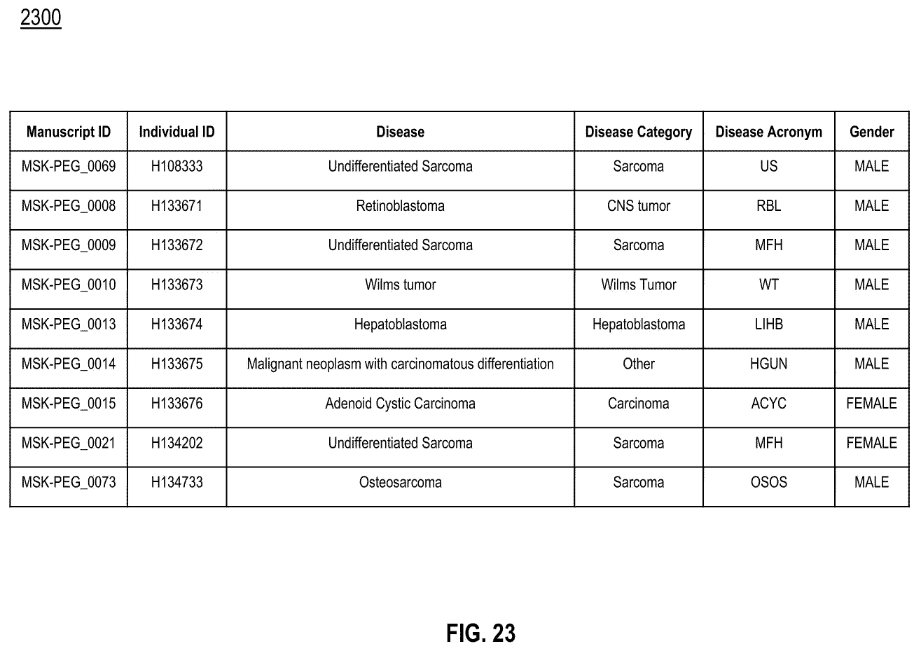

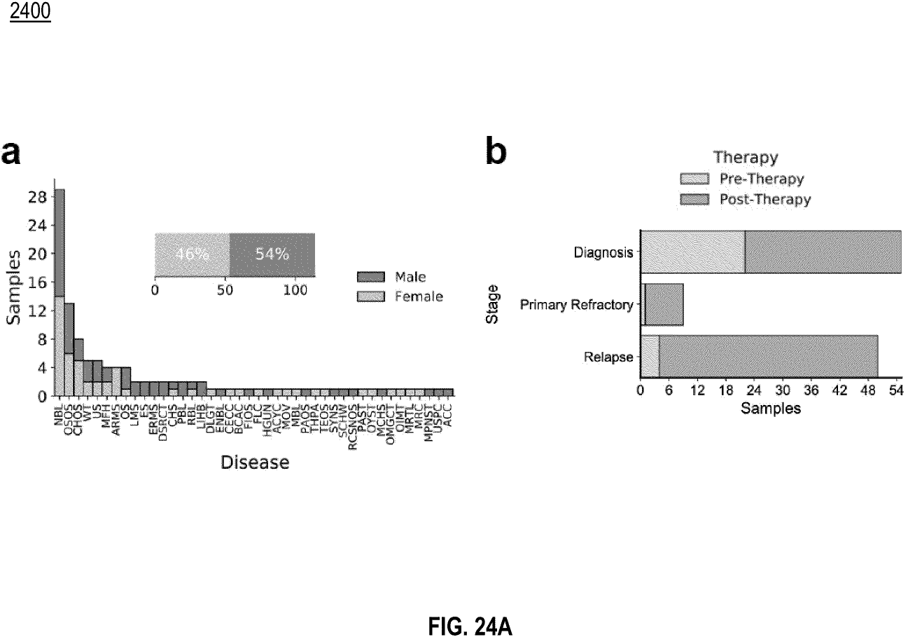

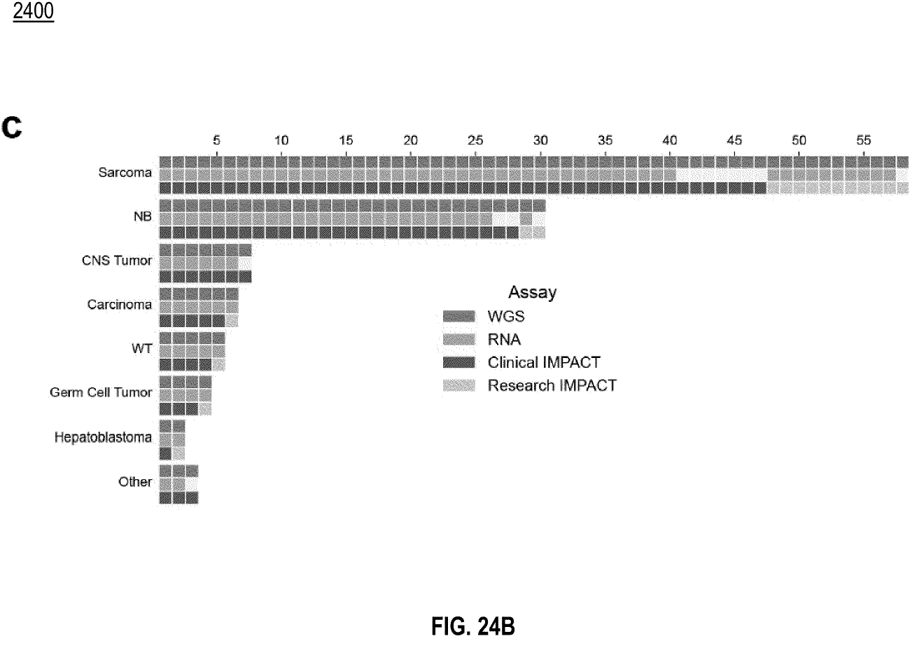

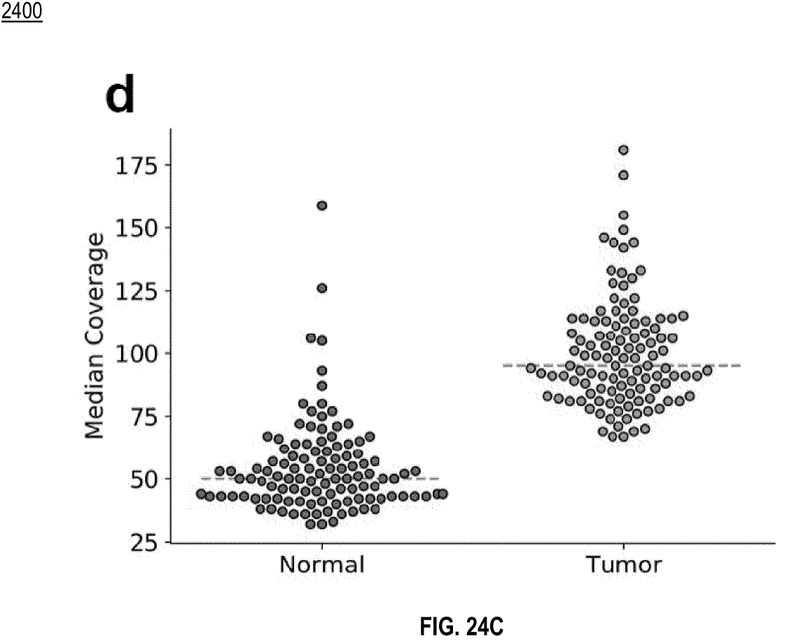

Of 201 patient fresh frozen (FF) samples nominated for paired cancer/normal whole genome and transcriptome sequencing (cWGTS), 87 cases fail to meet a requirement for >20% (or other percentage values) tumor purity by pathology review (n=58) or tumor purity assessment in sequencing data (n=29). The final cohort can include a single sample each from 114 pediatric adolescent and young adult patients (e.g., median age = 12.6 years, range: 4.5 months to 43.8 years) with solid tumors. Information of at least a portion of the final cohort can be seen in table 2300 of FIG. 23 and FIGS. 24A-24C. In addition to columns for Manuscript ID, Individual ID, Disease (e.g., Undifferentiated Sarcoma or Retinoblastoma), Disease Categry (e.g., Sarcoma or CNS tumor), Disease Acronym (e.g., US or RBL), Gender (e.g., Male or Female), such a table may additionally include columns such as Age in Days, Therapy (e.g., “Post Therapy” or “Pre-Therapy”), Stage (e.g., Relapse or Diagnosis), Disease Class (e.g., Local Recurrence, Metastasis, or Primary), DNA ID (e.g., I-H-108333-T2-2-D1-1), RNA ID (e.g., I-H-108333-T2-1-R1-1), Matched Clinical IMPACT (e.g., True or False), Matched Research IMPACT (e.g., True or False), and/or MSK-IMPACT Reseq (e.g., Yes or No). This level of attrition can highlight the need to optimize processing of FF biospecimens, exploring methodologies such as laser capture microdissection to enrich tumor cells, as well as develop optimized sequencing protocols for FFPE and/or other sources of tumor material such as cfDNA.

### ii. Implementation of a cWGTS Workflow for Clinical Decision Support

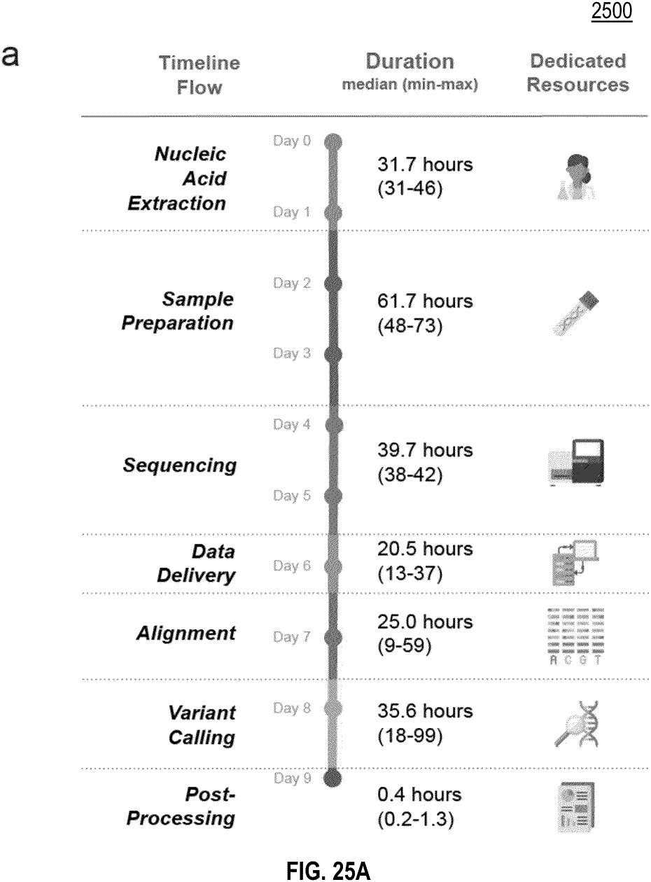

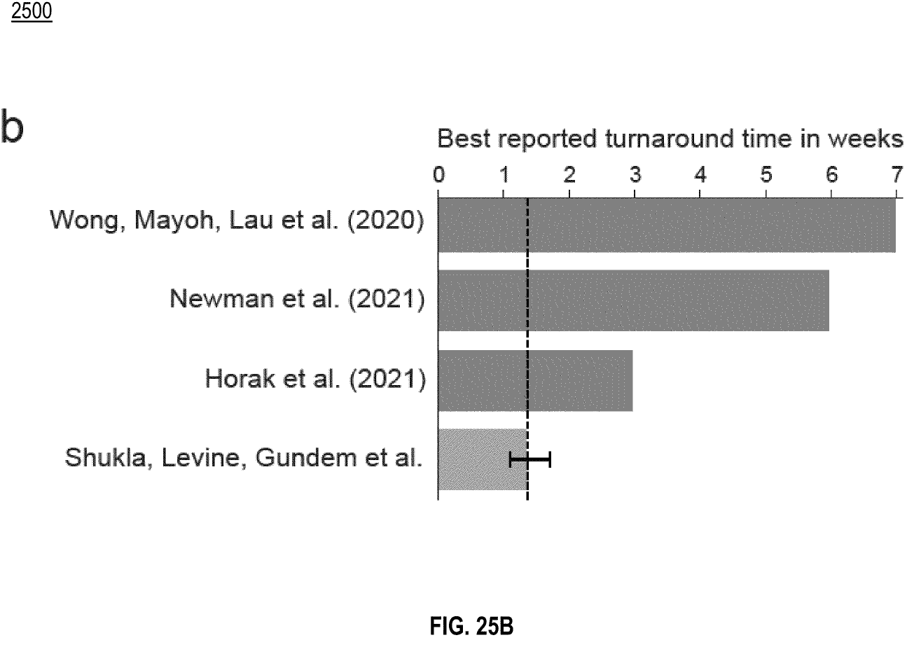

To prototype a clinical cWGTS workflow, an end-to-end process can be developed (as seen in FIG. 25A). The end-to-end process can include a dedicated: 1. Project management team; 2. Lab operators for sample processing; 3. Sequencing machines for cWGTS; 4. Data import channel; 5. Biosciences platform for automated deployment of analysis pipelines and API integration with institutional and public databases; 6. Reserved computing nodes in a high performance computing environment; and/or, 7. Systematic pipeline for prioritization and reporting of genomic findings (as shown in panel (a) of FIG. 25A). The end-to-end time from sample acquisition to the generation of an automated report for four consecutive batches (e.g., n=16 samples) can be quantified. Time logs can be audited starting at the time of surgical biopsy submission to report delivery for review by an interdisciplinary molecular tumor board. End-to-end, the optimized workflow of FIG. 25A can be executed in 9 days on average (as shown in panel (b) of FIG. 25B), which is shorter than the turnaround time for clinical NGS panel sequencing tests (e.g., 2-4 weeks) and markedly faster to WGS processing time frames in literature (e.g., 3-8 weeks, as seen in FIG. 25B). The results depicted in FIGS. 25A-25B demonstrate for the first time the feasibility of implementing cWGTS profiling to support diagnosis and treatment decisions with a clinically meaningful turnaround time.

### iii. Comprehensive Genome Characterization Utilizing cWGTS

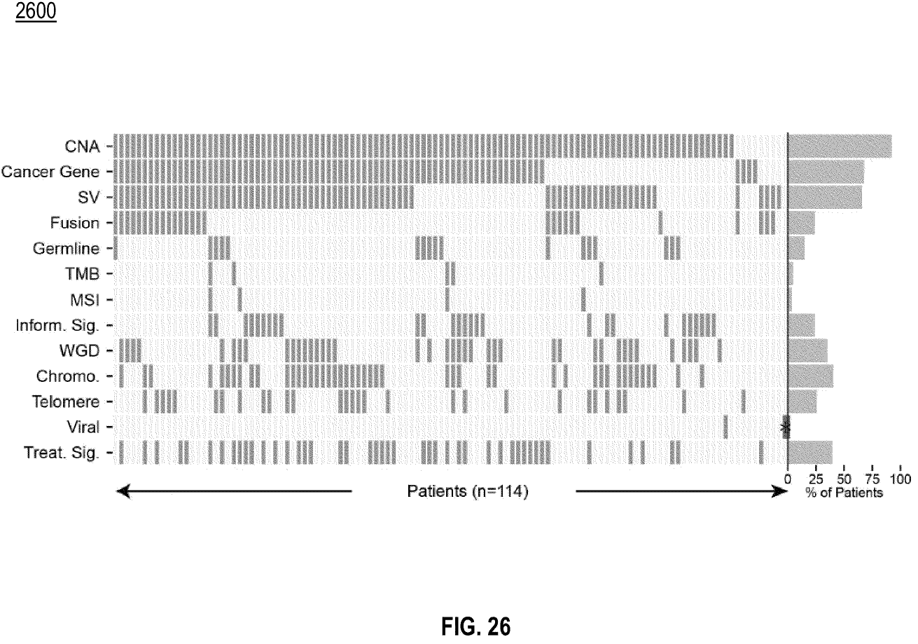

Across all mutational classes, cWGTS may identify on average 7,353 acquired mutations per sample, including cancer-associated alterations in 99% (e.g., n=113) of patients (as seen in FIG. 26). The acquired mutations per sample may include CNAs (e.g., n=105 patients), germline predisposition (e.g., n=17), mutations in cancer-associated genes (e.g., n=77), translocations/fusion transcripts (e.g., n=27), disease-associated SVs (e.g., n=75), and/or outlier TMB or microsatellite instability (MSI) scores (e.g., n=7). At least a portion of the acquired mutations per sample can be seen in a table that includes columns for, for example, Individual ID, Disease, Copy Number, Cancer Gene, Fusion, SV, Germline, TMB, MSI, Germline Signature, Signature 19 for Differential Neuroblastoma Diagnosis, Treatment Signature, Viral, Telomere, WGD, Chromothripsis, Other Oncogenic Event, Informs Diagnosis, Novel Fusions, Germline Signature, Prognostic Findings, Therapy Defining, Clinical, Incremental Finding, Findings (e.g., NUTM1-MGA fusion,TERTp rearrangement), Clinically Validated Findings (e.g., NUT1-MGA), Explanation of Findings, and/or New Finding Type (e.g., Fusion, SV, or Signature). Further signals of interest may include the delineation of mutation signatures, detection of chromothripsis or whole genome duplication (WGD), diagnostically relevant viral sequences (e.g., EBV), estimation of telomere length, and gene expression signatures. The SV’s, most of which can only be detected using WGS, can represent the third most frequent class of genomic alterations.

### iv. Concordance Analysis of cWGTS to Targeted DNA and RNA Panel Tests

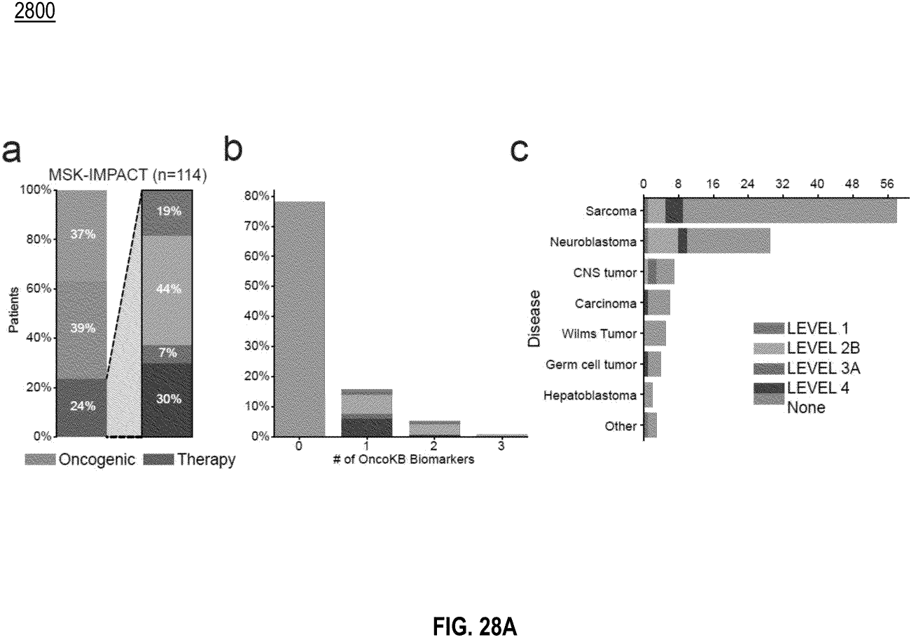

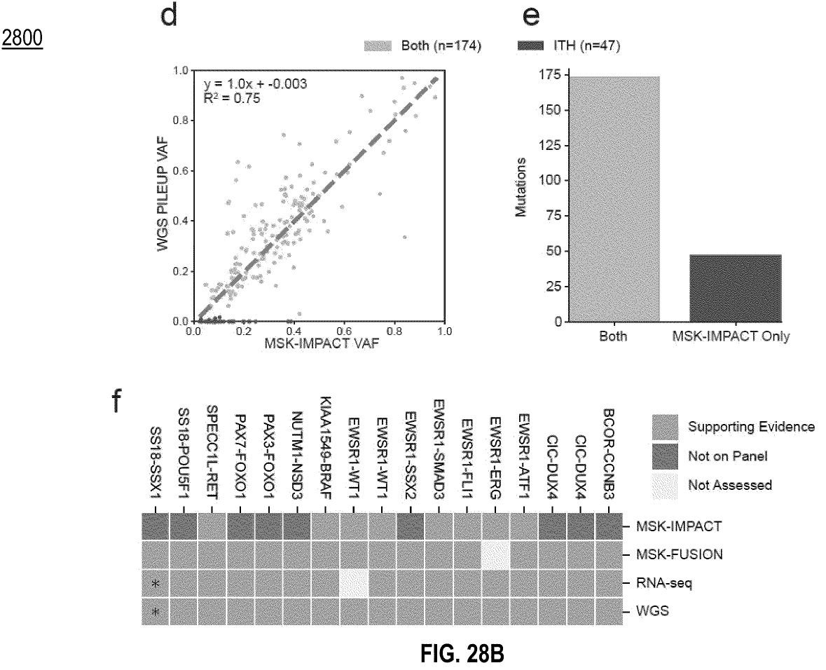

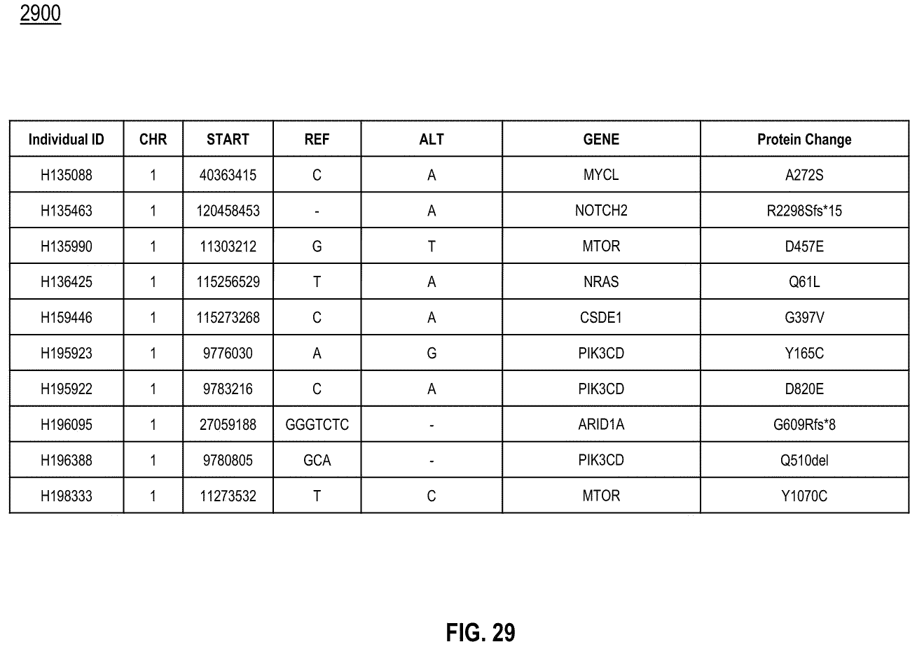

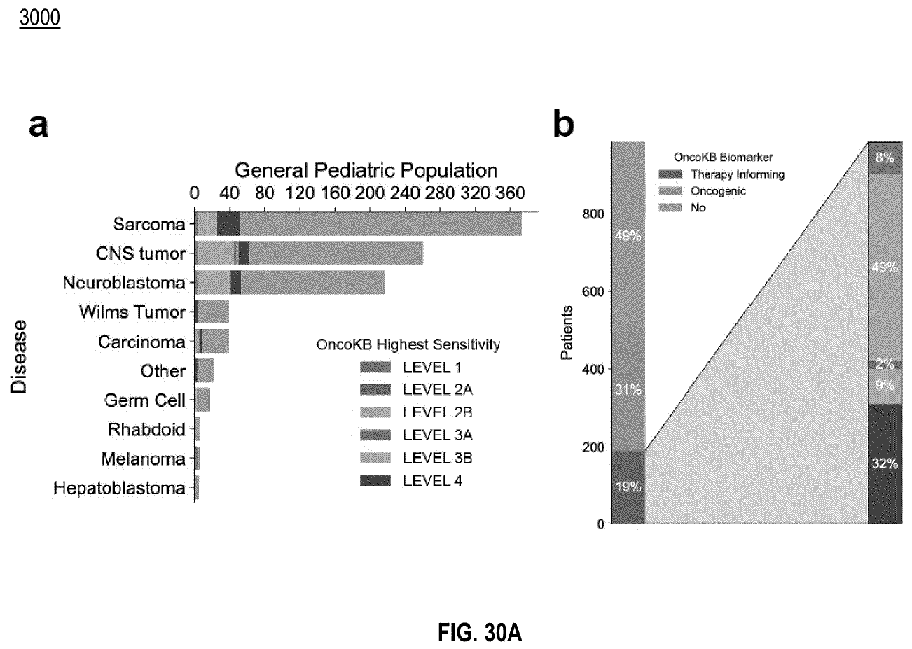

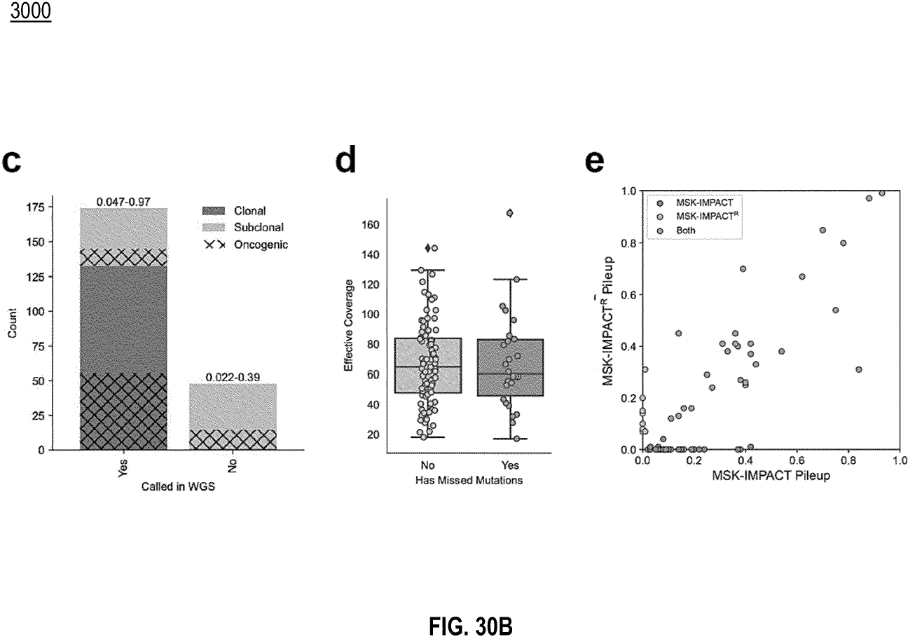

Within the cohort, targeted DNA profiling of corresponding formalin fixed paraffin embedded (FFPE) biopsies by MSK-IMPACT can detect actionable biomarkers as defined by OncoKb Levels 1-4 in a portion (e.g., 24%, n=27) of patients (as seen in panels (a)-(c) of FIG. 28A and table 2900 of FIG. 29). Table 2900 can illustrate sequencing metrics for at least a portion of the mutations reported by MSK-IMPACT (and corresponding data). For example, such a table can include columns for Individual ID, CHR, START, REF (e.g., C, G, GCA, GGGTCTC, or T), ALT (e.g., A, A, T, A, A, G, or A), GENE (e.g., MYCL, NOTCH2, or MTOR), Protein Change (e.g., A272S, R2298Sfs*15, or D457E), HGVSc (e.g., ENST00000397332.2:c.814G>T), OncoKb Oncogenicity (e.g., “Not Annotated,” “Likely Oncogenic,” or “Oncogenic”), Highest OncoKb Sensitive Level (e.g., None or LEVEL_3B), IMPACT VAF (e.g., 0.10 or 0.21), IMPACT Mutant Reads (IMPACT VAF*DEPTH) (e.g., 93 or 107), IMPACT Total Tumor Cell Reads (purity * depth) (e.g., 698.4 or 327.6), Proportion of mt/Total Tumor Depth (e.g., 0.13 or 0.33), IMPACT DEPTH (e.g., 970 or 520), IMPACT PURITY (e.g., 72 or 63), IMPACT CCF (e.g., 6.90 or 12.96), Called in WGS (e.g., True or False), WGS DEPTH (e.g., 100 or 92), WGS PURITY (e.g., 45.54 or 34.31), WGS Variant Reads (e.g., 0 or 16), WGS PILEUP VAF (e.g., 0.00 or 0.17), Expected Reads in WGS Corrected by WGS Purity (e.g., 3.14, 4.09, or 44.17), Expected Variant Reads (e.g., 9.59 or 18.93), Prop Test Corrected by Tumor Purity (e.g., 0.01 or 0.77), Significant (e.g., True or False), ITH Status (e.g., “Confirmed ITH” or “Both”), IMPACT CCF (e.g., 0.36 or 0.65), Subclonal (e.g., Yes or No), Called in RESEQ (e.g., True or False), RESEQ PILEUP VAF (e.g., 0.00), RESEQ PILEUP Reards (e.g., 2 or 0), RESEQ DEPTH (e.g., 607), and/or DISCREPANT CN (e.g., “MSK-IMPACT,” “No,” “WGS,” or “Both”). Consistent with prior findings demonstrating that patients with rare cancers do not yield clinically relevant biomarkers by panel sequencing, most patients in the cohort (76%, n=87) had no therapy-informing alterations. The results described herein may represent the expanded pediatric/young-adult patient population at MSK (see FIG. 30A).

In certain embodiments, an assessment of whether mutations captured by MSK-IMPACT were also detected by WGS can be performed. For discordant samples, MSK-IMPACT can be performed on the same DNA aliquots used to generate the WGS libraries. Performing MSK-IMPACT can enable a determination of whether discrepant calls were owing to differences in assay sensitivity (e.g., MSK-IMPACT and WGS) or a consequence of intra-tumor heterogeneity (ITH).

Of 221 somatic mutations reported by MSK-IMPACT, 174 (79%) were called in WGS (as seen in panels (d) - (e) of FIG. 28B). The called somatic mutations can include 68/83 (82%) mutations reported by MSK-IMPACT as oncogenic (as shown in panel (c) of FIG. 30B). Variants called by both assays can range from 5% to 97% (or other percentage values) variant allele frequency (VAF) with high concordance (e.g., r²=0.75) in VAF estimates (panel (d) of FIG. 28B). A majority of discordant mutations (e.g., 46/47) may be subclonal in MSK-IMPACT (<90% of cancer cell fraction) and 15 (or other numbers) can be classified as oncogenic (see FIGS. 29A-29F and panel (c) of FIG. 30B). Discordant mutations can present with a broad range of VAF (e.g., range: 2.2-39%, median=8.5%) as seen in FIG. 28B, and may show no systematic bias in effective coverage (see panel (d) of FIG. 30B). In some embodiments, 47 (or other values) discordant mutations can be confined to 26 samples (e.g., range 1-7 mutations per patient). Targeted re-sequencing of the WGS libraries by MSK-IMPACT may be performed for 44 (or other values) discordant variants and none can be called despite a median local depth of sequencing at 469x, supporting ITH as the basis of the discrepancies (as shown in panel (e) of FIG. 30B). Further corroborating ITH, targeted re-sequencing may identify 10 mutations (e.g., VAF: 6-31%) not reported by MSK-IMPACT in FFPE, of which 3 can be oncogenic (e.g., TP53 L265Yfs*81, PPM1D S468*, and HLA-A L102Hfs*73).

The validation studies described herein can demonstrate that discordant calls are due to stochastic sampling of heterogeneous tumors and in matched tumor biopsies cWGTS had 100% concordance with mutations detected by MSK-IMPACT.

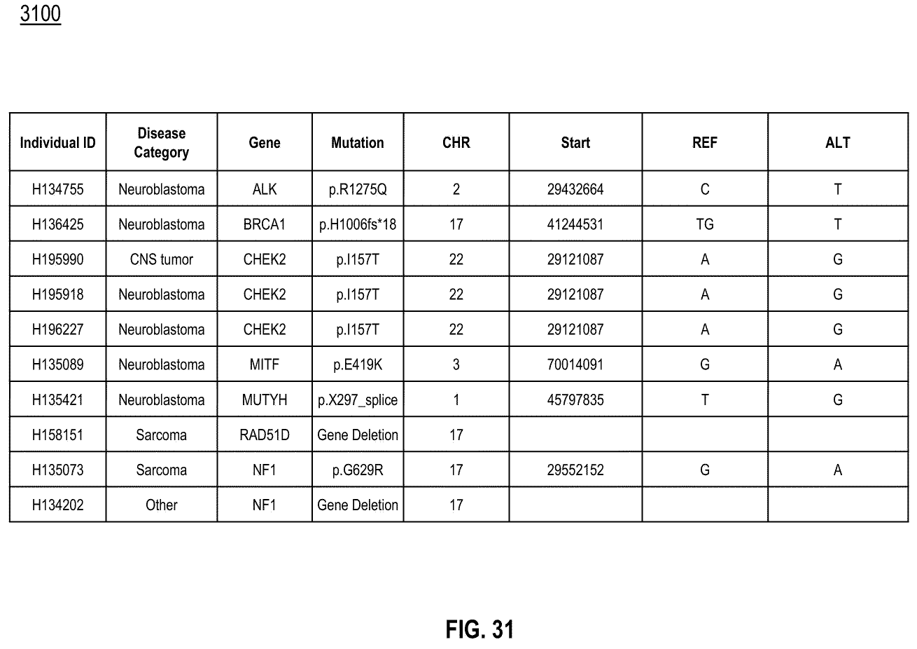

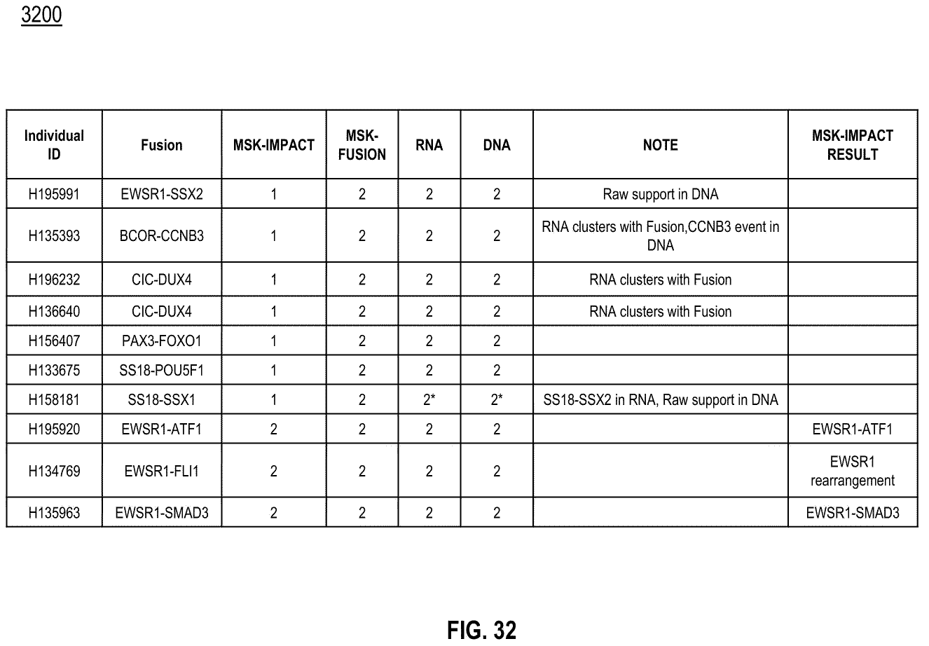

Germline assessment by MSK-IMPACT may identify predisposition variants in 13 patients. A panel RNA assessment with MSK-Fusion may identify oncogenic fusion genes in 18 patients (as seen in table 3100 of FIG. 31, table 3200 of FIG. 32, and panel (f) of FIG. 28B. Such tables may include columns for, for example, Individual ID, Disease Category, Gene, Mutation, CHR, Start, REF, ALT, HGVSc, VAF, Genotype, Reported By, Fusion, MSK-IMPACT, MSK-Fusion, RNA, DNA, Note, MSK-IMPACT Result, MSK-Fusion Results, Left RNA Breakpoint, Right RNA Breakpoint, Left DNA Breakpoint, Right DNA Breakpoint, and/or DNA Event). A cWGTS analysis may capture the germline predisposition variants (e.g., 13) and the fusions (e.g., 18). The fusion genes can be supported by data in both WGS and RNA-seq, which offers the opportunity to orthogonally validate findings within a single workflow (panel (f) of FIG. 28B).

The findings described herein can demonstrate that cWGTS as an integrative assay allows for the detection of germline, somatic mutations and fusion genes captured by an array of standard of care diagnostic tests

### v. Technical Considerations: Optimal Depth of Coverage for Clinical Sequencing

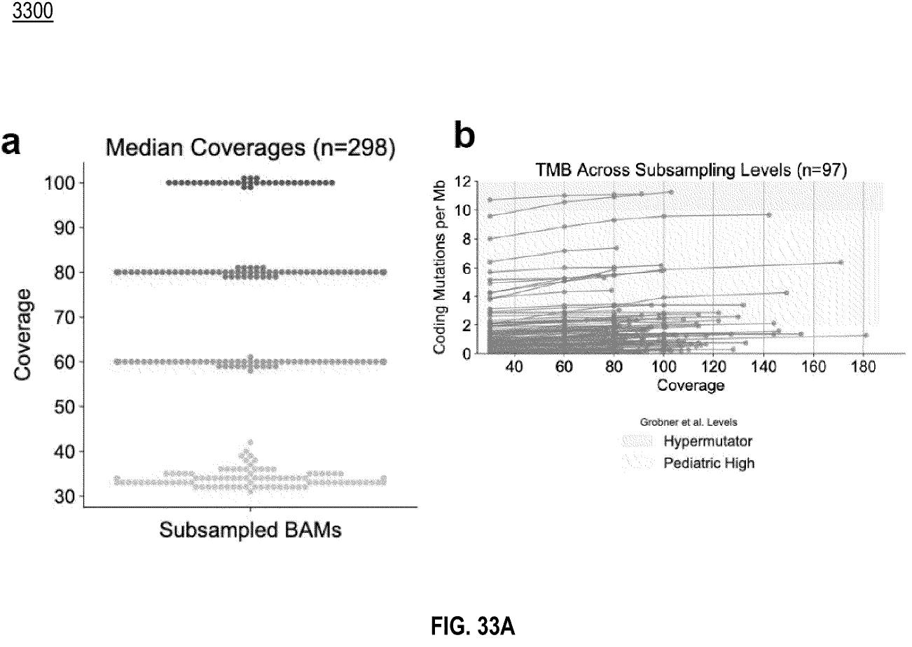

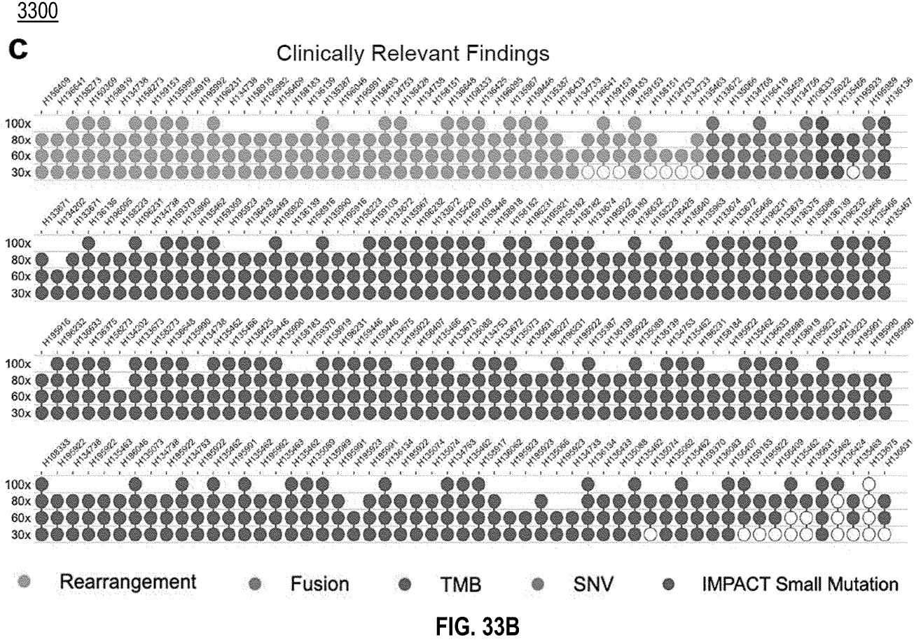

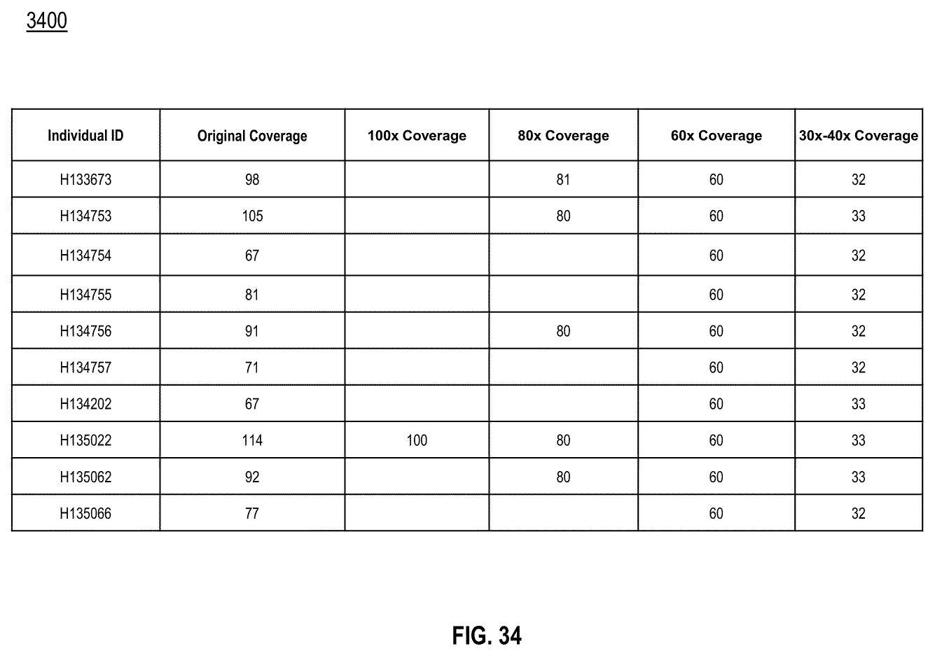

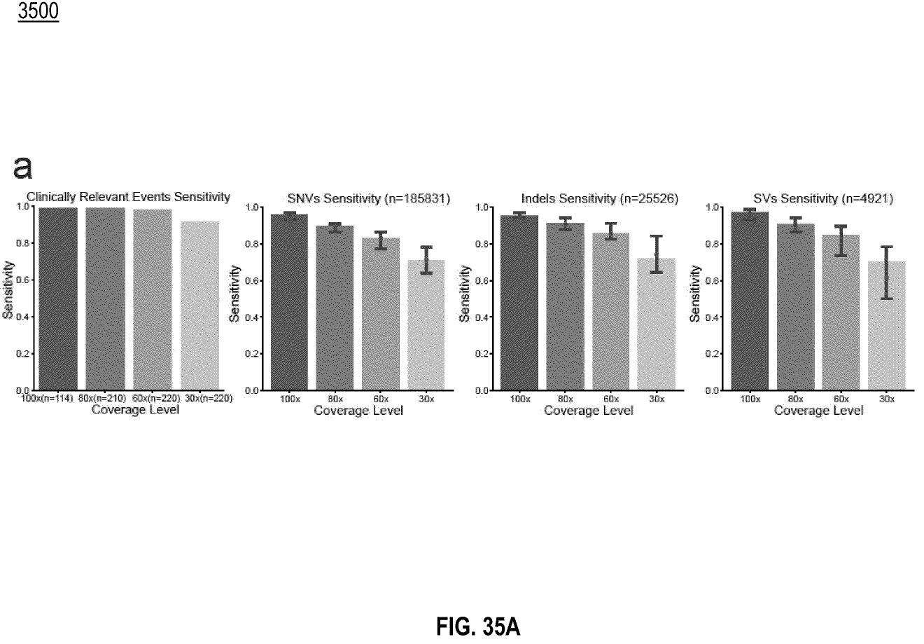

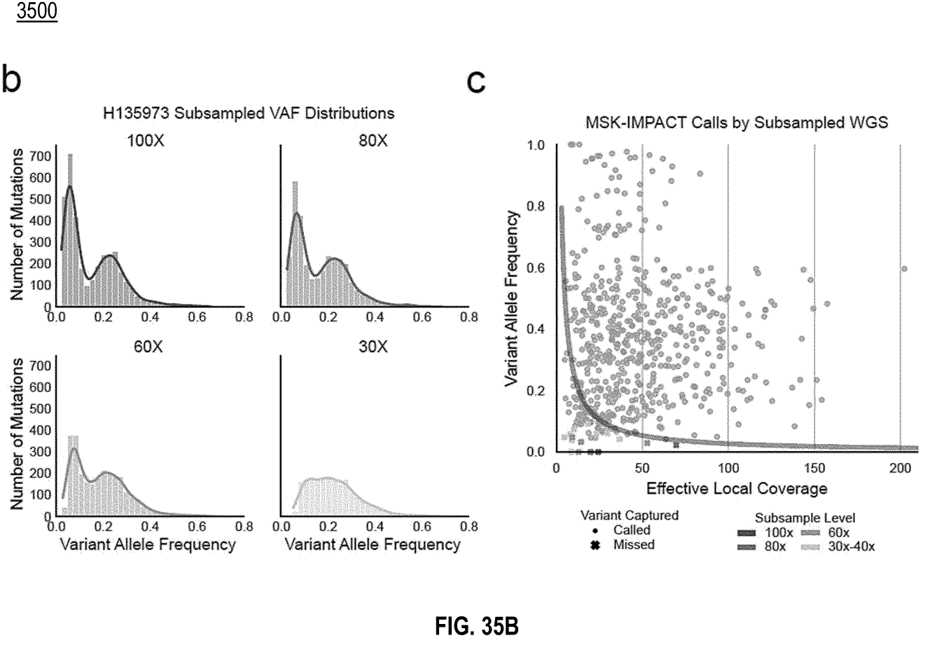

A sensitivity for somatic variant detection can be directly dependent on the tumor cellularity of the biopsy and/or the depth of sequencing coverage. The median cWGS depth may be 95x (e.g., range 67-181) and the tumor purity ranged from 21%-100%, resulting in a median effective coverage of 64x (e.g., median depth * purity estimate). To evaluate an optimal depth of sequencing, for each of 97 tumors with WGS coverage ≥60x (or other coverage values), 298 derivative sub-sampled BAM files can be generated in the range of 100x, 80x, 60x and/or 30-40x (as seen in panel (a) of FIG. 33A and a table that may include columns for, for example, Individual ID, Original Coverage, 100x Coverage, 80x Coverage, 60x Coverage, and/or 30x-40x Coverage). A median coverage for at least a portion of the cohort can be seen in table 3400 of FIG. 34. De-novo variant calling can be performed to assess the sensitivity of detection for clinically relevant findings by MSK-IMPACT and cWGTS (e.g., n=220), genome-wide mutations across variant classes and TMB (as shown in panel (a) of FIG. 35A and panels(b)-(c) of FIGS. 33A-33B). The detection sensitivity may correlate with the effective coverage, and can be affected by the variant class with slightly less sensitivity for SV’s (as seen in panel (a) of FIG. 35A). Of the oncogenic findings, >91% (or other percentage values) can be captured at 30-40x, and >98% (or other percentage values) may be re-called at 60-100x (as shown in panel (a) of FIG. 35A). With a lower sequencing coverage, the power to detect subclones can be limited (e.g., VAF range: 4.3-31%, median=9.5%) (as shown in panels (b)-(c) of FIG. 35B). An optimal sensitivity for genome wide mutation calling across variant classes can be attained at ≥80x and increased with coverage. Panel (c) of FIG. 35B can provide an overview of variant detection sensitivity by depth of sequencing coverage, tumor purity and/or variant clonal representation.

### vi. Additional Findings of Biological and Clinical Relevance Detected by cWGTS

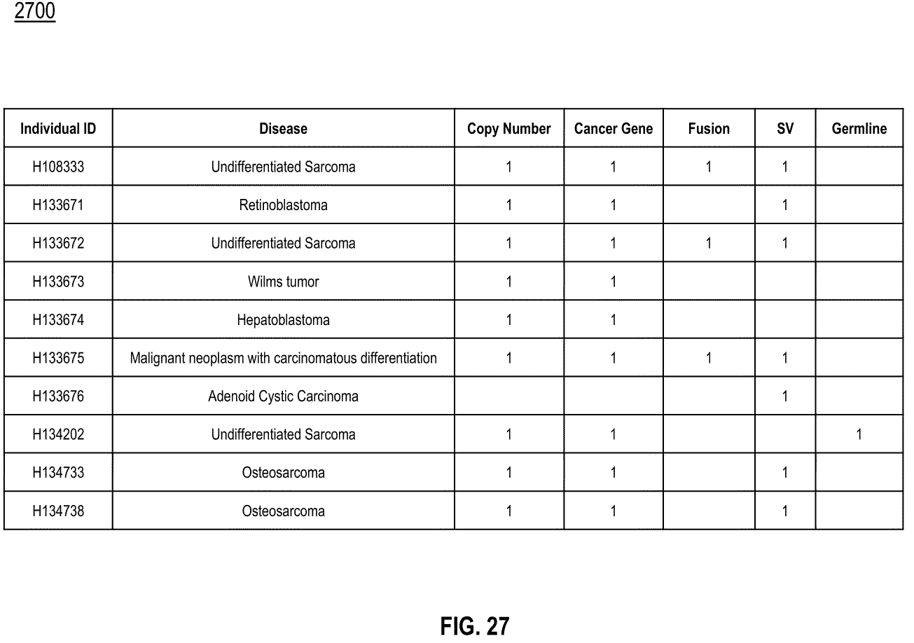

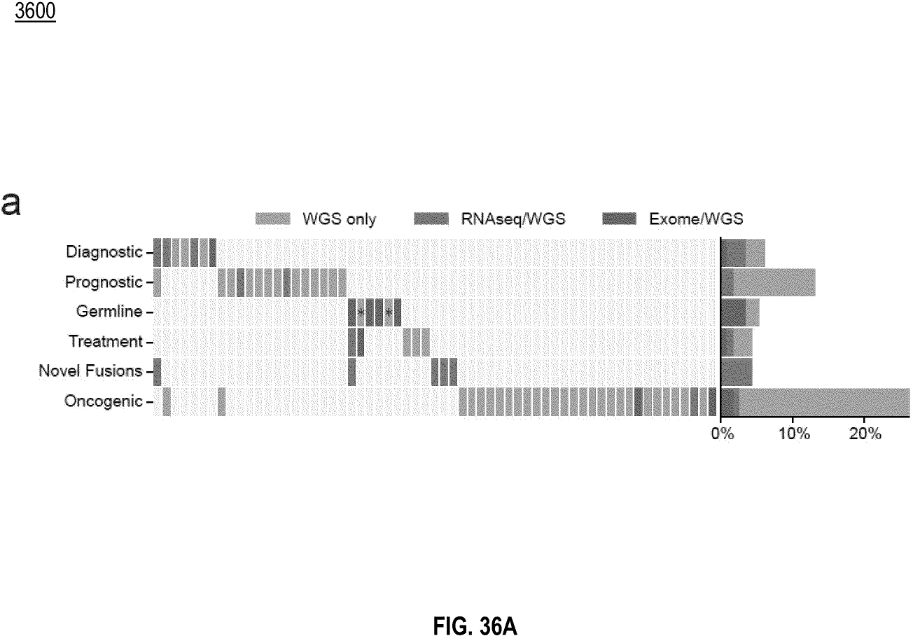

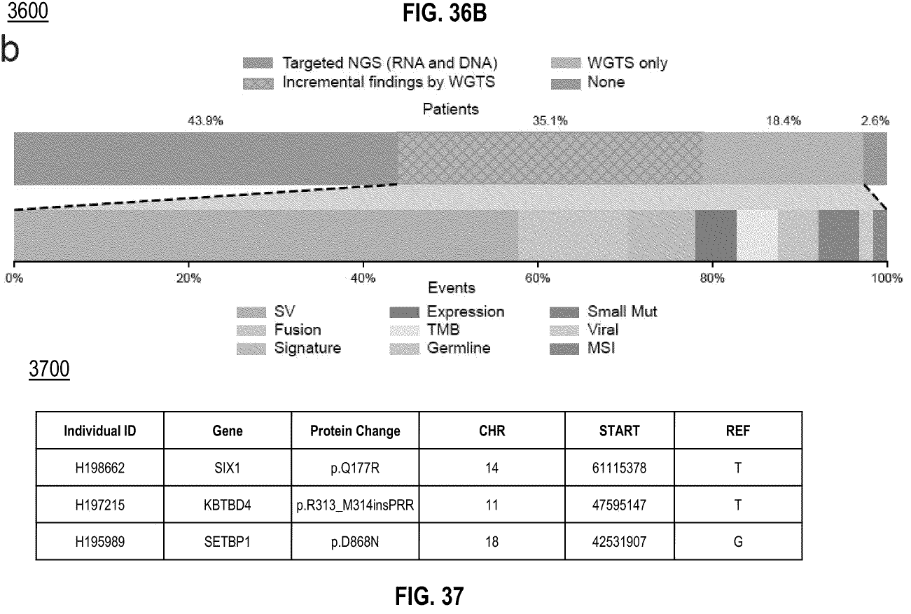

Additional findings of clinical relevance by cWGTS can be assessed over state of the art panel sequencing to include somatic MSK-IMPACT, MSK-fusion and/or panel testing of 88 cancer predisposition genes. Consistent with recent studies, the cWGTS analysis can identify at least one additional cancer-associated oncogenic variant in 54% (or other percentage values) of patients (n=61). Of the 54% of patients, 33 may be of direct clinical relevance including 12 diagnostic (36%), 15 prognostic (45%), 5 therapy informing (15%), and 6 germline (18%) biomarkers (as shown in panel (a) of FIG. 36A and table 2700 of FIG. 27). Most additional relevant findings can be explained by the detection of SVs, mutations in rare cancer genes, and/or genome-wide mutation signatures (as shown in panel (b) of FIG. 36B).

A portion of additional findings that would be captured by whole exome sequencing (WES), by masking results to the coding regions of genes, can be further inferred. Of the 61 patients with incremental findings by cWGTS, RNA-seq and WES alone may capture events in 10 (16%) and 8 (n=13%) patients respectively, or in 17 (28%) patients when combined (FIG. 36A). Thus, less than half of the findings in cWGTS may be captured by WES and/or RNA-seq.

### vii. Additional Findings: Rare Variants in Established Cancer Genes

At least seven clinically relevant mutations targeting known cancer-associated mutations in rare genes can be identified (as seen in table 3100 of FIG. 31 and table 3700 of FIG. 37, which can additionally include columns for, for example, ALT, HGVSc, VAF, DEPTH, CCF, and/or Mutation Copy Number). The somatic SNV/lndel driver mutations in cancer genes for at least a portion of the cohort can be seen in table 3700. Of the clinically relevant mutations, three can be somatically acquired and include a disease defining mutation of KBTBD4 (e.g., p.R313_M314insPRR) in a pineal parenchymal tumor of intermediate differentiation, a SETBP1 (e.g., p.D868N) mutation in a germ cell tumor, and/or a SIX1 mutation (e.g., p.Q177R) in a Wilms tumor. Additionally, clinically relevant germline variants may be detected in four cancer-associated genes including an SBDS splice site mutation in a rhabdomyosarcoma, BARD1 p.E652fs*69 in a neuroblastoma, EP300 p.A2259fs*20 and EXT2 p.W414* in two osteosarcoma patients (table 3100 of FIG. 31). The results can demonstrate the utility of cWGTS in capturing somatic and germline variants in rare cancer genes not routinely evaluated in targeted panels.

### viii. Additional Findings: Fusion Genes

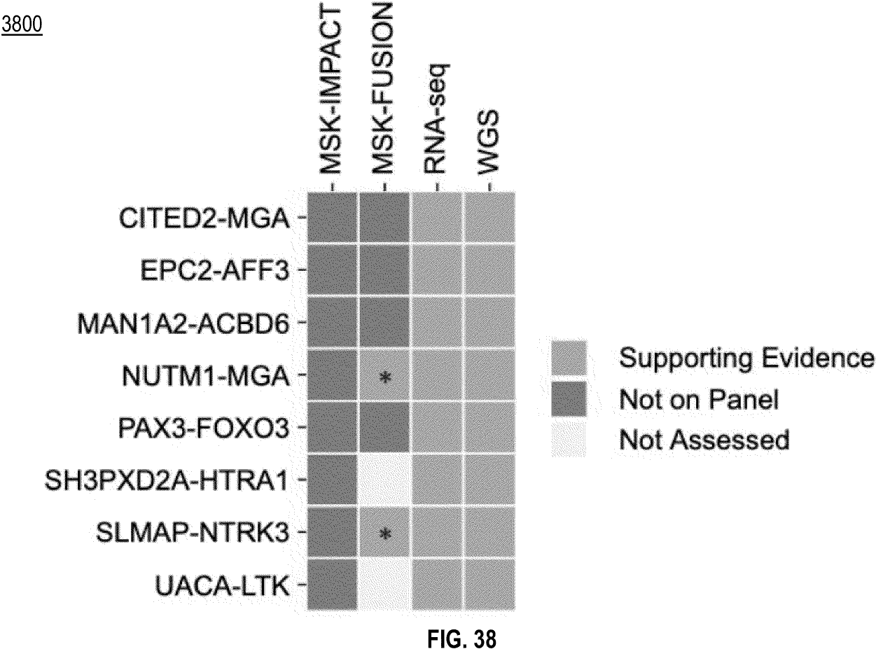

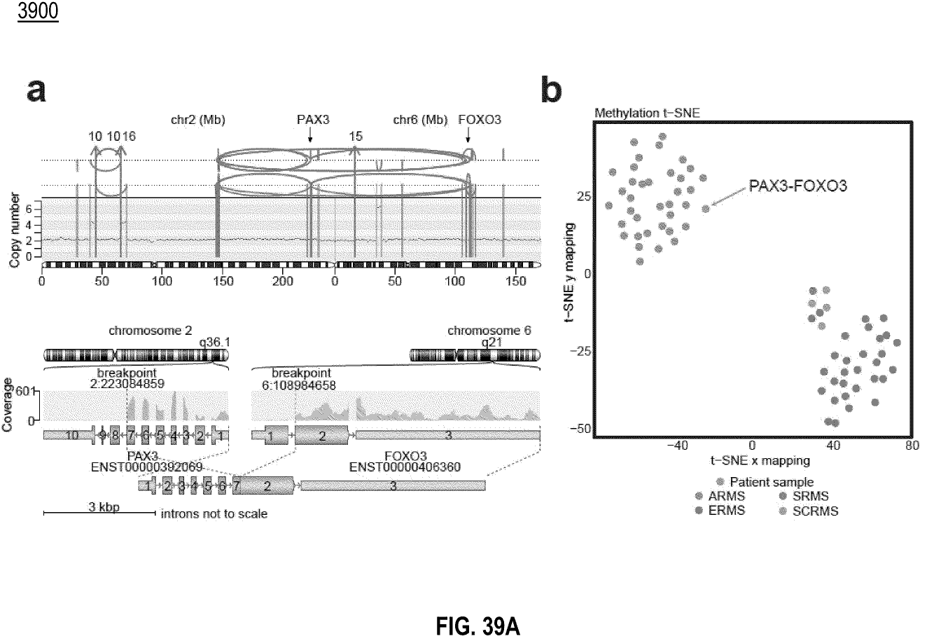

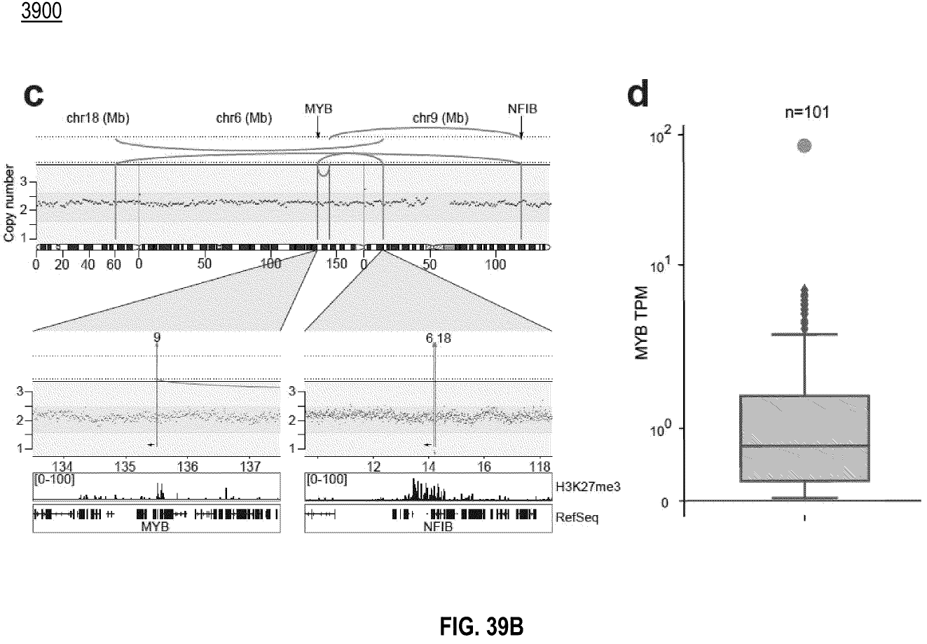

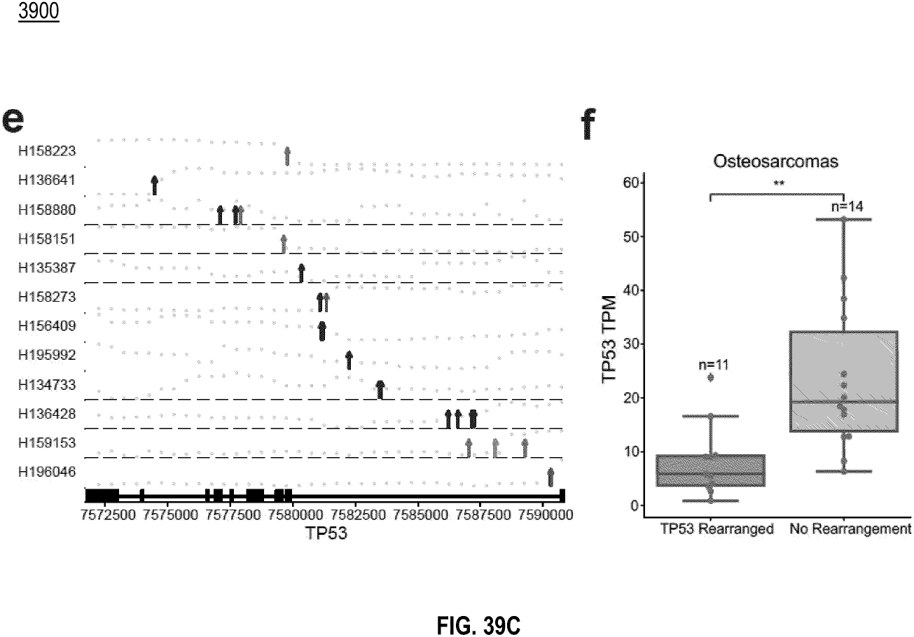

Eight in-frame fusion genes may be identified from WGS and RNA-seq in patients with no prior findings on clinical testing (as shown in FIG. 38 and table 3200 of FIG. 32A), 5 of which were novel. Of diagnostic relevance we identify: 1. a t(2;6) (PAX3-FOXO3) translocation changing diagnosis to alveolar rhabdomyosarcoma (ARMS) in a patient who was diagnosed with embryonal rhabdomyosarcoma (ERMS) in the absence of the cardinal ARMS fusions (e.g., PAX3-FOXO1 and PAX7-FOXO1) (as shown in FIG. 39A); 2. A UACA-LTKfusion in a metastatic papillary thyroid carcinoma; and 3. a pathognomonic SH3PXD2A-HTRA1 fusion establishing a diagnosis of schwannoma in a patient evaluated for relapsed stage IV neuroblastoma.

Of potential therapeutic relevance, a NTRK3-SLMAP fusion in a neuroblastoma patient is identified. Activating NTRK3 fusions may be promising therapeutic targets for TRK inhibitors, with activity seen across pediatric and adult cancers. However, screening for NTRK fusions is not routinely performed across all disease indications. Additional novel fusions included EPC2-AFF3 and MAN1A2-ACBD6 identified in two patients with undifferentiated sarcoma, and a CITED2-MGA fusion in a round cell sarcoma not otherwise specified (NOS).

### ix. Additional Findings: Structural Variants Targeting Tumor Suppressor Genes

Structural variations of established prognostic relevance can be observed in the cohort, providing insights of clinical relevance. A cWGTS analysis mapped events in TERT and ATRX in 8 (28%) and 5 (17%) neuroblastoma patients respectively. Both TERT and ATRX are increasingly considered as therapy-defining risk stratification biomarkers for neuroblastoma. The TERT SVs can be identified by cWGTS, and only ¼ of the ATRX deletions may be reported by MSK-IMPACT.

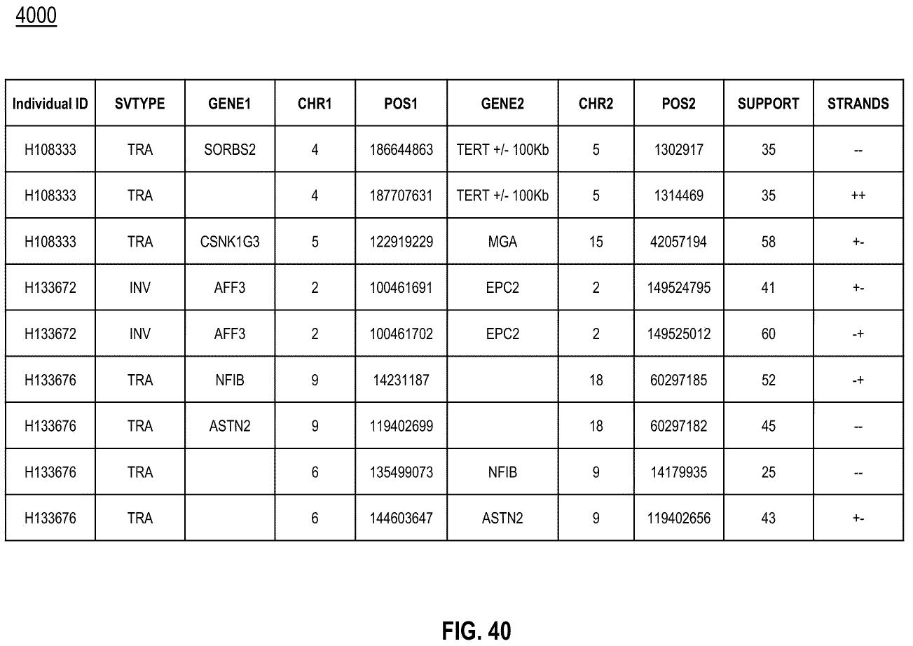

Recurrent SV’s targeting the tumor suppressor gene DLG2 can be observed in 15/29 OS patients and 3/29 neuroblastoma, of which 6 had homozygous deletions (see table 4000 of FIG. 40). The somatic SV driver mutations for at least a portion of the cohort can be seen in table 4000 of FIG. 40. Whilst DLG2 may be characterized in osteosarcoma, the findings described herein may demonstrate that DLG2 SVs are also recurrent in neuroblastoma warranting further investigation in future studies.

### x. Integration of RNA-seq and WGS for Variant Annotation

Interpretation of complex SVs that target non-coding regions of the genome presents a major challenge for reporting of WGS findings. cWGTS enables the concomitant detection of SV’s and assessment of the transcriptomic consequences of the affected loci. For example, a complex chain of SVs resulting in overexpression of the MYB oncogene through “hijacking” of an NFIB enhancer (as shown in FIG. 39B) was detected in an adenoid cystic carcinoma without informative clinical sequencing findings. While MYB overexpression is a cardinal feature of adenoid cystic carcinomas, MYB fusions are identified in only 30% of cases using conventional assays. Integration of gene expression data was critical to the annotation and reporting of this complex non-coding SV as the disease defining diagnostic biomarker.

Similarly, amongst 29 osteosarcoma patients, we identified TP53 mutations in 12 and mapped non-coding SVs targeting the TP53 locus in 13. Of these, only 3 were reported by MSK-IMPACT (as shown in panel (e) of FIG. 39C). Integration with RNA-seq demonstrated that TP53 SVs correlated with loss of TP53 expression, validating their functional relevance (as shown in panel (f) of FIG. 39C). Wildtype TP53 represents an inclusion criterion for TP53 pathway modulating drugs trials. Here, we show that in the absence of cWGTS, patients with loss of TP53 by SVs, which have been described in diverse cancers, could be erroneously diagnosed as TP53 wildtype with implications for assessment of treatment options. We did not identify germline SV’s targeting the TP53 locus.

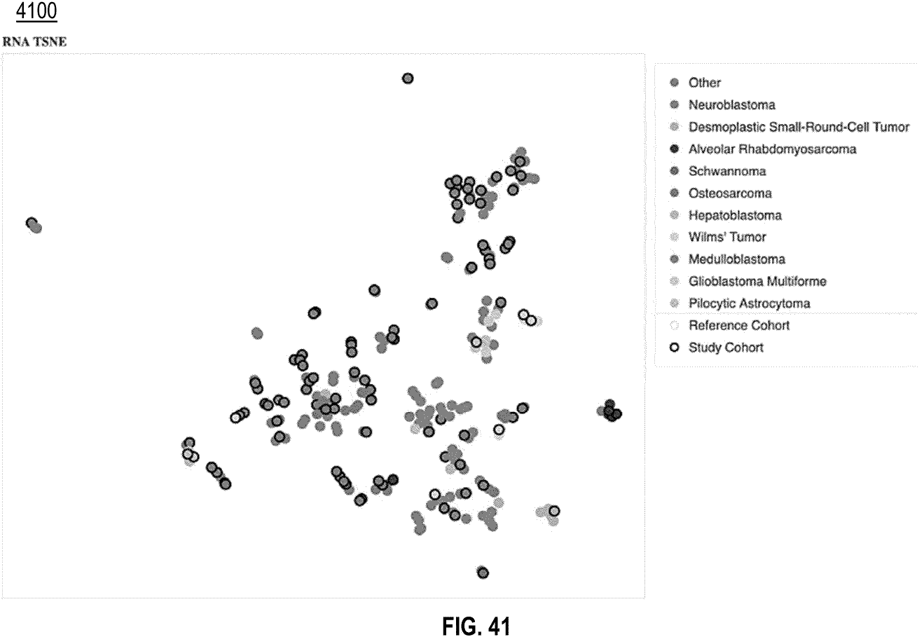

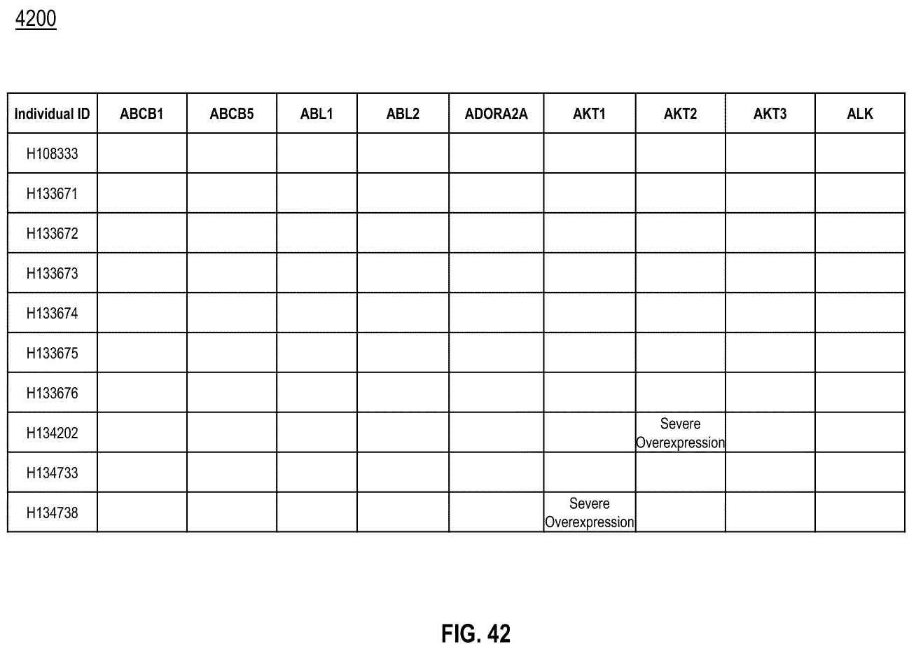

Our findings illustrate the necessity to combine RNA and DNA analyses in variant detection, annotation and prioritization for clinical cWGTS reporting. In our automated workflow, we interrogate DNA mutations for corroborating evidence in the RNA. All recurrent fusion genes reported by panel RNA-seq assays as well as the 8 additional driver fusion genes were orthogonally detected by both WGS and RNA-seq (as shown in FIG. 38 ). Integration of gene expression data to SV findings resolved functional consequences of SVs targeting non-coding regions on the genome (e.g., MYB enhancer hijacking and TP53 inactivation). Global gene expression signatures were further used to cluster samples by tumor type, providing further opportunity to resolve a patient’s diagnosis (as shown in FIG. 41). Last, RNA-seq identified on average 16 gene expression biomarkers per sample (at least a portion is shown in table 4200 of FIG. 42). However, the evidence with regards to the clinical utility of such expression biomarkers remains to be validated.

### xi. Integration of Germline Mutations to Somatic Mutation Signatures for Variant Annotation

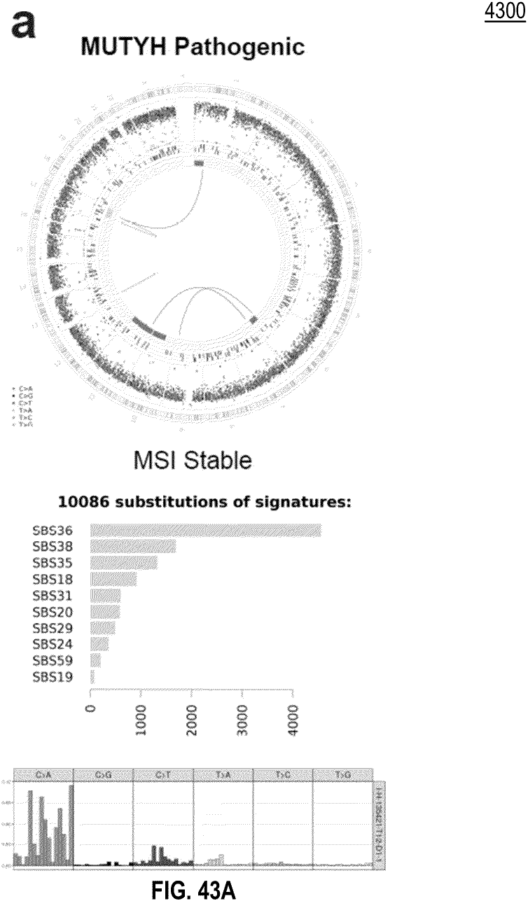

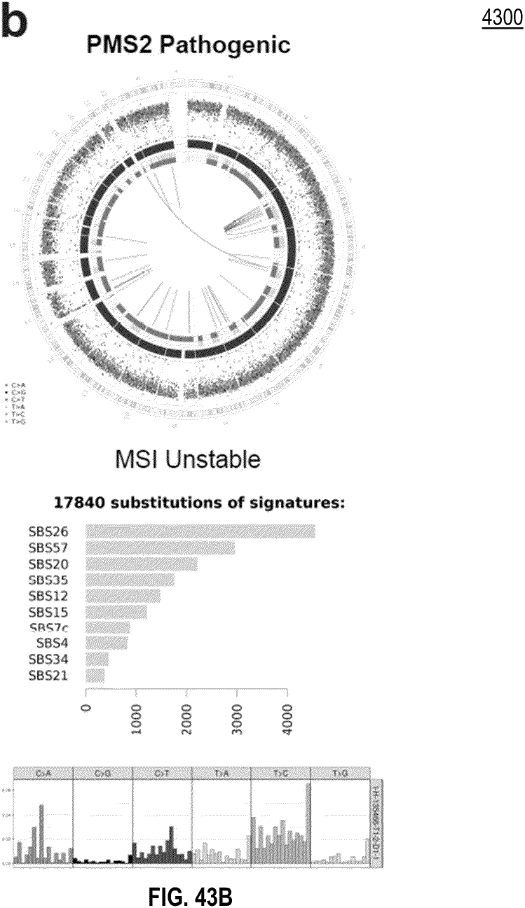

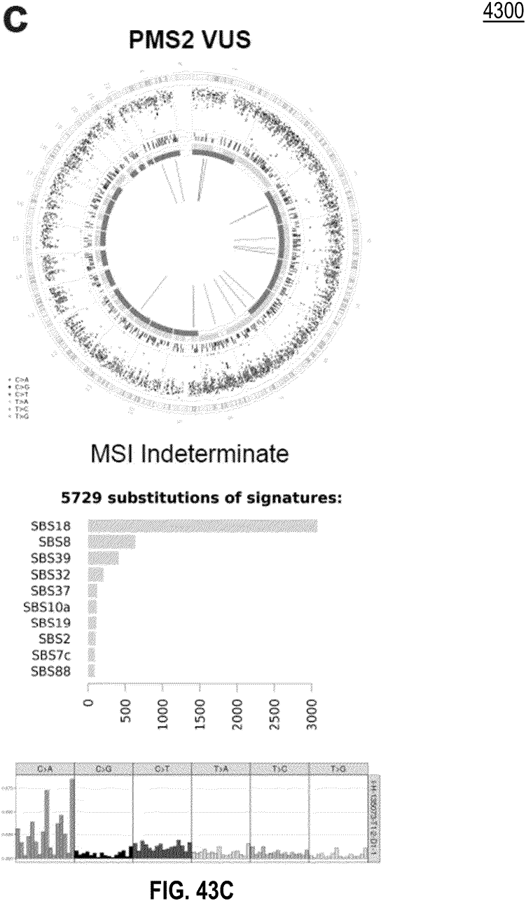

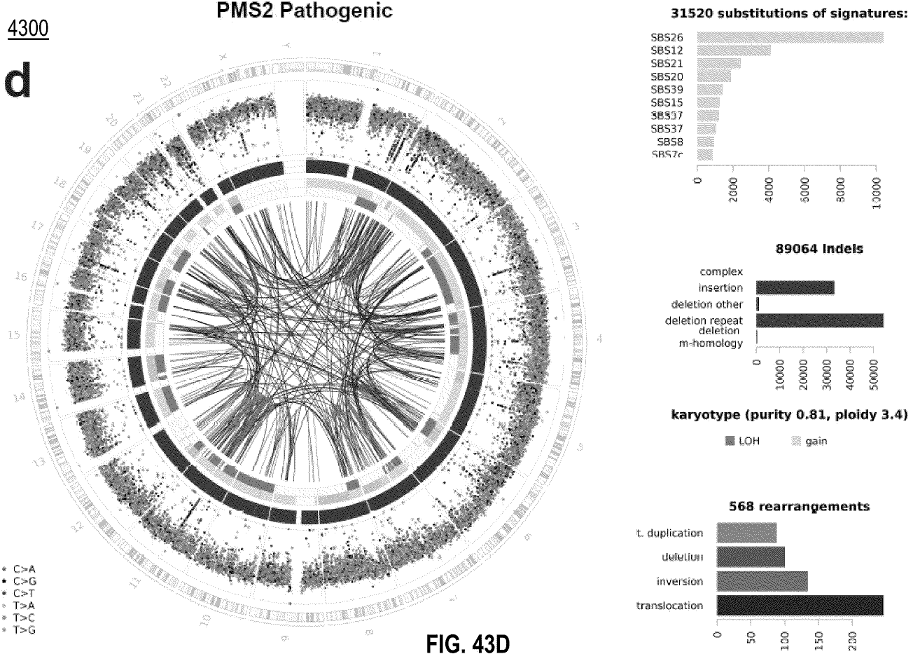

Annotation of germline variants is restricted to recurrent events in population databases, thus limiting interpretation for rare founder events. For each genome in our cohort, we quantified the proportion of mutations attributed to each of 73 reference mutation signatures. Two of three patients with a germline mutation in DNA repair genes further harbored mutation profiles suggestive of DNA repair deficiency. Patient H135421 had a pathogenic variant in MUTYH and somatic loss of the second allele. 42% of the mutations were attributed to the MUTYH signature SBS36 (as shown in FIG. 43A). Patient H135466 had a pathogenic variant in PMS2 (X180_splice) with loss of the wild-type allele by LOH. The tumor was MSI high with hypermutation (TMB=11.23, Indels=90,246, SNVs=17,840, SVs=44), enrichment of T>C mutations and repeat mediated indels characteristic of PMS2 deficiency (as shown in FIG. 43B). In contrast, Patient H135073 harbored a variant of unknown significance (VUS) in PMS2, a medium MSI score (7.23) and low mutation burden (1.30 Muts/Mb) without evidence of a PMS2 signature (as shown in FIG. 43C). These findings demonstrate the utility of mutation signatures in the assessment of germline mutations in DNA repair genes.

To illustrate this point, in a 12 year old osteosarcoma patient outside the study cohort, cWGTS characterized a hypermutated genome (TMB=16.7, Indel=89,588, SNVs=31,520, SVs=568) enriched in repeat mediated deletions consistent with MSI high status (as shown in FIG. 43D). This observation prompted consent for germline testing resulting in identification of a PMS2 mutation (p.D699H) annotated as likely pathogenic/VUS and a somatic loss of the wild type allele. MSK-IMPACT reported an indeterminate MSI status (7.5) yet upon testing validated the germline mutation is pathogenic.

These results demonstrate the utility of integrating composite readouts from cWGTS (germline mutation, allele specific copy number, genome wide TMB) to deliver corroborating evidence for the assessment and reporting of germline predisposition mutations with implications for family screening, diagnosis and treatment.

### xii. Genomic Alterations of Emerging Biological and Clinical Relevance

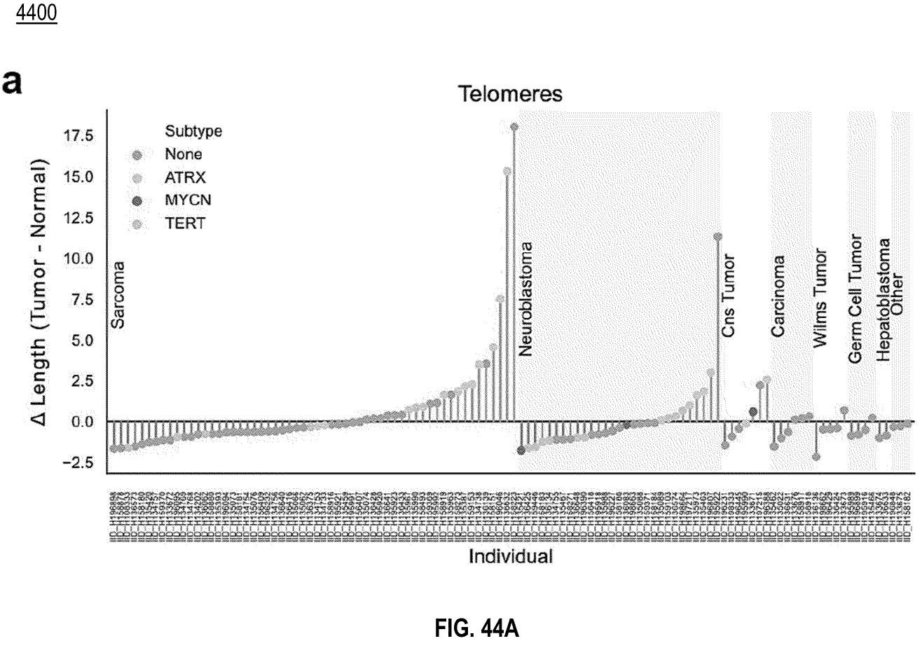

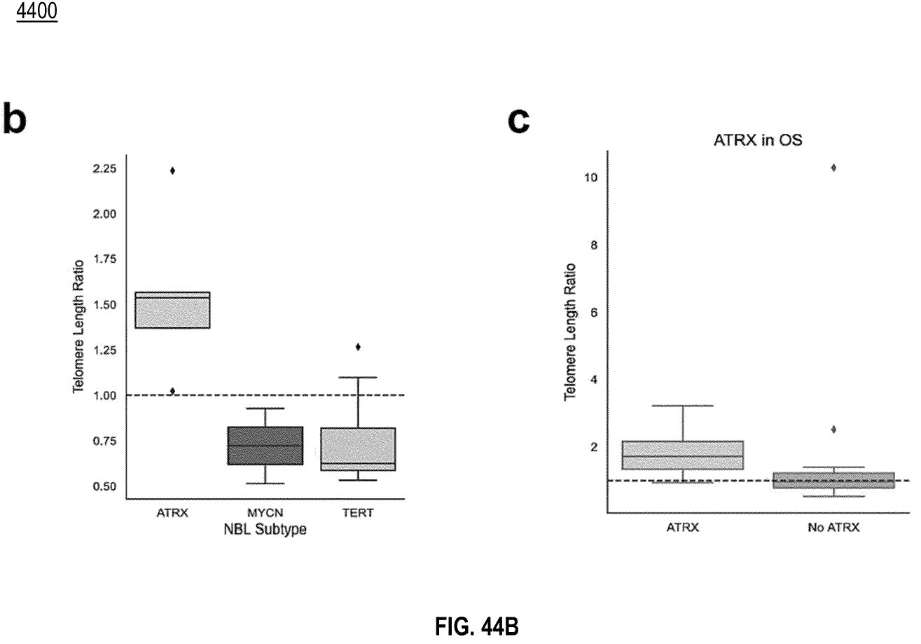

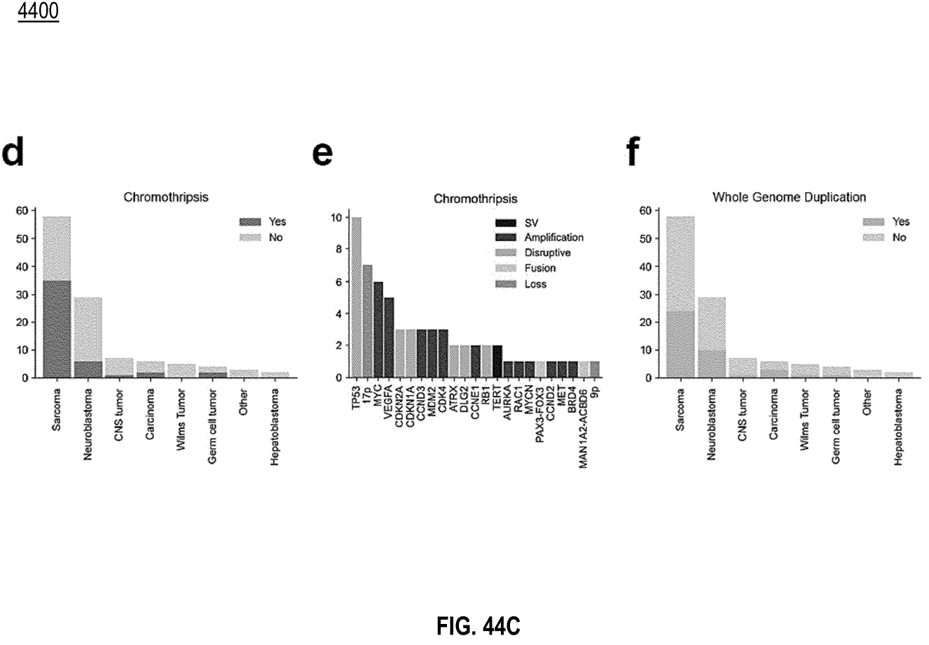

Recent studies propose telomere length as prognostic indicators in neuroblastoma amongst other cancers. We recapitulated the established associations between ATRX and TERT mutations to telomere length (as shown in FIGS. 44A-B). ATRX mutations were also observed in 8 osteosarcomas, with similar associations to telomere length (as seen in panel (c) of FIG. 44B). Given the association between adverse risk mutations and telomere length, delineation of the independent prognostic value warrants analyses of data that concomitantly map these mutations, SV’s and telomere length alongside established predictors of outcomes

We detect chromothripsis in 40% of patients, most recurrently observed in sarcomas (35/58), germ cell tumors (2/4) and less frequently in neuroblastoma (6/29) (as seen in panel (d) of FIG. 44C). Chromothripsis frequently led to TP53 loss (10/29), amplification of MYC, VEGFA, and MDM2. Additionally, in 2 patients chromothripsis resulted in oncogenic fusions (MAN1A2-ACBD6 and PAX3-FOXO3) (as seen in panel (e) of FIG. 44C). Previous studies have proposed an association between whole genome duplication (WGD) and poor outcomes in cancer. WGD were seen in 42/114 patients with an enrichment in sarcoma (24/54), carcinomas (3/7), and neuroblastoma (11/30) (as seen in panel (f) of FIG. 44C).

### xiii. Biological and Clinical Implications of Tumor Mutation Burden Across Variant Classes

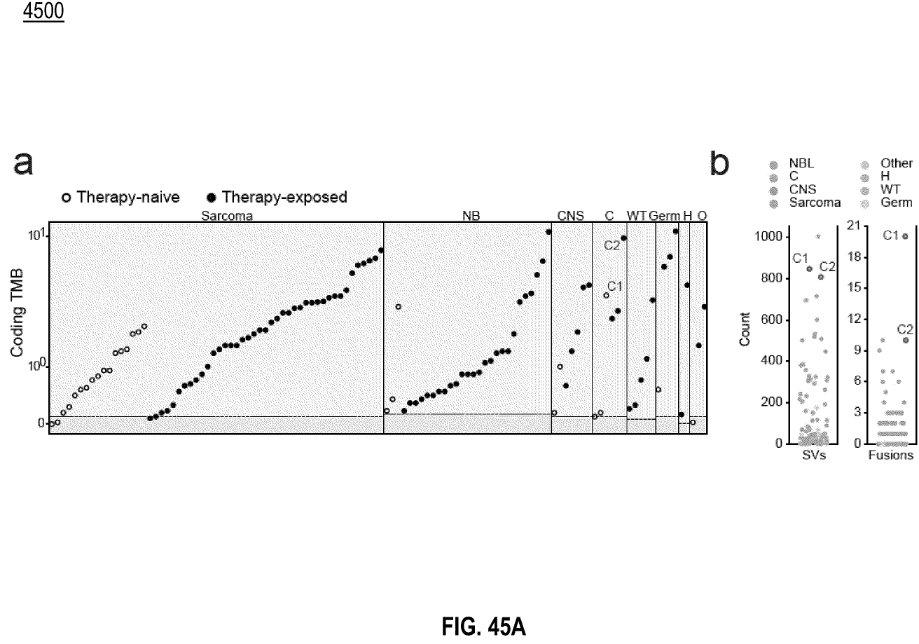

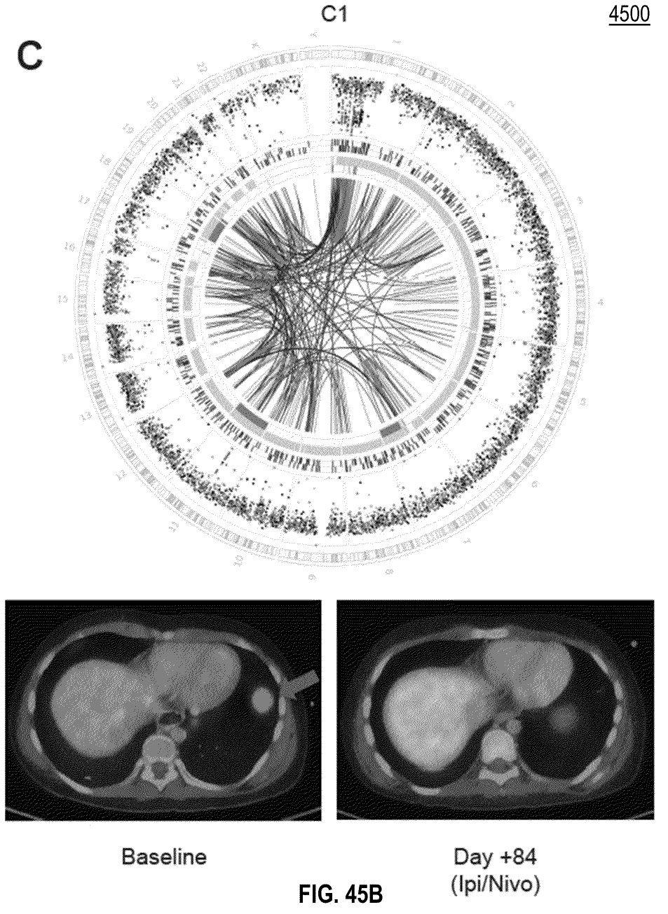

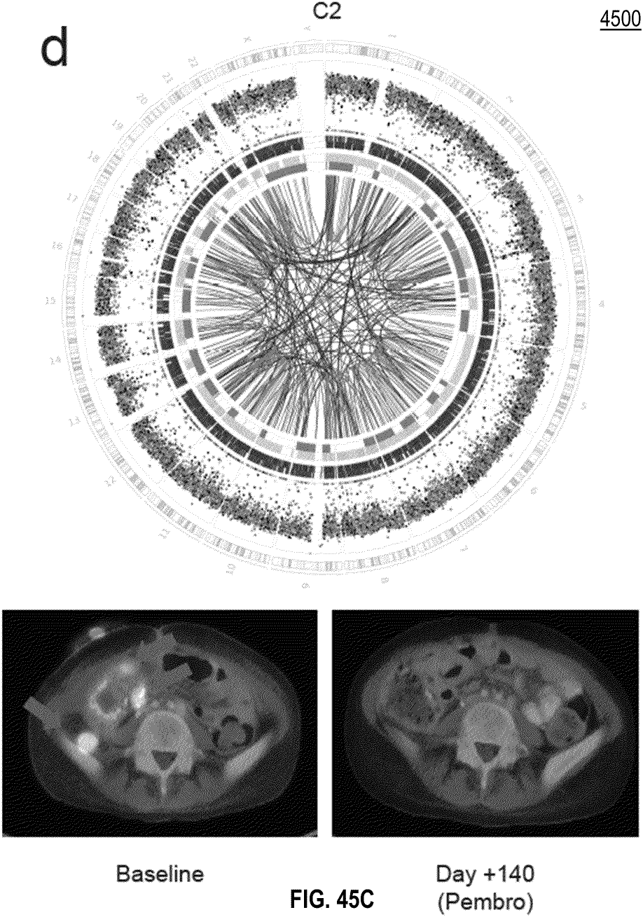

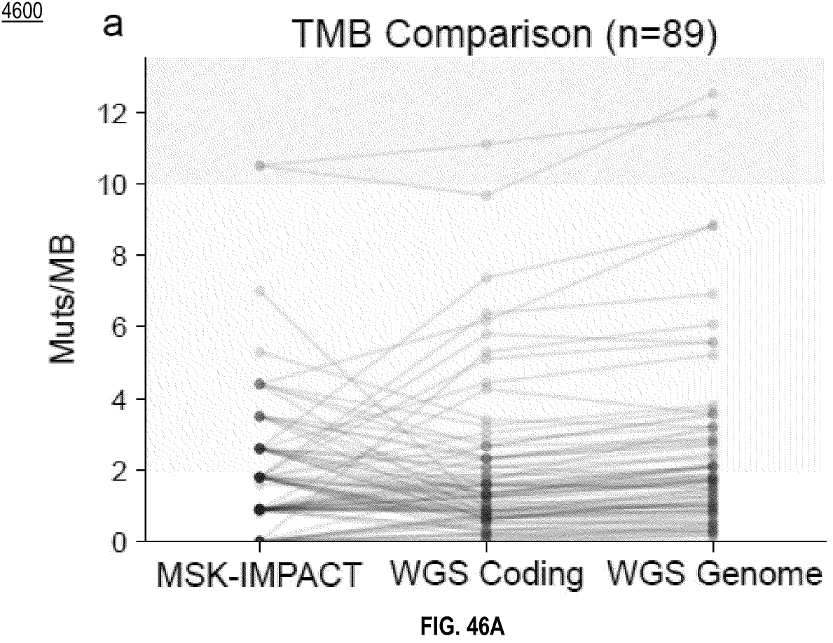

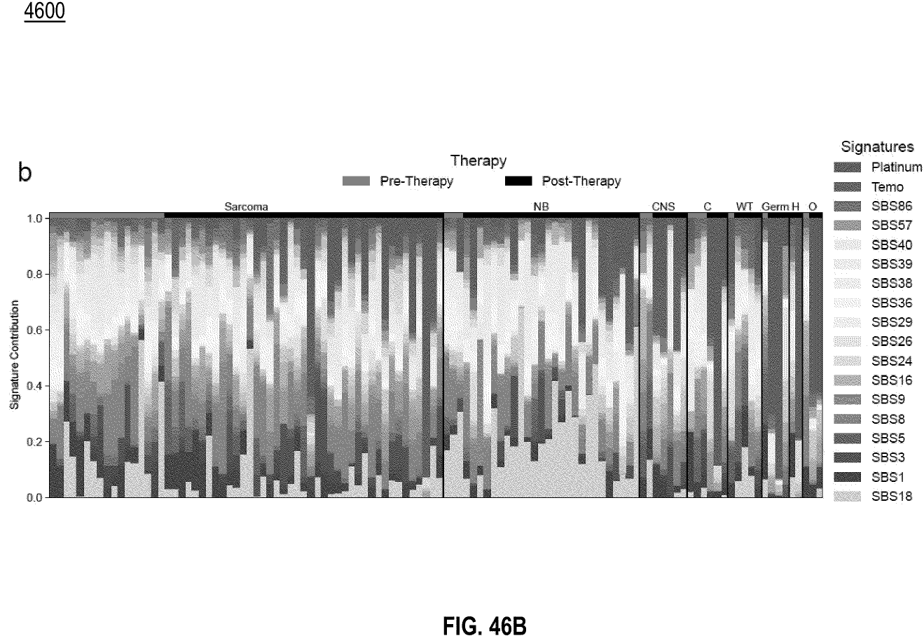

Panel based approaches derive estimates of TMB, MSI scores and mutation signatures whereas WGS directly quantifies genome wide mutation burden across all variant classes (as shown in FIG. 45A and FIG. 46A). We observed higher overall (8.3-fold) TMB estimates in our cohort, relative to reports in pediatric cancer (as shown in panel (a) of FIG. 45A). TMB was higher in therapy exposed compared to treatment-naive patient samples (0.1-11.2 in treated vs 0-2.7 treatment-naive, Mann-Whitney test, p=1.892e-04) and correlated with evidence of treatment related signatures (e.g., temozolomide, platinum) (as shown in FIG. 46B), observed in 45/114 patients pointing to persistence of clones that were exposed to and survived cancer therapy.

Patients H135022 (adrenocortical carcinoma) and H135462 (clear cell carcinoma) had progressive on-treatment metastatic disease and in the absence of therapy informing biomarkers by clinical testing were at the end of their therapeutic options. cWGTS analyses revealed a profoundly rearranged genome scoring these two patients as the highest in fusion burden and SV burden in the cohort (as shown in panel (b) of FIG. 45A and FIGS. 45B-C). H135022 was treated with checkpoint blockade (nivolumab/ipilimumab), resulting in complete response after three cycles of therapy and is disease-free 26 months after therapy cessation (as shown in FIG. 45B) whereas patient H135462 was treated with pembrolizumab, achieved a complete response after 6 cycles, and remains disease-free 10 months after therapy.

These findings demonstrate the value of cWGTS to fully assess the level of genomic instability across variant classes and highlight the need to further evaluate SV and fusion gene burden as biomarkers of response to immune checkpoint blockade therapies.

### xiv. Derivation of Comprehensive WGS Profiling in Cell-Free DNA

Our study evaluated key technical considerations of cWGTS in FF biopsies, as an optimal source of tumor DNA. However, limited biopsies may restrict access to cWGTS for all patients. Cell-free DNA (cfDNA) from blood plasma represents an alternative source of tumor cell DNA for profiling at diagnosis. Recently cfDNA WES profiling approaches have shown promise in the setting of high burden metastatic disease. However, the potential of high-depth WGS as a means to comprehensively assess (SNVs, indels, CNVs, SVs) a patients cancer genome from cfDNA has been largely unexplored.

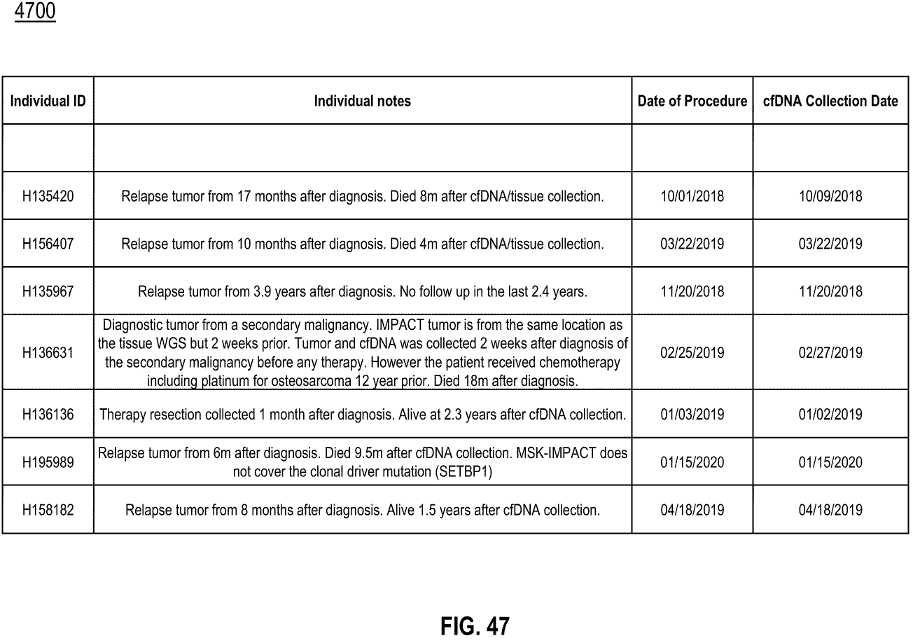

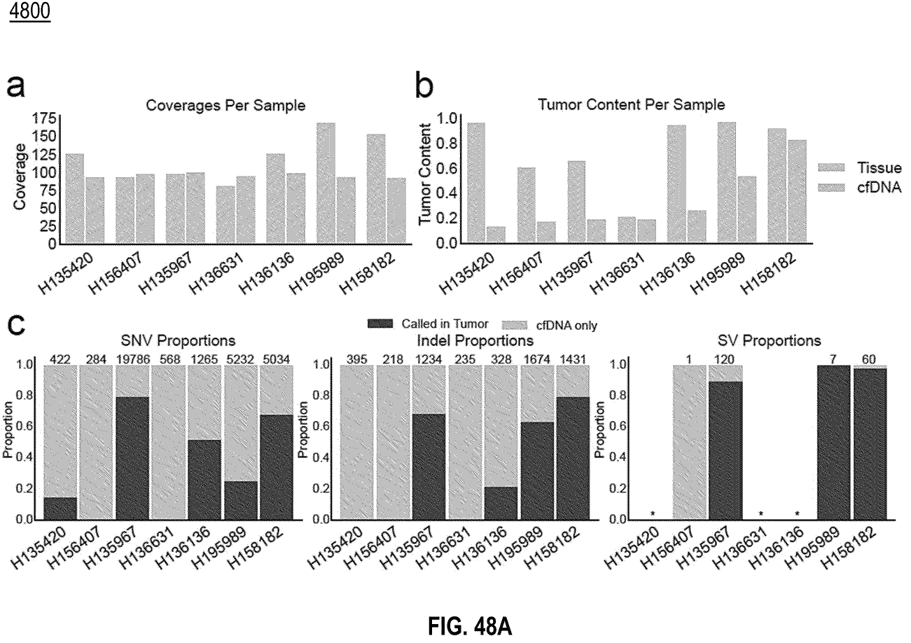

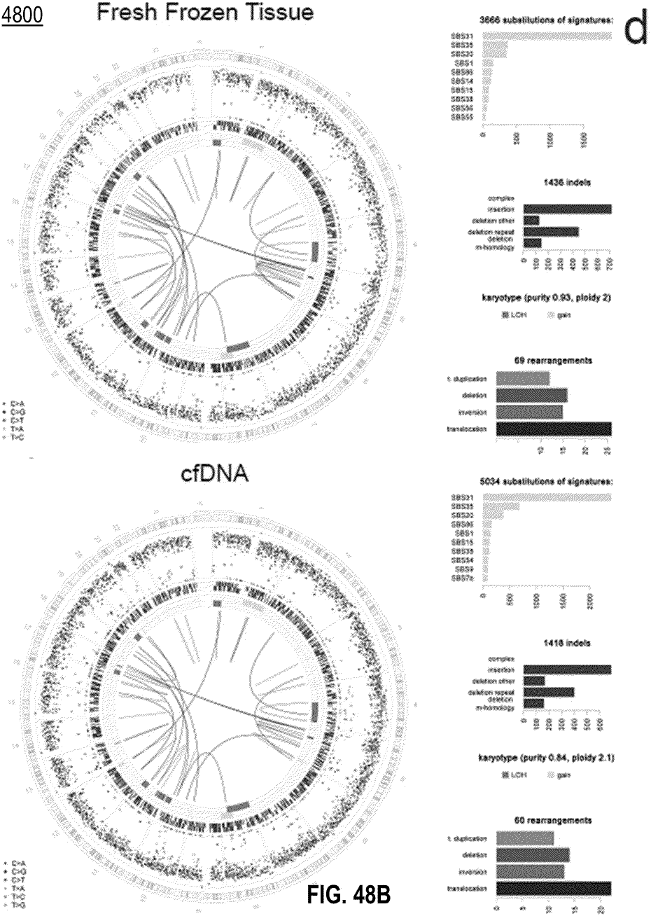

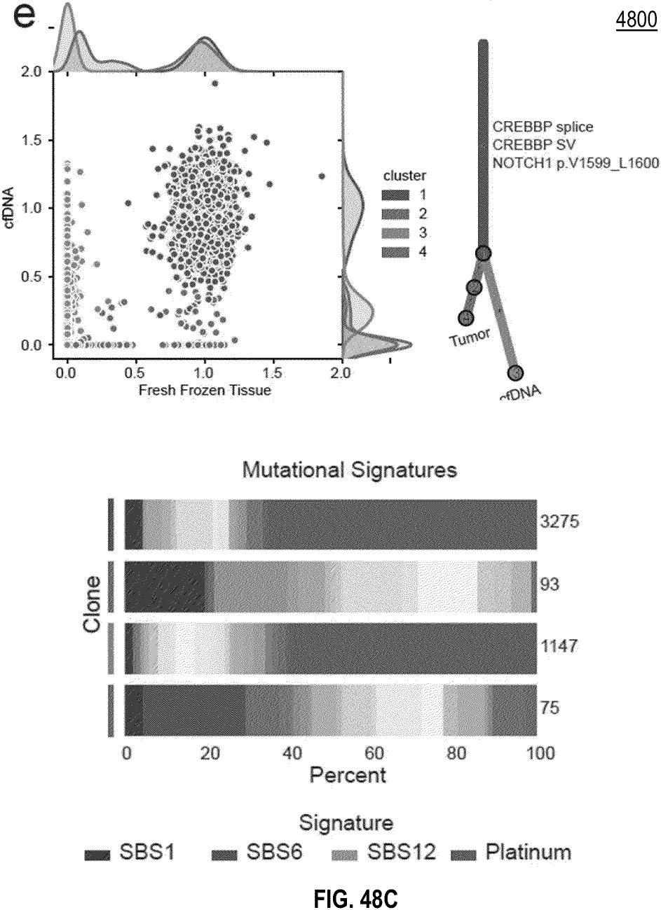

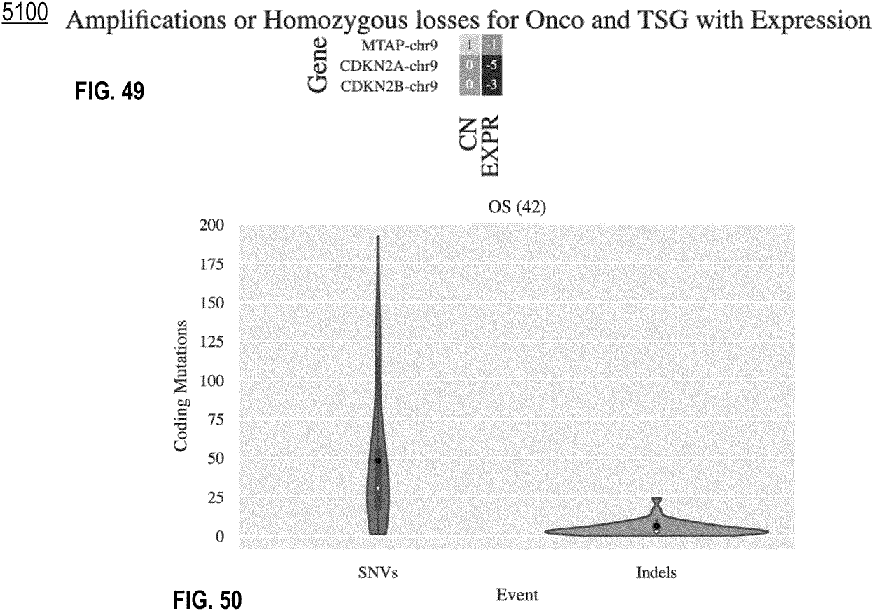

In an exploratory analysis, we performed WGS from matched FF samples and cfDNA from seven patients collected tumor biopsy (at least a portion of which is shown in table 4700 of FIG. 47). cfDNA genome wide coverage ranged from 94-102x (as seen in panel (a) of FIG. 48A) with a wide range of tumor content in cfDNA ~10-83% (as seen in panel (b) of FIG. 48A). To assess the suitability of cfDNA for unbiased genome-wide mutation detection, we performed de novo mutation calling in cfDNA and compared results to FF analyses.

WGS from cfDNA did not present technical limitations in data generation or false positive variant calling across mutation classes. Derivation of high-quality variant calls was contingent upon the quantity of circulating tumor DNA (ctDNA). In four patients with ctDNA content sufficient for CNA detection, we establish good concordance between the FF and cfDNA CNA profiles (as seen in panel (c) of FIG. 48A). Strikingly, for patients with high ctDNA content (e.g., IH158182) we derived a near complete picture of the genome-wide mutation patterns demonstrating that cancer genomic landscape can be fully recapitulated by cfDNA WGS (as shown in FIG. 48B). Importantly, for patient H135967 we showcase that even with an estimated ctDNA content of 20% the same threshold used for analyses of FF material, we can detect all the known oncogenic events in the FF sample across variant classes, which include a TP53 substitution, MYC and CCNE1 amplifications, and SVs targeting ARID1A and ATRX.

We further demonstrate the potential of cfDNA to capture a better representation of variants across the tumor phylogeny compared to solid biopsies (as shown in table 4700 of FIG. 47) and detected cfNDA specific subclones (as shown in FIG. 48C). These results provide the first proof of concept for the feasibility of deriving tumor WGS data from a liquid biopsy.

### xv. Discussion

We present a comprehensive technical assessment for cWGTS implementation in clinical practice. We demonstrate that using a single integrated workflow, cWGTS captures the full spectrum of cancer-associated genomic alterations that are assessed using a diversity of standard of care diagnostic assays. With implementation of best laboratory and computational practices we execute an end-to-end sample-to-report turnaround time within 9 days, which is aligned to clinical needs for diagnosis and care decisions. Despite 5-10 fold lower sequencing coverage, we demonstrate that in matched biopsies, cWGTS recovered all clinically reported variants by high-depth targeted profiling assays. We establish >80x coverage and tumor purity of at least 20% to attain this sensitivity. However, this sets a stringent quality threshold on fresh frozen tumor specimens which are not as broadly available as FFPE. However, a major limitation of WGS in FFPE is a high error rate in genome wide calls. To this end, we provide feasibility data demonstrating that WGS profiling can be leveraged in patients with limited surgical biopsies, but who have high cfDNA content in circulation. Future optimizations on fresh frozen biopsy preparation and cfDNA variant calling methodologies will enable expansion of WGS approaches in clinical oncology.

To support cWGTS variant annotation and prioritization, we implemented an analytical workflow that learns from variant annotation databases and integrates signals from germline mutations, somatic DNA and RNA-seq findings. This allows us to validate and prioritize novel and non-coding SVs. Consistent with recent literature for pediatric and rare cancers, >50% of patients had additional findings of established biological or clinical significance. The majority of these findings were mutations in rare cancer genes (germline and somatic), SVs, fusion genes and genome wide mutation signatures that targeted panels are not optimally designed to identify. Importantly, we demonstrate that only a minority of such additional findings would be captured by WES and RNAseq.

The clinical relevance of cWGTS extends beyond that of rare cancers. We show that by cWGTS we detect the full spectrum of cancer associated mutations in 99% of patients. The vision of patient tailored medicine warrants the delivery of clinical decisions that extend beyond a single druggable biomarker and rather consider the composite readouts from a patient’s cancer genome that inform on a patient’s a priori risk of developing cancer, diagnosis, likelihood of treatment response, risk of progression and therapeutic vulnerabilities. With increasing implementation of cWGTS on well-annotated clinical specimens our ability to interpret cWGTS findings will improve and by extension the clinical utility of cWGTS will expand. As the economic barriers to cWGTS are mitigated, in time a single comprehensive assessment of the cancer genome is positioned to replace multiple targeted diagnostic tests in prospective clinical sequencing.

### Online Methods

Another cWGTS analysis can be performed on tumor samples and healthy samples according to the systems and methods described herein.

### i. Study Participants

Patients who were seen within the Department of Pediatrics at Memorial Sloan Kettering Cancer Center with presumed or established solid tumor malignancies (including CNS tumors) were eligible to enroll onto the study/analysis. All participants were enrolled on an institutional prospective tumor/germline sequencing protocol (ClinicalTrials.gov number, NCT01775072) with informed consent. Patients with newly diagnosed as well as relapsed/refractory disease were eligible. Adults with pediatric-type malignancies or rare cancers up to the age of 39 were also eligible to enroll.

### ii. Clinical Profiling

DNA extracted from formalin fixed paraffin embedded (FFPE) tumor and blood samples (as a matched normal) were sequenced using MSK-IMPACT, an FDA-approved and New York State Department of Health validated panel used to sequence patients’ tumors at MSKCC. MSK-IMPACT captures protein-coding exons of 468 cancer-associated genes, introns of frequently rearranged genes and genome-wide copy number probes. Tumor samples were sequenced at 800x average depth of coverage, whereas peripheral blood samples at 600x. Established pipelines followed by manual review were used to characterize germline and acquired somatic mutations, copy number variants (CNVs) and if targeted, genomic rearrangements as previously described. Germline data for alterations in cancer predisposition genes were analyzed in 88 genes as previously described. For select tumor indications (i.e. sarcomas) MSK-Fusion, a New York State approved RNA-capture assay that targets common RNA fusion genes in solid tumors was also performed. Clinically relevant finding were annotated using OncoKb tiers 1-4.

### iii. Research Sequencing Approaches

### DNA Extraction

For 114 subjects enrolled in the study, tumor DNA was extracted from fresh frozen or OCT tissue biopsies and matched normal DNA from buffy coat using the DNeasy Blood & Tissue Kit (Qaigen catalog # 69504) according to the manufacturer’s protocol with incubation at 55° C. for digestion. FFPE tissue was deparaffinized using heat treatment (90° C. for 10′ in 480 µL PBS and 20 µL 10% Tween 20), centrifugation (10,000×g for 15′) and ice chill. Paraffin and supernatant were removed, and the pellet was washed with 1 mL of 100% EtOH followed by an incubation overnight in 400 µl of 1 M NaSCN for rehydration and impurity removal. Tissues were subsequently digested with 40 µl of Proteinase K (600 mAU/ml) in 360 µl Buffer ATL at 55° C. DNA isolation proceeded with the DNeasy Blood & Tissue Kit (QIAGEN catalog # 69504) according to the manufacturer’s protocol modified by replacing AW2 buffer with 80% ethanol. All DNA was eluted in 0.5X Buffer AE.

### Whole Genome Sequencing

After PicoGreen quantification and quality control by Agilent BioAnalyzer, 500 ng of genomic DNA were sheared using a LE220-plus Focused-ultrasonicator (Covaris catalog # 500569) and sequencing libraries were prepared using the KAPA Hyper Prep Kit (Kapa Biosystems KK8504) with modifications. Briefly, libraries were subjected to a 0.5X size select using aMPure XP beads (Beckman Coulter catalog # A63882) after post-ligation cleanup. Libraries were not amplified by PCR and were pooled equivolume for sequencing. Samples were run on a NovaSeq 6000 in a 150 bp/150 bp paired end run, using the NovaSeq 6000 SP, S1, S2, or S4 Reagent Kit (300 Cycles) (Illumina). Tumors were covered to an average of 95X (range 67-181) and normals at 50X (range 32-159).

### RNA Extraction

Tumor tissue from fresh frozen biopsies were homogenized in 1 mL TRIzol Reagent (ThermoFisher catalog # 15596018) followed by phase separation with 200 µL chloroform. RNA was extracted from the aqueous phase using the miRNeasy Micro Kit (Qiagen catalog # 217084) on the QIAcube Connect (Qiagen) according to the manufacturer’s protocol with 350 µL input. Samples were eluted in 15 µL RNase-free water

### Whole Transcriptome RNA Sequencing

After RiboGreen quantification and quality control by Agilent BioAnalyzer, 18 ng-1 µg of total RNA with an RNA integrity number varying from 1 to 9.9 underwent ribosomal depletion and library preparation using the TruSeq Stranded Total RNA LT Kit (Illumina catalog # RS-122-1202) according to instructions provided by the manufacturer with 8 cycles of PCR. Samples were barcoded and run on a HiSeq 2500 in Rapid Mode or HiSeq 4000 at PE100 or on a NovaSeq 6000 at PE150, using the HiSeq Rapid SBS Kit v2, HiSeq 3000/4000 SBS Kit, or NovaSeq 6000 SP, S1, S2, or S4 Reagent Kit (300 Cycles) (Illumina). Sequencing was performed to achieve a median of 83 million paired reads per sample.

### cFDNA Extraction and WGS

For 7 patients with informed consent, cell-free DNA (cfDNA) was extracted from plasma using MagMAX cfDNA isolation kit. After PicoGreen quantification, 47-500 ng of cfDNA were used to make sequencing libraries using the KAPA Hyper Prep Kit (Kapa Biosystems KK8504) with 4 cycles of PCR and pooled equimolar. One sample with sufficient input was prepared PCR-free. Samples were run on a NovaSeq 6000 in a PE150 run, using the NovaSeq 6000 SBS v1 Kit and an S4 flow cell (Illumina). The average coverage per sample was 1X.

### iv. Workflow Optimization

In order to achieve stable turnaround times of 9 days, dedicated resources and optimizations were needed, such as to minimize human steps in the process. In the sequencing core, lab technicians along with sequencers were needed to process and quality control the incoming samples. A high throughput connection was used to transfer sequencing data to the bioinformatics core with automatic notifications. An ETL cron job was developed to synchronize relevant de-identified metadata regularly from clinical systems. The data and bioinformatics analyses were tracked and automated using the Isabl platform. Many of the bioinformatics algorithms were optimized for higher levels of parallelization. In order to achieve stable algorithm turnaround times, parallelization was often split by estimated amount of work (e.g., number of reads; https://github.com/papaemmelab/split_bed_by_reads) rather than genomic length. Processing was performed within a heavily shared internal high performance computing (HPC) cluster with around 4000 cores. Results were automatically curated and prioritized using both cached databases as well as live APIs in order to reduce interpretation time.

### v. Bioinformatic Analysis

Analysis of cWGTS data was executed using Isabl platform and included: 1. Data QC; 2. Ensemble variant calling for germline and somatically acquired mutations from at least two out of three algorithms run for each variant class; 3. Signature extraction (i.e. mutation signatures, microsatellite Instability score, gene expression); 4. Variant classification; and, 5. The generation of a clinical prototype summary report. Briefly, upon completion of each sequencing run, Isabl imports paired tumor-normal FASTQ files, executes alignment, quality control algorithms and generates tumor purity and ploidy estimates. For samples with sufficient coverage (>60x) and tumor purity (>20%) ensembl variant calling for each variant class (substitutions, insertions and deletions and structural variations) is performed. High confidence somatic mutations were classified with regards to their putative role in cancer pathogenesis and statistical post-processing enables the derivation of microsatellite instability scores and mutation signatures. RNA-seq data were independently analyzed for acquired fusions and gene expression metrics in a subset (n=101). For a subset of patients with consent (n=100) for germline analyses, the normal genome was also independently analyzed.

Clinical relevance of mutations in common cancer genes was annotated using OncoKb, COSMIC, Ensembl Variant Effect Predictor, VAGrENT, gnomAD and ClinVar databases. Additionally, integration of signals across data modalities (germline, somatic mutations, somatic signatures, copy number segments and gene expression profiles) was executed to further determine the significance of observed events. Population filtering, database comparison and somatic data integration were performed using methods in accordance with the American College of Medical Genetics and ClinGen Somatic/Germline Data Integration subcommittee. Last, findings were automatically embedded into a single page summary (.html) report containing high-level clinical data, quality control metrics, genetic findings and relevant data visualization plots (e.g., CIRCOS plots, mutation signatures, gene expression clustering by tSNE). Putative findings of clinical relevance identified by WGS and RNA-seq were reviewed by an interdisciplinary team of clinical oncologists, molecular pathologists and cancer genomics experts and categorized with regards to their relevance in clinical practice as 1. Diagnostic 2. Risk predisposition for germline variants, 3. Prognostic, 4. Therapy informing, 5. Pathogenic, 6. Likely Pathogenic or 7. Variant of unknown significance (VUS).

### vi. Pipeline Overview

### Whole Genome/Transcriptome Alignment and Quality Control

Whole genome paired-end reads were aligned to human reference genome (GRCh37d5) using BWA-mem (v0.7.17) as a part of the pcap-core v2.18.2 wrapper (https://github.com/cancerit/PCAP-core). The wrapper includes marking of duplicates using Picard. Whole transcriptome sequencing reads were aligned using Spliced Transcripts Alignment to a Reference, STAR (v2.5.4b, https://github.com/alexdobin/STAR) with Ensembl 75 for transcript information. Upon alignment BAM files for tumor/normal WGS and tumor RNAseq data for each individual were compared using Conpair in order to detect potential sample swaps and cross-individual contamination. Genome-wide median coverage was calculated using Mosdepth with minimum mapping quality of 20. Tumor purity and ploidy was estimated using Battenberg (https://github.com/cancerit/cgpBattenberg). For tumors without aberrant copy number segments but with somatic substitutions called, tumor purity was estimated from VAF distribution assuming a ploidy of 2 genome-wide.

### Identification of Somatic Mutations in Whole Genome Sequences

Somatic alterations were detected comparing the tumor against the matched normal for each variant type. All bioinformatic tools were launched using an in-house wrapper. Allele-specific subclonal copy number changes were detected using Battenberg (cgpBattenberg v1.4.0) (https://github.com/cancerit/cgpBattenberg). Single nucleotide variants (SNVs) were identified using Strelka2 (v2.9.1 with manta v1.3.1), (https://github.com/lllumina/strelka), MuTect2 (gatk:v4.0.1.2), (https://github.com/broadinstitute/gatk) and CaVEMan (cgpCavemanWrapper v1.7.5) (https://github.com/cancerit/cgpCaVEManWrapper). Variant post-processing was done using default flags for Strelka and MuTect2 while for CaVEMan, cgpCavemanPostprocessing (v1.5.2) was used filtering for sequencing artefacts utilizing a panel of 100 unmatched normals (https://github.com/cancerit/cgpCaVEManPostProcessing). Small insertions and deletions (indels) were detected using Strelka2, MuTect2, and Pindel (cgpPindel v1.5.4) (https://github.com/cancerit/cgpPindel) and filtered against a panel of 100 unmatched normals. Structural genomic variants (SVs) were identified using SvABA (~v1.0.0 commit 47c7a88) (https://github.com/walaj/svaba), GRIDSS (v2.2.2) (https://github.com/PapenfussLab/gridss) and BRASS (v4.0.5 with GRASS v1.1.6) (https://github.com/cancerit/BRASS) using a panel of 100 in house unmatched normals. Finally, microsatellite instability status was assessed using MSISensor (v0.5) (https://github.com/ding-lab/msisensor).

### Variant Consolidation and Annotation

VCF files for SNVs and indel were merged with an in-house wrapper using Chromosome, Position, Reference Allele, and Alternative Allele. The merged VCFs were annotated with VAGrENT(v3.3.0, https://github.com/cancerit/VAGrENT) and VEP (v92, https://github.com/Ensembl/ensembl-vep). VCF files for SVs were merged using MergeSVvcfs (v1.0.2, https://github.com/papaemmelab/mergeSVvcf). High-confidence mutations were designated as those that were passed by at least 2 callers.

### Identification of Germline Mutations

Germline single nucleotide polymorphisms (SNPs) and indels were detected using Stelka2 and Freebayes (v1.2.0, https://github.com/ekg/freebayes) with an in-house wrapper. VCF files were merged and annotated using the same procedure used for the somatic variants. Germline variants called by both callers were considered high-confidence. Germline variants were prioritized for review by filtering for recurrence in the current cohort, frequency in any population of 1000 genomes/Gnomad and ClinVar.

### Characterization of Gene Fusions

Gene fusions were identified using three different callers; FusionCatcher (v1.0.0, https://github.com/ndaniel/fusioncatcher), STAR-Fusion (v1.3.1, https://github.com/STAR-Fusion/STAR-Fusion), and FuSeq (v1.1.1, https://github.com/nghiavtr/FuSeq). Calls were merged by gene pair and annotated using FusionCatcher’s databases. Fusions were considered confident if called by at least 2 callers. Events were visualized with the plotting functionality by Arriba (https://github.com/suhrig/arriba).

### Gene Expression Analysis

Gene expression profiles were ascertained in Transcripts Per Million (TPM) using SALMON (v0.10.0, https://github.com/COMBINE-lab/salmon). tSNE was performed using python scikit-learn (v0.21.1, https://scikit-learn.org/) and visualized interactively using python bokeh (v1.2.0, https://docs.bokeh.org/).

### vii. Identification of Gene Expression Biomarkers

Expression biomarkers were assessed using the methodology outlined by Horak et al. Only actionable genes outlined in the publication supplementary as assessed by severe overexpression, overexpression, severe underexpression, or underexpression were evaluated. An internal reference cohort of 274 tumor rna samples was used as a baseline. A gene was considered over/underexpressed if expression was in the top or bottom ten percent of the reference cohort, while severe over/underexpression was categorized as expression in the top or bottom five percent.

### Identification of Mutation Signatures of Point Mutations

Mutational signature analysis was performed with the MutationalPatterns package (v1.6.1, https://bioconductor.org/packages/release/bioc/html/MutationalPatterns.html) and using the COSMIC Mutational Signatures (v3.1) with the addition of Temozolomide signature from Kucab et al.

### Assessment of ITH Between Matched MSK-IMPACT and WGS Samples

In 87 patients we investigated the possibility of ITH between the matched FFPE and fresh frozen samples that underwent MSK-IMPACT and WGS sequencing, respectively. ITH can present as discordance in mutations (substitutions/indels), CN changes and SVs. As MSK-IMPACT provides limited information on the latter two, we focused on mutations and arm-level CN changes and performed three independent analyses. First, we compared the effective coverage in WGS between the called and missed mutations which showed no systematic bias. We then assessed the clonal representation between MSK-IMPACT and WGS by performing a proportion test comparing the VAFs reported by the two assays adjusting for assay-specific local depth and purity. This analysis was confined to 221 clinically reported substitutions and indels in 468 genes included in the MSK-IMPACT irrespective of their oncogenic status. Mutations with a p-value <0.05 have a statistically significant difference in clonal presentation suggesting ITH. Second, we compared the genome-wide CN changes in both assays. CN profiles from MSK-IMPACT generated using FACETS were available for 74 tumors and were compared to the Battenberg output from WGS. Third, in 25 patients where DNA was available, we performed re-sequencing of 44 discordant mutations using the MSK-IMPACT panel on the same DNA that underwent WGS sequencing at a median depth of 438X. We checked the tumor purity of both MSK-IMPACT assays to mitigate the effects of technical issues on mutation calling.

### Inference of Clonal Structure

Clonal structure was analyzed using high confidence SNVs called in each biopsy or the union of SNVs whenever multiple biopsies were available for a patient. DPClust (v0.2.2, https://github.com/Wedge-Oxford/dpclust) was used for calculation of cancer cell fraction corrected for purity and local copy number as well as clustering and assignment of mutations across samples with the exception of the Gibbs Sampling Dirichlet Process step which has been rewritten internally for performance. Clonal ordering was deduced using clonevol (v0.99.11, https://github.com/hdng/clonevol). Mutational signatures were computed in each cluster independently (as described above). Figures were generated with python matplotlib (v3.1.0, https://matplotlib.org/). All steps are run using an inhouse wrapper.

### Estimates of Telomere Length

Estimation of telomere length/content is computed from tumor and normal bam files using Telseq (v0.0.2, https://github.com/zd1/telseq). Telomere length/content is compared between tumor and normal to deduce lengthening or shortening in the tumor with respect to the normal.

### Derivation of Subsampled BAM Files and Sensitivity Assessment

A total of 298 subsampled BAMs were generated using samtools (v1.11, https://github.com/samtools/samtools) view command with the subsampling option. Median coverages were calculated for the original BAMs using Mosdepth (v0.2.5, https://github.com/brentp/mosdepth) with mapping quality > 20 and then used to calculate fractions to down-sample to approximately 100x, 80x, 60x, and 30x-40x where original coverage allowed. Mosdepth was then used again to verify that the median coverage of the subsampled BAM fell within +/- 5x of the desired coverage. De novo variant calling and annotation was then performed independently on the subsampled BAM files as described previously.

### cFDNA Tumor Content and Variant Comparison

Tumor content in cfDNA specimens was estimated using Battenberg and manual inspection with the help of SNV VAF density plots. This was compared between cfDNA specimen and tissue specimen from the same individual. De novo variant calling was performed independently using the methods described for identification of somatic mutations in WGS. SNVs and indels were compared using Chromosome, Position, Reference Allele, and Alternative allele. SVs were compared using MergeSVvcfs (v1.0.2, https://github.com/papaemmelab/mergeSVvcf) and copy number alterations by using the segmentation outputs from Battenberg. Further analysis was done to compare specific clinical variants identified by MSK-IMPACT using hileup (v1.0.0, https://github.com/brentp/hileup) to pile up all unique reads aligning to a genomic position and investigate support for the reference and alternative alleles. Clonal structure across tissue and cfDNA samples was inferred using the same methods as described before followed by analysis of clone-specific mutational signatures. All mutations across the clonal structures were then piled-up across corresponding fresh frozen WGS data to look for evidence of mutations in both specimens.

### Variant Curation and Characterization of Incremental Findings from cWGTS

Variant curation of targeted NGS assays were performed as previously described. To assess variants identified by cWGTS, a multidisciplinary team comprised of disease experts, clinical geneticists, molecular pathologists, and genomics experts assembled regularly to classify molecular alterations (somatic and germline), mutation signatures, and gene expression data. Incremental findings of cWGTS were defined as established oncogenic alterations or signatures not identified by matched MSK-IMPACT somatic or germline NGS (DNA) or ArcherDx targeted NGS (RNA). Incremental findings were further classified as clinically relevant if they met one of the following criteria: 1) diagnostic finding, 2) established prognostic finding, 3) likely pathogenic or known pathogenic germline predisposition event, 4) treatment informing finding, or 5) novel driver oncogenic fusion.

### APPENDIX

1. Glossary

| | |
|---|---|
| Term | Definition |
| BAF | B allele frequency |
| Breakpoint | Genomic coordinates in which a rearrangement of the genome has occurred |
| CCF | Cancer cell fraction. Prevalence of an event in cancer cells. A value near 1, represents an event in every cancer cell. |
| Clonal | Present in all cancer cells. |
| Hotspot | Localized areas of the genome with high probability to be mutated in cancer. |
| logR | Log transformed ratio (e.g., normalized measure of differences in coverage). |
| MAF | Minor allele frequency. |
| Mapping Quality | Score representing the confidence that a read is correctly mapped in the genome. |
| PON | Panel of normal. Reference data from normal samples generally used for filtering sequencing artifacts and common SNPS. |
| Purity | Percentage of malignant cells in tumor sample. |
| Repeat-masker | Repeated or low complex areas of the genome. |
| SNP | Single nucleotide polymorphisms. Generally refer to general population variation. |
| Subclonal | Present in a subset of cancer cells. |
| TPM | Transcripts per million. A metric of RNA expression. |
| TSG | Tumor suppressor gene |
| TSNE | A t-distributed stochastic neighbor embedding. A dimension reduction technique used to visualize high-dimensional data. |
| VUS | Variant of unknown significance. Event for which we do not know if it causes a pathogenic effect. |

### 2. FusionCatcher Databases

The author of FusionCatcher has compiled sets of fusions, artifacts, and gene-level annotations for use as reference annotation. The sets of fusions, artifacts, and gene-level annotations are describe in detail here: https://github.com/ndaniel/fusioncatcher/blob/master/doc/manual.md#62---output-data-output-data.

### 3. Feather File Format

A binary file type created by Apache for storing datatables. The on-disk format is designed to be very fast for reading tables into memory. See https://arrow.apache.org/docs/python/feather.html.

### 4. Known Cancer Genes

Oncogenes and tumor suppressor genes are compiled from OncoKb (https://www.oncokb.org/cancerGenes) and Cancer Gene Census (https://cancer.sanger.ac.uk/census). Additional research genes are added as needed (e.g. DLG2 TSG).

### 5. Known or Predicted Hereditary Cancer Genes

Gene sets from multiple publications are combined to create a broad list of suspected hereditary cancer genes. The broad list includes

https://cdn.jamanetwork.com/ama/content_public/journal/oncology/938471/coi200005supp1_prod.pdf?Expires=2147483647&Signature= QfgUCBRlr0dgTlW9~13LEnCHnHPhvPZiP48OfwNPoA7tmcUfcjBX56QyjSwqwD2d-YJ0ei5ryBkGPaB0TzJO8M8g4pZUNhhYeduiJyJxOAhfk9oqe9jZ4Jzkk~PccB6BSCqndhH0obxnXQg71un-SXsMWanHvzbjhS41hNmsaWw~rxeqj2ffKVzsgFudCHRRnpQla7f26pc~Y~Hdl4xmrTW95bl1pADgPdGFLLDrt0t3rQy58dboMlz2egpDFJ hYZpg~Np1Ri58bpVMgvFW5LCUuT4fsUM7haaJ-l770S5dc7Uh4~U2T7oZAQdtSW-Z96mew36RTCwgL3bxjzsGduA_&Key-Pair-Id=APKAIE5G5CRDK6RD3PGA, https://ascopubs.org/doi/10.1200/JCO.19.02760, HGMD, and ClinVar. Additional research genes are also added as needed.

### 6. Predicted Haploinsufficient Genes

Haploinsufficient genes are genes which cannot maintain normal function with just a single wildtype copy. The HI score from DECIPHER (https://www.deciphergenomics.org/) is used to determine if a gene is likely haploinsufficient. The threshold used is <10% score for predicted haploinsufficient.

Overview of Various Embodiments: The widespread adoption of high throughput technologies has democratized data generation. However, data processing in accordance with best practices remains challenging and the data capital often becomes siloed. This presents an opportunity to consolidate data assets into digital biobanks-ecosystems of readily accessible, structured, and annotated datasets that can be dynamically queried and analysed. Various embodiments provide a customizable plug-and-play platform for the processing of multimodal patient-centric data. In one embodiment, the disclosed architecture includes a relational database (DB), a command line client (CLI), a RESTful API (API) and a frontend web application (Web). The system supports automated deployment of user-validated pipelines across the entire data capital. A full audit trail is maintained to secure data provenance, governance and ensuring reproducibility of findings. As a digital biobank, the system supports continuous data utilization and automated meta analyses at scale, and serves as a catalyst for research innovation, new discoveries, and clinical translation.

In various embodiments, novel biological and clinical insights are derived based on large and statistically powered datasets, rich metadata annotation (clinical, demographic, treatment, outcome) as well as integration of diverse data modalities generated across samples and patients (i.e. genomic, imaging). Implementation of frameworks that operate in accordance with data processing best practices is important to secure governance and provenance of digital assets, ensure quality control, and deliver reproducible findings. Analysis Information Management Systems (AIMS) for Next Generation Sequencing (NGS) data represent integrative software solutions to support the lifecycle of genomics projects. Embodiments of the disclosed approach incorporate multimodal data types in a patient or individual centric architecture.

In various embodiments, a plug-and-play platform processes individual-centric multimodal data. The system is designed to support: (1) management of data assets according to the FAIR principles (Findable, Interoperable, Accessible, Reusable), (2) automated deployment of data processing applications following the DATA reproducibility checklist (Documentation, Automation, Traceability, and Autonomy); and, (3) advanced integrations with institutional information systems across diverse data types (i.e. clinical and biospecimen databases). To support flexible workflows the system is built upon a customizable framework, that enables end-users to specify metadata and pipeline implementation. In addition, a pipeline development methodology that is guided by the principles of containerization, continuous integration, version control, and the separation of analysis and execution logic is provided. A framework for the development of digital biobanks is provided-patient-centric ecosystems of structured, annotated, and linked data that is readily computable upon, mined, and visualized.

In certain embodiments, the system may comprise four main microservices: (1) DB, an individual-centric database designed to track patients, samples, data, and results; (2) API, a RESTful API used to support authentication, interoperability, and integration with data processing environments and enterprise systems (e.g. clinical databases, visualization platforms; FAIR A1); (3) CLI, a Command Line Interface for managing and processing digital assets in a scalable data lake (i.e., genomic, imaging); and (4) Web, a frontend single page web application for data interrogation. These are part of a patient centric relational model for the integration of multimodal data types (i.e., genomic, imaging) and their corresponding relationships (individual, sample, aliquot, experiment, analyses). Web facilitates visualization of results and metadata in a single page application, API powers the linkage to other institutional information systems and is agnostic to data storage technologies and computing environments, ensuring metadata is accessible even when the data is no longer available (FAIR A2), CLI is a Command Line Client used to process and manage digital assets across computing paradigms (i.e. cloud, cluster).

In certain embodiments, DB maps workflows for data provenance, processing, and governance. Metadata may be captured across the following 5 thematic categories: (1) patient attributes; (2) samples, as biological material collected at a given time; (3) data properties including experimental technique, platform technology, and related parameters; (4) analytical workflows to account for a complete audit trail of versioned algorithms, related execution parameters, reference files, status tracking, and results deposition; (5) data governance information across projects and stakeholders (FAIR F2). All database records may be assigned a globally unique and persistent identifier (UUID; FAIR F1), whilst individuals, samples, and experiments are further annotated with a customizable human friendly identifier. All metadata stored in DB is version controlled, all changes are recorded and previous states can be recovered. Management of phenotypic data such as disease ontology can be facilitated in three ways. Firstly, the disease schema can be customized with additional fields in agreement to end-user requirements. Secondly, ontologies from established databases such as OncoTree, (http://oncotree.mskcc.org) can be integrated (i.e. https://docs.isabl.io/data-model#sync-diseases-with-onco-tree). Lastly, proprietary schemas from institutional databases (i.e. ontologies implemented in local electronic medical records) can also be incorporated, thus allowing for direct linkage between results and related metadata at an institutional level. The system’s relational model maps workflows for data provenance (e.g. Individuals, Samples, Experiments), processing (e.g. Applications, Analyses), and governance (e.g. Projects, Users). An individual-centric model facilitates the tracking of analyses conducted on experimental data obtained from related samples. Analyses are results of analytical workflows, or applications. Experiments are analyzed together and grouped in projects. Additionally, schemas to track metadata for diseases, experimental techniques, data generation platforms, and analyses cohorts are also provided. Lines with one circle represent foreing keys, whilst lines with two circles represent many to many relationships.

Life cycle of bioinformatic operations of various embodiments: system operations may be organized in a three step process: (1) project initiation and metadata registration; (2) automated data import and processing; and, (3) results retrieval for analyses. At project initiation, users specify a title, study description, and define stakeholders using Web. Individuals, samples and related experiments are registered through web forms, Excel batch submissions, or automated HTTP requests. Validation rules are enforced to ensure content quality, while account permissions and user roles guide data governance (project creation, edit, and data queries; see https://docs.isabl.io/production-deployment#multiuser-setup). To prevent dangling information, records can’t be deleted if they are associated with other instances (e.g. a sample can’t be removed if it has linked experiments). Furthermore, all database schemas can be extended with custom fields in order to address end-user metadata requirements. Once information is registered, users can interrogate the entire digital real estate using Web. A single page portal is populated with interactive panels that become available as new information is requested. Tables directly wired to API, provide searching, filtering, and ordering capabilities across different schemas and are available throughout the application (FAIR F4). Web is a Single Page Application (SPA) organized in interactive panels. Example of sample level metadata, to include sample ID, corresponding individual ID, experimental ID, species, gender, center, data generating platform, experimental technique, disease state at the time of sampling, institutional database integrations (i.e. RedCap) and version of corresponding data genome assembly. Metadata fields are flexible and customizable. Users can dynamically explore metadata by clicking the different nodes (i.e. from samples, to experiments, to all available analyses under any node). The Analysis Panel indicates execution status, version, run time, storage usage, linked experiments and offers quick access to a selected set of results (e.g. BAM files with https://github.com/igvteam/igv.js, images, log files, tables). Detail views are retrieved by clicking on any hyper-linked identifier within these tables. The project detail panel caters a birds-eye view across all analyses and experiments pertaining to a study. Similarly, the samples view provides an interactive, patient-centric, tree visualization that enables instant access to all assets generated on a given individual. Dashboards to explore metadata and access results are also provided.

In various embodiments, after metadata registration, the next step for a project is data import. CLI explores data deposition directories (i.e. sequencing core, data drives) identifying multimodal digital assets (i.e. genomic, imaging) relating to specific experiments and imports them into a scalable data directory (move or symlink). This process ensures that the link between data and metadata is stored in DB. Upon import, access permissions are configured and data related attributes are stored in the database (e.g. checksums, usage, location). Import status is updated in DB and displayed in Web. In addition to data imported for analyses, CLI also supports the registration of auxiliary assets such as an assembly reference genomes, techniques reference data (e.g. BED files), and post-processing files (i.e. data relating to control cohorts). To secure data integrity, import operations and data ownership are limited to a single admin user (e.g. a shared Linux account managed by Isabl administrators). Importantly, import logic for data and auxiliary files is entirely customizable and can be tailored to end-user requirements (i.e. cloud storage).

In various embodiments, CLI may operate on local file systems using traditional unix commands such as mv, In, cp, and rsync. Nevertheless, the data lake can be stored in cloud buckets like Amazon S3, Google Storage Buckets, or Azure Blobs. Mechanisms to push and pull data to the cloud may be implemented by the user, although there are automated solutions such as Amazon FSx for Lustre. When data is stored in the cloud, Web can be configured to retrieve and display results from these providers. Importantly, the system can compute on data located in a local file system, cloud based solutions or hybrid (local and cloud).

With respect to deploying data processing tools at scale with applications, in various embodiments, the system is a horizontally integrated digital biobank onto which existing or bespoke analytical applications can be docked and integrated in a way that confers sample-centric traceability to the analytical results. Upon data import, system applications enable standardized deployment of data processing pipelines with a Software Development Kit (SDK). Guided by experimental metadata in DB, applications construct, validate, and deploy execution commands into a compute environment of choice (e.g. local, cluster, cloud). System applications may be defined using python classes. System applications enable systematic processing of experimental data. Guided by metadata, system applications construct, validate, and deploy computing commands across experiments. Applications differ from Workflow Management Systems in that they don’t execute the analytical logic but construct and submit a command. Isabl applications can be assembly aware, this means that they can be versioned not only as a function of their name, but also as a function of the genome assembly they are configured for. This is important because NGS results are comparable when produced with the same genome version. The unique combination of targets and references, such as tumor-normal pairs, results in analyses. System applications may be based on different experimental designs, such as paired analyses, multi-targets, single-target, etc. Importantly, applications are agnostic to the underlying tool or pipeline being executed.

For example, in various embodiments, variant calling applications will tailor execution parameters and reference datasets given the nature of the data (i.e. targeted gene sequencing, whole genome sequencing, etc.). Application results are stored as analyses. Each analysis is linked to results files and specific execution parameters. Analyses can compute on data for one or more targets and references experiments (e.g. single-target, tumor-normal pairs, target vs. pool of normals, etc.). Furthermore, analyses can also track numeric, Boolean, and text results using a PostgreSQL JSON Field. To warrant a full audit trail of results provenance and foster reproducibility, the system stores all analyses configurations (parameters, reference datasets, tool versions, etc.).

In various embodiments, upon completion of an analytical workflow, ownership of output files is automatically transferred to the admin user and write permissions are removed. Once implemented, applications can be deployed system wide, on an entire project, or any subset of experiments in the database. A user-defined selection of results can be accessed through Web, which also indicates execution status, version, run time, storage usage, and linked experiments. If an analysis has already been executed, the system could prevent it’s resubmission to minimize computing usage and prevent duplication.

Operational automations: in various embodiments, to automate downstream analyses system applications define logic to combine results at a project or individual level. For example, quality control reports, variant calls, or any other kind of result are merged within a single report (for each result type). The merge operation, at the project or individual level, is triggered automatically and runs only when required (i.e. not executed if other to-be-merged analyses are ongoing). Aggregated outputs are dynamically updated as new experiments are processed by the application. All auto-merge analyses are versioned and stored in DB. CLI can facilitate automations using signals, python functions triggered on status changes to execute subsequent tasks. For instance, a signal can be configured to deploy quality control applications upon data import. At QC success, another signal could deploy a complete suite of applications tailored to the nature of the experimental data. In case of automation failure, the system will send notifications to engineers (e.g., via email), with error logs and instructions on how to restart the automation. API may be equipped with an asynchronous tasks functionality useful to schedule backend work. For example, a task can be configured to sync metadata from institutional systems every 2 hours.

In various embodiments, users can retrieve results using three main mechanisms: (1) visualization through Web; (2) programmatic data access with CLI; and, (3) direct data lake access. For each analysis, job execution status (i.e. pending, in progress, complete), as well as a defined list of results can be directly accessed through Web (with support for strings, numbers, text files, images, PDF, BAM, FASTA, VCF, PNG, HTML, amongst others). Web access to NGS data is further enabled using IGV.js. Additionally, CLI represents a programmatic means of entry to the entire data capital. A suite of command line utilities for metadata, data, and results retrieval is readily available. For example, queries can be constructed to identify samples of interest matching a range of attributes (i.e. patients, samples, analyses metadata) and retrieve specified results files (e.g. VCF files).

In various embodiments, the codebase powering the client can be imported as a python package fostering systematic administration of data and analyses. For example, an analyst can import the SDK into a Jupyter notebook to automatically access versioned algorithmic output for downstream post-processing, ensuring a full audit trail of data provenance from raw data to analysis and post-processing results. Moreover, CLI automatically creates and maintains easily accessible project directories with symbolic links pointing to all data and results, thus allowing access independently from the RESTful API.

Integration of analytical applications: in various embodiments, the system as a bioinformatics framework is completely agnostic to bioinformatics pipelines and does not include pre-built applications (e.g. variant callers such as Pindel, Strelka) or Workflow Management Systems (WMS; e.g. Bpipe, Toil). Nevertheless, end-users can package, install, and deploy applications of choice in accordance with their data and operational requirements. This enables full leverage of functionality while maintaining complete independence and flexibility in analytical workflows.

In various embodiments, to facilitate seamless integration and rapid iteration of data processing pipelines into the system, “Toil Container” and “Cookiecutter Toil” were developed. Cookiecutter Toil is a templating utility that creates tools or pipelines with built-in software development best practices (i.e. version control, containerization, cloud testing, packaging, documentation). On the other hand, Toil Container enables Toil class-based pipelines to perform containerized system calls with both Docker and Singularity without source code changes. Toil Container ensures that analytical logic remains independent of execution logic by keeping pipelines agnostic to containerization technology or compute environment (e.g. an application can run using Docker in the cloud or Singularity in LSF).

In various embodiments, there are two levels to system data access: interaction with metadata, and interaction with data. Metadata: Users can create, retrieve, update, and delete metadata using Web and API. In order to manage these interactions, the system relies on Django Permissions. By assigning users to groups, the administrator can manage the actions granted towards different resources. The system may offer 3 main roles: (1) Managers are users who can register samples, (2) analysts can run analyses, and (3) engineers can do both, register samples and run analyses. These roles are optional and customizable. Permissions can also be modified to each user specifically. Data: The system data lake can reside in the cloud or in a local file system. Access to these resources is not managed by the system but by a system administrator (i.e. Unix, Cloud). Users that have access to the data lake can execute applications if they have the right metadata permissions (e.g. create and update analyses). Once data is imported and analyses are finished, Isabl removes write permissions to prevent accidental deletion of data. Permissions to download and access data through Isabl Web are managed using Django Permissions.

Embodiments of the system address the major challenges in production-grade computational workflows. This includes the disruption of data silos, flexible integration to metadata sources, dynamic access and visualization of data, version control, audit trail, data harmonization, scalability, automation of analytical workflows and resource management (personnel as well as compute). Embodiments of the disclosed approach maintain a real time audit trail of each step in the data generation process. Results and related metadata are accessible and visualized through CLI and Web. Advantageously, growth in data footprint across time does not impose further demands on personnel. Embodiments of the system foster autonomy, automation, audit trail, and scalable deployment of data processing tools in a system-wide approach. Meta analyses of existing data sets represent a powerful means to derive new insights. Datasets may be combined to improve statistical power or new algorithms can be executed across projects for novel readouts. For example, the system facilitates the fast registration and processing of large number of patients from cohorts using a novel copy number analysis tool. Deployment of the tool may involve a two step process: (1) application registration; and (2) execution across samples that match a specific criteria (i.e. targeted sequencing technique equals IMPACT). In an embodiment, more than 35K analyses were submitted with a single command and processed in 3 days with a + 5K cpu HPC cluster. Resulting output files may be harmonized (same version) and organized under a specified project directory. Similarly, these principles apply to error correction in analytical workflows. Upon discovery of an error or “bug”, the system enables the identification of all affected experimental data, re-execution of analyses with a corrected application, and identification of all relevant stakeholders for notification of data status. The pre-existing analyses are transferred to a time-stamped legacy directory. During results retrieval end-users have automatic access to the latest version of each analyses run, but if desired, can retrieve older analyses files from the legacy directory. In one embodiment, the system implements an automated production-grade workflow for whole genome (WGS) and RNA analysis, executing more than 30 independent algorithms automatedly. CLI and institutional API integrations facilitate the registration of FASTQ files from a sequencing core. Upon import, automations can be used to deploy data processing applications (e.g. alignment, gene counts). Intermediate applications were subsequently executed as prior dependencies were satisfied (e.g. quality-control, variant calling). Also, derivation of summary statistics such as microsatellite instability and homologous DNA recombination scores that depend on primary data extraction (i.e. indels) were executed. Select data was embedded in a patient-centric report accessible through Web. Termed as the no-click genome, the entire process is executed with no manual intervention. These automations enable the discovery of novel diagnostic and therapy informing biomarkers within clinically relevant timeframes. The system supports the implementation of production-ready workflows. The no-click genome has completed reports at a rate of 4.5 ± 2 days / report (mean ± standard deviation; n = 20; mean depth coverage 80 ± 20) using a 3000-cores High Performance Computing multi-user cluster. Processing duration is primarily driven by the longest-running application at each parallel block as well as compute availability (i.e. cluster congestion)

In various embodiments, both platform and analysis paradigms make no assumptions about the nature of the data being registered. For a given individual, sequencing data as well as pathology data can be linked to specific samples. The same is true for analysis applications, for example a tiling preprocessing step could be productionized for new pathology images for a biopsy for which whole genome sequencing data is also produced. Analysis output files from image and whole genome sequencing variant calls are linked for a given individual. In this way, the system can facilitate the integration of diverse data modalities for downstream correlative analyses, which represents an area of increasing research focus.

In various embodiments, the system’s unconventional features include: (1) integration of a “RESTful API first” approach, (2) support for multimodal data, (3) an implementation agnostic to specific pipelines, workflow management systems, and storage and compute architectures, and (4) it’s “plug and play” deployability. The consolidation of these features into a platform is unique.

The development of digital biobanks can serve as companion infrastructures to support dynamic data access, processing and visualization of the growing data capital in research and healthcare. The system is able to support end-to-end bioinformatics operations. With the disclosed approach, real world challenges in computational biology, such as quality and version control, analysis audit trails, error correction, scalability, automation, and meta analyses can be readily addressed. To reduce the adoption barrier, the database schema can be customized and analysis tools can be added as Applications per end user specifications. To facilitate integration of analytical pipelines in accordance with best practices, Toil Container and Cookiecutter Toil have been developed as templating utilities that can be extended to include analyses pipelines for any data modality (NGS, single cell, imaging, etc.). Moreover, as a platform that facilitates and automates large scope institutional initiatives, RESTful API and CLI integrated with biospecimens databases, clinical resources, visualization platforms, sequencing cores, and laboratory information management systems. Implementation of computable digital biobanks helps minimize costs by efficiently managing compute resources, reducing time to analyses and importantly demands for hands on operator time to process data. These automations at the same time maximize data deliverables, utilization of the data capital and reproducibility of findings.

In various embodiments, the system architecture is built upon separate codebases, which are loosely coupled and can be deployed independently in a plug-and-play fashion. For example, Web services only dependency is Docker Compose, while the command line client is distributed using the Python Package Index. Furthermore, the system’s metadata infrastructure is decoupled and agnostic of compute and data storage environments (e.g. local, cluster, cloud). This functionality separates dependencies, fosters interoperability across data processing environments, and ensures that metadata is accessible even when the data is no longer available (FAIR A2). Further, API and CLI may be installed as external dependencies, guaranteeing compatibility with future upgrades. As a result, end-users don’t have to alter source code to extend or modify the platform functionality (i.e. adding support for diverse data modalities such as imaging, radiology etc.). The user interface may be an interactive single-page application developed delivered as a node package through the package manager NPM

Abbreviations. AIMS: Analysis information management system; WGS: Whole genome sequencing; NGS: Next generation sequencing; CLI: Command line interface; SPA: Single page application; HPC: High performance computing; UUID: Universally unique identifier; WMS: Workflow management system.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A computer-implemented method comprising:
(A) generating, based on sequencing of a tumor sample and a healthy control germline sample, a plurality of datasets comprising: (1) a first dataset based on whole transcriptome sequencing of RNA in the tumor sample obtained from a patient; (2) a second dataset based on a whole genome sequencing (WGS) of DNA derived from the tumor sample obtained from the patient; and (3) a third dataset based on WGS of DNA in the healthy control germline sample;
(B) accessing a plurality of databases comprising: (1) a first reference database comprising, for a reference cohort of tumor samples, a plurality of individual sample gene expression transcripts per million (TPM) values; (2) a second reference database comprising, for a reference cohort of tumor samples, at an individual sample level, annotations for at least one of (i) RNA fusions, (ii) somatic structural variants, (iii) somatic substitutions, (iv) somatic insertions and deletions (indels), (v) microsatellite instability and/or mutational burden scores for each variant class, (vi) germline variants, (vii) somatic mutation patterns or signatures in each sample, or (vii) allelic imbalances; and (3) a third database comprising a plurality of gene identifiers corresponding to a plurality of known cancer genes;
(C) performing an RNA gene expression analysis using the first dataset, the first reference database, and the third database, to generate, for the tumor sample, a first plurality of outputs based on: (1) detection of established cancer genes having aberrant gene expression in the tumor sample relative to that observed in normal control subjects; and (2) prioritization of the detected aberrantly-expressed cancer genes in the tumor sample;
(D) performing a DNA ploidy and allelic imbalance analysis using the second dataset, the third dataset, and the third database, to generate, for the tumor sample, a second plurality of outputs based on: (1) detection of high-confidence aberrant copy number segments in the tumor sample by applying one or more allelic imbalance identification techniques; and (2) prioritization of allelic imbalances in the tumor sample based on a set of criteria comprising an overlap of the high-confidence aberrant copy number segments in the tumor sample with the known cancer genes in the third database;
(E) performing, based on the RNA gene expression analysis of Step (C) and the DNA ploidy and allelic imbalance analysis of Step (D), a variant calling analysis to generate, for the tumor sample, a third plurality of outputs based on: (1) detection of RNA fusions; (2) detection of somatic structural variants; (3) detection of somatic substitutions; (4) detection of somatic insertions and deletions (indels); (5) assessment of microsatellite instability and/or mutational burden across variant classes; (6) detection of germline variants; (7) clonality analysis; (8) determination of a number of structural variants and gene fusions in the DNA of the tumor sample; and/or (9) determination of somatic mutation patterns or signatures in the tumor sample; and
(F) implementing a workflow comprising: (1) identifying orthogonal supportive indicators based on consistency of two or more outputs in at least two of the first, second, and third pluralities of outputs generated in Step (C), Step (D), and Step (E), respectively; (2) prioritizing genetic alterations based on the orthogonal supportive indicators; (3) generating global classifications based on the orthogonal supportive indicators; and (4) classifying at least one somatic mutation in an established cancer gene that is detected in both the first dataset and the second dataset as being orthogonally validated; (G) generating cohort classification scores for each individual level output in each of the first, second, and third pluralities of outputs for the tumor sample based on the reference cohort of tumor samples in at least one of the first reference database or the second reference database;
(H) generating disease-specific classification scores for each individual level output in each of the first, second, and third pluralities of outputs for the tumor sample based on a subset of the reference cohort of tumor samples in at least one of the first reference database or the second reference database, wherein the subset of the reference cohort of tumor samples is of a same cancer type;
(I) generating a report comprising, for the tumor sample, the prioritized allelic imbalances, the microsatellite instability and/or mutational burden across variant classes, the germline variants, outputs of the clonality analysis, the number of structural variants and gene fusions in the DNA, the somatic mutation patterns or signatures, the at least one orthogonally-validated somatic mutation, the cohort classification scores, and the disease specific classification scores; and
(J) providing the report to one or more users for determination of an anti-cancer therapy, wherein providing the report comprises at least one of (1) transmitting the report to a computing device, (2) displaying the report on a display screen, or (3) storing the report in a non-volatile computer-readable storage medium that is accessible to the one or more users.

2. The computer-implemented method of claim 1, wherein generating the cohort classification scores further comprises: (1) interrogating the at least one orthogonally-validated somatic mutation against a fourth database that associates a plurality of somatic mutations with a plurality of specific cancer types or pan-cancer markers; and (2) identifying the at least one orthogonally-validated somatic mutation as associated with a specific cancer type or pan-cancer hotspot when there is a match.

3. The computer-implemented method of claim 1, further comprising classifying germline mutation pathogenicity by integration of data derived in Step 1(E) relating to acquired somatic mutation patterns or signatures.

4. The computer-implemented method of claim 1, further comprising determining the anti-cancer therapy based on values used for the report, and providing the anti-cancer therapy in the report.

5. The computer-implemented method of claim 1, further comprising determining that the second and third datasets have at least one of (1) a quality score satisfying a quality threshold, wherein the quality score indicates genome mapping quality, and the quality threshold is at least 20 Phred, (2) a coverage metric satisfying a coverage threshold, wherein the coverage score indicates genome coverage, and the coverage threshold is at least about 70% genome coverage, or (3) a tumor cell content satisfying a tumor purity threshold, wherein the tumor cell content indicates tumor purity corresponding to the DNA in the tumor sample, and the tumor purity threshold is at least about 20% tumor purity.

6. The computer-implemented method of claim 1, wherein performing the RNA gene expression analysis further comprises detecting over-expressed or under-expressed genes based on the TPM values satisfying a percentile threshold relative to the first reference database.

7. The computer-implemented method of claim 1, wherein the set of criteria for prioritizing allelic imbalances in the tumor sample further includes at least one of whole-genome duplication (WGD) or an aberrant copy number segment having a direction that is consistent with cancer gene function.

8. The computer-implemented method of claim 1, the variant calling analysis comprising at least one of: (i) detection of RNA fusions, wherein detection of RNA fusions comprises employing a plurality of independent fusion gene callers on raw data, (ii) detection of RNA fusions, wherein detection of RNA fusions comprises detection of high-confidence fusion genes, and employing a rescue process to recover detected high-confidence fusion genes that were not detected by at least two independent variant callers as a reference for known cancer genes, wherein rescued fusions are required to have at least one spanning read, (iii) detection of somatic structural variants, wherein detection of somatic structural variants comprises deploying a plurality of independent structural variant callers on raw data, (iv) detection of somatic structural variants, wherein detection of somatic structural variants comprises selection of high-confidence structural variants by merging all calls having more than a first predetermined number of base pairs (bp) by a window that includes a breakpoint, the window having a size that is a second predetermined number of bps, (v) detection of somatic substitutions, wherein detection of somatic substitutions comprises employing a plurality of independent substitution callers on raw data, (vi) detection of somatic indels, wherein detection of somatic indels comprises generating one or more indel signatures and using the one or more indel signatures to determine if a somatic indel is a repeat-mediated deletion, a microhomology association, or an insertion, (vii) assessment of microsatellite instability and/or mutational burden across variant classes, or (viii) detection of germline variants, wherein detection of germline variants comprises deploying a plurality of independent germline callers on raw data.

9. The computer-implemented method of claim 1, further comprising determining structural variant (SV) burden by collapsing complex structural variants into unique structural variant clusters to avoid over estimation of structural variant burden.

10. The computer-implemented method of claim 1, wherein the clonality analysis comprises using purity and local copy numbers to scale variant allele frequency (VAF) of single nucleotide variants (SNVs) and indels to cancer cell fraction (CCF) for one or more tumor samples from the patient, wherein (1) candidate driver mutations, (2) aberrant copy number segments, and (3) structural variants are assigned to each clone to generate clone-specific mutation profiles.

11. A computer-implemented method comprising:
(A) generating, by one or more processors of a computing system, based on sequencing of a tumor sample and a healthy control germline sample, a plurality of datasets comprising: (1) a first dataset based on whole transcriptome sequencing of RNA in the tumor sample obtained from a patient; (2) a second dataset based on a whole genome sequencing (WGS) of DNA derived from the tumor sample obtained from the patient; and (3) a third dataset based on WGS of DNA in the healthy control germline sample;
(B) performing, by the one or more processors, an RNA gene expression analysis using the first dataset to generate, for the tumor sample, a first plurality of outputs based on detection of established cancer genes having aberrant gene expression in the tumor sample relative to that observed in normal control subjects;
(C) performing, by the one or more processors, a DNA ploidy and allelic imbalance analysis using the second dataset to generate, for the tumor sample, a second plurality of outputs based on detection of high-confidence aberrant copy number segments in the tumor sample by applying one or more allelic imbalance identification techniques;
(D) performing, by the one or more processors, based on the RNA gene expression analysis of Step (B) and the DNA ploidy and allelic imbalance analysis of Step (C), a variant calling analysis to generate, for the tumor sample, a third plurality of outputs based on a plurality of: (1) detection of RNA fusions; (2) detection of somatic structural variants; (3) detection of somatic substitutions; (4) detection of somatic insertions and deletions (indels); (5) assessment of microsatellite instability and/or mutational burden across variant classes; (6) detection of germline variants; (7) clonality analysis; (8) determination of a number of structural variants and gene fusions in the DNA of the tumor sample; and/or (9) determination of somatic mutation patterns or signatures in the tumor sample; and
(E) implementing, by the one or more processors, a workflow comprising: (1) identifying, by the one or more processors, orthogonal supportive indicators based on consistency of two or more outputs in at least two of the first, second, and third pluralities of outputs generated in Steps (B), (C), and (D), respectively; (2) classifying, by the one or more processors, at least one somatic mutation in an established cancer gene that is detected in both the first dataset and the second dataset as being orthogonally validated;
(F) generating, by the one or more processors, cohort classification scores for each individual level output in each of the first, second, and third pluralities of outputs for the tumor sample based on a reference cohort of tumor samples;
(G) generating, by the one or more processors, disease-specific classification scores for each individual level output in each of the first, second, and third pluralities of outputs for the tumor sample based on a subset of the reference cohort of tumor samples, wherein the subset of the reference cohort of tumor samples is of a same cancer type;
(H) generating, by the one or more processors, a report comprising, for the tumor sample, based on Steps (A)-(G), information corresponding to a plurality of allelic imbalances, microsatellite instability and/or mutational burden across variant classes, germline variants, clonality analysis, structural variants and gene fusions in the DNA, somatic mutation patterns or signatures, orthogonally-validated somatic mutations, cohort classification scores, and disease specific classification scores;
(I) providing, by the one or more processors, the report to one or more users for determination of an anti-cancer therapy, wherein providing the report comprises at least one of (1) transmitting, by the one or more processors, the report to a computing device, (2) displaying, by the one or more processors, the report on a display screen, or (3) storing the report in a non-volatile computer-readable storage medium of the computing system.

12. The computer-implemented method of claim 11, further comprising determining the anti-cancer therapy based on values used for the report, and providing the anti-cancer therapy in the report, wherein the anti-cancer therapy is determined based on interrogation of a therapy database to identify a therapy that aligns with the outputs in the report.

13. A computer-implemented method comprising:
(A) performing, by one or more processors of a computing system, based on a first plurality of outputs from an RNA gene expression analysis and a second plurality of outputs from a DNA ploidy and allelic imbalance analysis, a variant calling analysis to generate, for a tumor sample, a third plurality of outputs based at least on two or more of: (1) detection of RNA fusions; (2) detection of somatic structural variants; (3) detection of somatic substitutions; (4) detection of somatic insertions and deletions (indels); (5) assessment of microsatellite instability and/or mutational burden across variant classes; (6) detection of germline variants; (7) clonality analysis; (8) determination of a number of structural variants and gene fusions in the DNA of the tumor sample; or (9) determination of somatic mutation patterns or signatures in the tumor sample;
(B) generating, by the one or more processors, for the tumor sample, a report comprising two or more of: (1) prioritized allelic imbalances; (2) microsatellite instability or mutational burden across variant classes; (3) the detected germline variants; (4) outputs of the clonality analysis; (5) the number of structural variants and gene fusions in the DNA; or (6) the somatic mutation patterns or signatures; and
(C) providing, by the one or more processors, the report for determination of an anti-cancer therapy.

14. The computer-implemented method of claim 13:
(1) wherein the first plurality of outputs from the RNA gene expression analysis is based on detection of established cancer genes having aberrant gene expression in the tumor sample relative to that observed in normal control subjects, and on prioritization of the detected aberrantly-expressed cancer genes in the tumor sample, and
(2) wherein the second plurality of outputs from the DNA ploidy and allelic imbalance analysis is based on detection of high-confidence aberrant copy number segments in the tumor sample by applying one or more allelic imbalance identification techniques, and on prioritization of allelic imbalances in the tumor sample based on a set of criteria comprising an overlap of the high-confidence aberrant copy number segments in the tumor sample with known cancer genes.

15. The computer-implemented method of claim 13, wherein:
the RNA gene expression analysis is performed using a first dataset corresponding to whole transcriptome sequencing of RNA in the tumor sample obtained from a patient; and
the DNA ploidy and allelic imbalance analysis is performed using a second dataset corresponding to a whole genome sequencing (WGS) of DNA derived from the tumor sample obtained from the patient, and a third dataset corresponding to WGS of DNA in the healthy control germline sample.

16. The computer-implemented method of claim 13, further comprising:
(A) identifying, by the one or more processors, orthogonal supportive indicators based on consistency of two or more outputs in at least two of the first plurality of outputs, the second plurality of outputs, and the third plurality of outputs;
(B) prioritizing, by the one or more processors, genetic alterations based on the orthogonal supportive indicators;
(C) generating, by the one or more processors, global classifications based on the orthogonal supportive indicators; and
(D) classifying, by the one or more processors, at least one somatic mutation in an established cancer gene that is detected in both the first dataset and the second dataset as being orthogonally validated.

17. The computer-implemented method of claim 13, further comprising generating, by the one or more processors, cohort classification scores for each individual level output in each of the first plurality of outputs, the second plurality of outputs, and the third plurality of outputs for the tumor sample based on a reference cohort of tumor samples in at least one of a first reference database or a second reference database,
(1) wherein the first reference database comprises, for the reference cohort of tumor samples, a plurality of individual sample gene expression transcripts per million (TPM) values,
(2) wherein the second reference database comprises, for the reference cohort of tumor samples, at an individual sample level, annotations for at least one of (i) RNA fusions, (ii) somatic structural variants, (iii) somatic substitutions, (iv) somatic insertions and deletions (indels), (v) microsatellite instability or mutational burden scores for each variant class, (vi) germline variants, (vii) somatic mutation patterns or signatures in each sample, or (vii) allelic imbalances, and
(3) wherein the report further comprises the cohort classification scores.

18. The computer-implemented method of claim 17, wherein generating the cohort classification scores comprises:
(1) interrogating the at least one orthogonally-validated somatic mutation against a fourth database that associates a plurality of somatic mutations with a plurality of specific cancer types or pan-cancer markers; and
(2) identifying the at least one orthogonally-validated somatic mutation as associated with a specific cancer type or pan-cancer hotspot when there is a match.

19. The computer-implemented method of claim 13, further comprising generating, by the one or more processors, disease-specific classification scores for each individual level output in each of the first plurality of outputs, the second plurality of outputs, and the third plurality of outputs for the tumor sample based on a subset of a reference cohort of tumor samples in at least one of a first reference database or a second reference database,
(1) wherein the first reference database comprises, for the reference cohort of tumor samples, a plurality of individual sample gene expression transcripts per million (TPM) values,
(2) wherein the second reference database comprises, for the reference cohort of tumor samples, at an individual sample level, annotations for at least one of (i) RNA fusions, (ii) somatic structural variants, (iii) somatic substitutions, (iv) somatic insertions and deletions (indels), (v) microsatellite instability and/or mutational burden scores for each variant class, (vi) germline variants, (vii) somatic mutation patterns or signatures in each sample, or (vii) allelic imbalances, and
(3) wherein the report further comprises the disease-specific classification scores.

20. The computer-implemented method of claim 13, further comprising determining the anti-cancer therapy based on values used for the report, and providing the anti-cancer therapy in the report.
